SEQUENCE LISTING (28 sequences)

SEQ ID NO: 1 - DNA, 3776 bp, Homo sapiens
cattcataagactcagagctacggccacggcagggacacgcggaaccaagacttggaaacttgattgttgtggttcttcttgggggttatgaaatttcattaatctttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgccgcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacgctcaccccagaggaagaggagaagcgaagggtgcgccgggaacgaaataaactagcagcagctaaatgcaggaaccggcggagggagctgaccgaccgactccaggcggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctctgtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggattaagggacgggggtgggaggtaggctgtggggtgggctggagtcctctccagagaggctcaacaaggaaaaatgccactccctacccaatgtctcccacacccaccctttttttggggtgcctaggttggtttcccctgcactcccgaccttagcttattgatcccacatttccatggtgtgagatcctctttactctgggcagaagtgagccccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaagggagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgca

SEQ ID NO: 2 - protein, 338 aa, Homo sapiens
MFQAFPGDYDSGSRCSSSPSAESQYLSSVDSFGSPPTAAASQECAGLGEMPGSFVPTVTAITTSQDLQWLVQPTLISSMAQSQGQPLASQPPVVDPYDMPGTSYSTPGMSGYSSGGASGSGGPSTSGTTSGPGPARPARARPRRPREETLTPEEEEKRRVRRERNKLAAAKCRNRRRELTDRLQAETDQLEEEKAELESEIAELQKEKERLEFVLVAHKPGCKIPYEEGPGPGPLAEVRDLPGSAPAKEDGFSWLLPPPPPPPLPFQTSQDAPPNLTASLFTHSEVQVLGDPFPVVNPSYTSSFVLTCPEVSAFAGAQRTSGSDQPSDPLNSPSLLAL

SEQ ID NO: 3 - DNA, 3668 bp, Homo sapiens
cattcataagactcagagctacggccacggcagggacacgcggaaccaagacttggaaacttgattgttgtggttcttcttgggggttatgaaatttcattaatctttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgccgcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctctgtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggattaagggacgggggtgggaggtaggctgtggggtgggctggagtcctctccagagaggctcaacaaggaaaaatgccactccctacccaatgtctcccacacccaccctttttttggggtgcctaggttggtttcccctgcactcccgaccttagcttattgatcccacatttccatggtgtgagatcctctttactctgggcagaagtgagccccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaagggagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgca

SEQ ID NO: 4 - protein, 302 aa, Homo sapiens
MFQAFPGDYDSGSRCSSSPSAESQYLSSVDSFGSPPTAAASQECAGLGEMPGSFVPTVTAITTSQDLQWLVQPTLISSMAQSQGQPLASQPPVVDPYDMPGTSYSTPGMSGYSSGGASGSGGPSTSGTTSGPGPARPARARPRRPREETETDQLEEEKAELESEIAELQKEKERLEFVLVAHKPGCKIPYEEGPGPGPLAEVRDLPGSAPAKEDGFSWLLPPPPPPPLPFQTSQDAPPNLTASLFTHSEVQVLGDPFPVVNPSYTSSFVLTCPEVSAFAGAQRTSGSDQPSDPLNSPSLLAL

SEQ ID NO: 5 - DNA, 4679 bp, Homo sapiens
gagccaggcgggggcggagggggaggctggcagcggagctttgaatagggaagttttgcaggggttacgtttgcagtcagtccggtgtttgcaaatattgtgtgggctccgcgcgctgcgggctgcgggagggtccggccgggcgtctctgcgagcctggagtttgcatgaaactttcacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatggtatgtgggtgaccctggactcgccactggtacccgcccttccgagcggcgcctaagcctttgccgtgagcatgcacactgagaatgctaatggttgggttgattgtatgttgaggatctattactgaccgtatgatgaggccaactttttttccttgtggttagcaagactgcaagagatggaaaaaaagtagtttgaatgttttgtgtgtaaggagtataccatgagatgagatgaccaccaatcatttccttggggggagggggtgtctgcaccttagaaaaaaaaagaaaaatcaaaaaaacaaaaaaacaaaaacaaaaaaagaaggaaaatcttggagggtgggcgtgggaactcaggaccccagagtggcgagtggtgtggggagggagagcctctctcccccttttctgtgtgagaggaactcttagtgtctggtgcagctattaaatgtgcaatgtgtcaagtagcttgttttacacgctacaacatagctcatttgtaacccattgtataagctgtgtatttacaaatataacacaacaatttaacttttccttagaatacaaaaagtcatgcatggtctggggaactatatgcttttccatttttaagtcaggactgcaatactgattccagttaatgagcagctaagatccaatctgtctaatacagtgaccccctagccatccgggcctggcaatatacaattttttttcccctccaagtttgtaacactccccttccagaaaggcattgtgcaacacaggattatttttaaatgattctgaatttgaattaactttttggagaattctgtgatgcccttagaagaaattggacacgtattgagtgtcacaaagctggggctgggaattgctggtctaatgtttcattagacttaagaacctaaaatttttctcagttgggtggataaaaccactaacgcttagaaactgttttctcatgcagctatgtttctcttatttatgccttgaggactaatttctggttttctagctgttaatgcactgttgaccttcataatggtgccttacgcaagcgatcccttctgtgggggtctcatacaggggtgtgggcgatgcatgctttattaaggctcttgtttcacctggcagtgtactgtatcaacgtataatacagaaaaaaaatctctttaaggtcctccttcacaaagacatagagtgaaactccctttacatgtcagtatttgttcaacactttaggcaacttgactgtcagtgttaaaatggaaaacaggaaaatggaaaaatctgaccaattctgccaccttgagactttcatatagaccttgcacaacaattgtatagatcacacaccggctgtatttaatatgtaacattttcacacatattaaagatacagaagtattaaaaaacccccaatgttaatgtatttgcttaaaaggcacaagtttcacatatctgtctagctatctgttggtaatacagaaagtatactacttttttaaaaaagtgggcagaattcttgtgtatgtatatttgtgtgtacagtatgtgtatgtgtgtatatatatatattatatatatagataatatataaatattttttttaaggagaaactagaatgtttagctagaaaattccacagcctgtgaagaaatatttcaaaatggccataaaggaggtaaaaatgaaaaccataacctaacttttatagaggctttatctttaatttaacgatgtgcggaggactttcttgcttgaatctgttccgggctgtctgctctgtccatcaaatgggcaggtctggaatggggcaccttcggccgttcagaagtggcctgaacagaatgctggaacccaggctggactcggacacactaaggttttgattttgaatttcagccttattagaagatctaacctaagagtaagctaaccacagggattcttttgtagaacactttttatgcagatgaagctattttttccagcaagtagattcttccagtttttccaaggagtaatttccccgaattggcataccacggcgtggacagctgatatttcacccagctgctggcttgtgggtgtggctctttgctttatatatatatacacacatgtgagtctggctgggctggtattttgtttgatcttcctggaaatgagcagtgactaacgctcacataactggttttttttttatctgggctgatgaatacatttacctaagaaactcatttcgttttacttaagaggggaagtgcagttttcttttggcagttcagaatccaagcacttgatttgctgggtttggaaaactccttttttggccttctatgtgcttagccataacaattccattaagcaagaaggtaagcaaaagacaaaaaaaaaaaaaggaaaaaaaaaaacttgcactggcttgtctcacttacgaaacatgtcggagctgtttgcctgggtggggctgggtaccgtacctgtcaatgcctgtgattttcataattagcacgtacataaagaagtacattctgttcaggtgataactgagcctcaatcaagcagaaactttttgcttgaaattaaaaaaaaatttctattagtgaaatttcttttttttttttttttggaagcaccctgttatctaaagaatctttgtaagatttttgtaaaattttgttttacaagattttatttgaaattgttttttgcaagattgttatatttctgtatgaatgtattttttattggaataacataaaagaattcttatcagcatcttgagtctggttgttttttttgggggaagggggttgttggaacagattcctcctgcattcatcaccctggcttcctcctgggggcaaatcttcattgagcaaccctgagaacaactcaccgacctcagccccttcctctttccacagcctgtcctgggagctggagaggatgtcagcgagcctgacattgccctccctgaatgcatcaaatactcttctccaaggactgacaaaaacaaccccccggctgtgggcacagtagggaaccatgccggtttgatcttccattgctcaagccagggaatgcattgcagaggcacctgcacaacgacaaacttgagtgtttccctgccacttctgtgcctccacagcctgctccagttccgcagctagagctgggcctaccttccccagccagccctgccacacacctggctgaggcatgtctgggaggggtggaggaggcagagggactccaggccagagggctgttctcacaacctcagcccactctcagtcaaggagggcagaaaataaaagatgacatcactgccaaaaaaaaaaaaaaaaaa

SEQ ID NO: 6 - protein, 283 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGEKPYRCSWEGCEWRFARSDELTRHFRKHTGAKPFKCSHCDRCFSRSDHLALHMKRHL

SEQ ID NO: 7 - DNA, 4553 bp, Homo sapiens
gagccaggcgggggcggagggggaggctggcagcggagctttgaatagggaagttttgcaggggttacgtttgcagtcagtccggtgtttgcaaatattgtgtgggctccgcgcgctgcgggctgcgggagggtccggccgggcgtctctgcgagcctggagtttgcatgaaactttcacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatggtatgtgggtgaccctggactcgccactggtacccgcccttccgagcggcgcctaagcctttgccgtgagcatgcacactgagaatgctaatggttgggttgattgtatgttgaggatctattactgaccgtatgatgaggccaactttttttccttgtggttagcaagactgcaagagatggaaaaaaagtagtttgaatgttttgtgtgtaaggagtataccatgagatgagatgaccaccaatcatttccttggggggagggggtgtctgcaccttagaaaaaaaaagaaaaatcaaaaaaacaaaaaaacaaaaacaaaaaaagaaggaaaatcttggagggtgggcgtgggaactcaggaccccagagtggcgagtggtgtggggagggagagcctctctcccccttttctgtgtgagaggaactcttagtgtctggtgcagctattaaatgtgcaatgtgtcaagtagcttgttttacacgctacaacatagctcatttgtaacccattgtataagctgtgtatttacaaatataacacaacaatttaacttttccttagaatacaaaaagtcatgcatggtctggggaactatatgcttttccatttttaagtcaggactgcaatactgattccagttaatgagcagctaagatccaatctgtctaatacagtgaccccctagccatccgggcctggcaatatacaattttttttcccctccaagtttgtaacactccccttccagaaaggcattgtgcaacacaggattatttttaaatgattctgaatttgaattaactttttggagaattctgtgatgcccttagaagaaattggacacgtattgagtgtcacaaagctggggctgggaattgctggtctaatgtttcattagacttaagaacctaaaatttttctcagttgggtggataaaaccactaacgcttagaaactgttttctcatgcagctatgtttctcttatttatgccttgaggactaatttctggttttctagctgttaatgcactgttgaccttcataatggtgccttacgcaagcgatcccttctgtgggggtctcatacaggggtgtgggcgatgcatgctttattaaggctcttgtttcacctggcagtgtactgtatcaacgtataatacagaaaaaaaatctctttaaggtcctccttcacaaagacatagagtgaaactccctttacatgtcagtatttgttcaacactttaggcaacttgactgtcagtgttaaaatggaaaacaggaaaatggaaaaatctgaccaattctgccaccttgagactttcatatagaccttgcacaacaattgtatagatcacacaccggctgtatttaatatgtaacattttcacacatattaaagatacagaagtattaaaaaacccccaatgttaatgtatttgcttaaaaggcacaagtttcacatatctgtctagctatctgttggtaatacagaaagtatactacttttttaaaaaagtgggcagaattcttgtgtatgtatatttgtgtgtacagtatgtgtatgtgtgtatatatatatattatatatatagataatatataaatattttttttaaggagaaactagaatgtttagctagaaaattccacagcctgtgaagaaatatttcaaaatggccataaaggaggtaaaaatgaaaaccataacctaacttttatagaggctttatctttaatttaacgatgtgcggaggactttcttgcttgaatctgttccgggctgtctgctctgtccatcaaatgggcaggtctggaatggggcaccttcggccgttcagaagtggcctgaacagaatgctggaacccaggctggactcggacacactaaggttttgattttgaatttcagccttattagaagatctaacctaagagtaagctaaccacagggattcttttgtagaacactttttatgcagatgaagctattttttccagcaagtagattcttccagtttttccaaggagtaatttccccgaattggcataccacggcgtggacagctgatatttcacccagctgctggcttgtgggtgtggctctttgctttatatatatatacacacatgtgagtctggctgggctggtattttgtttgatcttcctggaaatgagcagtgactaacgctcacataactggttttttttttatctgggctgatgaatacatttacctaagaaactcatttcgttttacttaagaggggaagtgcagttttcttttggcagttcagaatccaagcacttgatttgctgggtttggaaaactccttttttggccttctatgtgcttagccataacaattccattaagcaagaaggtaagcaaaagacaaaaaaaaaaaaaggaaaaaaaaaaacttgcactggcttgtctcacttacgaaacatgtcggagctgtttgcctgggtggggctgggtaccgtacctgtcaatgcctgtgattttcataattagcacgtacataaagaagtacattctgttcaggtgataactgagcctcaatcaagcagaaactttttgcttgaaattaaaaaaaaatttctattagtgaaatttcttttttttttttttttggaagcaccctgttatctaaagaatctttgtaagatttttgtaaaattttgttttacaagattttatttgaaattgttttttgcaagattgttatatttctgtatgaatgtattttttattggaataacataaaagaattcttatcagcatcttgagtctggttgttttttttgggggaagggggttgttggaacagattcctcctgcattcatcaccctggcttcctcctgggggcaaatcttcattgagcaaccctgagaacaactcaccgacctcagccccttcctctttccacagcctgtcctgggagctggagaggatgtcagcgagcctgacattgccctccctgaatgcatcaaatactcttctccaaggactgacaaaaacaaccccccggctgtgggcacagtagggaaccatgccggtttgatcttccattgctcaagccagggaatgcattgcagaggcacctgcacaacgacaaacttgagtgtttccctgccacttctgtgcctccacagcctgctccagttccgcagctagagctgggcctaccttccccagccagccctgccacacacctggctgaggcatgtctgggaggggtggaggaggcagagggactccaggccagagggctgttctcacaacctcagcccactctcagtcaaggagggcagaaaataaaagatgacatcactgccaaaaaaaaaaaaaaaaaa

SEQ ID NO: 8 - protein, 241 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGEKPYRCSWEGCEWRFARSDELTRHFRKHTGAKPFKCSHCDRCFSRSDHLALHMKRHL

SEQ ID NO: 9 - DNA, 4555 bp, Homo sapiens
gagccaggcgggggcggagggggaggctggcagcggagctttgaatagggaagttttgcaggggttacgtttgcagtcagtccggtgtttgcaaatattgtgtgggctccgcgcgctgcgggctgcgggagggtccggccgggcgtctctgcgagcctggagtttgcatgaaactttcacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatggtatgtgggtgaccctggactcgccactggtacccgcccttccgagcggcgcctaagcctttgccgtgagcatgcacactgagaatgctaatggttgggttgattgtatgttgaggatctattactgaccgtatgatgaggccaactttttttccttgtggttagcaagactgcaagagatggaaaaaaagtagtttgaatgttttgtgtgtaaggagtataccatgagatgagatgaccaccaatcatttccttggggggagggggtgtctgcaccttagaaaaaaaaagaaaaatcaaaaaaacaaaaaaacaaaaacaaaaaaagaaggaaaatcttggagggtgggcgtgggaactcaggaccccagagtggcgagtggtgtggggagggagagcctctctcccccttttctgtgtgagaggaactcttagtgtctggtgcagctattaaatgtgcaatgtgtcaagtagcttgttttacacgctacaacatagctcatttgtaacccattgtataagctgtgtatttacaaatataacacaacaatttaacttttccttagaatacaaaaagtcatgcatggtctggggaactatatgcttttccatttttaagtcaggactgcaatactgattccagttaatgagcagctaagatccaatctgtctaatacagtgaccccctagccatccgggcctggcaatatacaattttttttcccctccaagtttgtaacactccccttccagaaaggcattgtgcaacacaggattatttttaaatgattctgaatttgaattaactttttggagaattctgtgatgcccttagaagaaattggacacgtattgagtgtcacaaagctggggctgggaattgctggtctaatgtttcattagacttaagaacctaaaatttttctcagttgggtggataaaaccactaacgcttagaaactgttttctcatgcagctatgtttctcttatttatgccttgaggactaatttctggttttctagctgttaatgcactgttgaccttcataatggtgccttacgcaagcgatcccttctgtgggggtctcatacaggggtgtgggcgatgcatgctttattaaggctcttgtttcacctggcagtgtactgtatcaacgtataatacagaaaaaaaatctctttaaggtcctccttcacaaagacatagagtgaaactccctttacatgtcagtatttgttcaacactttaggcaacttgactgtcagtgttaaaatggaaaacaggaaaatggaaaaatctgaccaattctgccaccttgagactttcatatagaccttgcacaacaattgtatagatcacacaccggctgtatttaatatgtaacattttcacacatattaaagatacagaagtattaaaaaacccccaatgttaatgtatttgcttaaaaggcacaagtttcacatatctgtctagctatctgttggtaatacagaaagtatactacttttttaaaaaagtgggcagaattcttgtgtatgtatatttgtgtgtacagtatgtgtatgtgtgtatatatatatattatatatatagataatatataaatattttttttaaggagaaactagaatgtttagctagaaaattccacagcctgtgaagaaatatttcaaaatggccataaaggaggtaaaaatgaaaaccataacctaacttttatagaggctttatctttaatttaacgatgtgcggaggactttcttgcttgaatctgttccgggctgtctgctctgtccatcaaatgggcaggtctggaatggggcaccttcggccgttcagaagtggcctgaacagaatgctggaacccaggctggactcggacacactaaggttttgattttgaatttcagccttattagaagatctaacctaagagtaagctaaccacagggattcttttgtagaacactttttatgcagatgaagctattttttccagcaagtagattcttccagtttttccaaggagtaatttccccgaattggcataccacggcgtggacagctgatatttcacccagctgctggcttgtgggtgtggctctttgctttatatatatatacacacatgtgagtctggctgggctggtattttgtttgatcttcctggaaatgagcagtgactaacgctcacataactggttttttttttatctgggctgatgaatacatttacctaagaaactcatttcgttttacttaagaggggaagtgcagttttcttttggcagttcagaatccaagcacttgatttgctgggtttggaaaactccttttttggccttctatgtgcttagccataacaattccattaagcaagaaggtaagcaaaagacaaaaaaaaaaaaaggaaaaaaaaaaacttgcactggcttgtctcacttacgaaacatgtcggagctgtttgcctgggtggggctgggtaccgtacctgtcaatgcctgtgattttcataattagcacgtacataaagaagtacattctgttcaggtgataactgagcctcaatcaagcagaaactttttgcttgaaattaaaaaaaaatttctattagtgaaatttcttttttttttttttttggaagcaccctgttatctaaagaatctttgtaagatttttgtaaaattttgttttacaagattttatttgaaattgttttttgcaagattgttatatttctgtatgaatgtattttttattggaataacataaaagaattcttatcagcatcttgagtctggttgttttttttgggggaagggggttgttggaacagattcctcctgcattcatcaccctggcttcctcctgggggcaaatcttcattgagcaaccctgagaacaactcaccgacctcagccccttcctctttccacagcctgtcctgggagctggagaggatgtcagcgagcctgacattgccctccctgaatgcatcaaatactcttctccaaggactgacaaaaacaaccccccggctgtgggcacagtagggaaccatgccggtttgatcttccattgctcaagccagggaatgcattgcagaggcacctgcacaacgacaaacttgagtgtttccctgccacttctgtgcctccacagcctgctccagttccgcagctagagctgggcctaccttccccagccagccctgccacacacctggctgaggcatgtctgggaggggtggaggaggcagagggactccaggccagagggctgttctcacaacctcagcccactctcagtcaaggagggcagaaaataaaagatgacatcactgccaaaaaaaaaaaaaaaaaa

SEQ ID NO: 10 - protein, 237 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGVFPGLTTWPCT

SEQ ID NO: 11 - DNA, 3938 bp, Homo sapiens
gtactggcccgcgccgtccgcccgccgacagctccctgagccagcccgggaggcagccgcgcgcagcgagccggtggcgcaggtgtcggggtcctcgagcgcccagcctgggagcatgattgtggacaagctgctggacgacagccgcggcggagaggggctgcgggacgcggcgggcggctgcggcctcatgaccagcccgctcaacctgagctacttctacggcgcgtcgccgcccgccgccgccccgggcgcctgcgacgccagctgctcggtcttgggcccctcggcgcccggctcgcccggctccgactcctccgacttctcctctgcctcgtcggtgtcctcctgcggcgccgtggagtcccggtcgagaggcggcgcccgcgccgagcgccagccagttgagccccatatgggggttggcaggcagcagagaggcccctttcaaggtgttcgggtaaagaactcagtgaaggaactcctgttgcacatccgaagtcataaacagaaggcttctggccaagctgtggatgattttaagacacaaggtgtgaacatagaacagttcagagaattgaagaacacagtatcatacagtgggaaaaggaaagggcccgattcgttgtctgatggacctgcttgcaaaaggccagctctgttgcattcccaatttttgacaccacctcaaacaccaacgcccggggagagcatggaagatgttcatctcaatgaacccaaacaggagagcagtgctgatctgcttcagaacattatcaacattaagaatgaatgcagccccgtttccctgaacacagttcaagttagctggctgaaccccgtggtggtccctcagagctcccccgcagagcagtgtcaggacttccatggagggcaggtcttttctccacctcagaaatgccaaccattccaagtcaggggctcccaacaaatgatagaccaggcttccctgtaccagtattctccacagaaccagcatgtagagcagcagccacactacacccacaaaccaactctggaatacagtccttttcccatacctccccagtcccccgcttatgaaccaaacctctttgatggtccagaatcacagttttgcccaaaccaaagcttagtttcccttcttggtgatcaaagggaatctgagaatattgctaatcccatgcagacttcctccagtgttcagcagcaaaatgatgctcacttgcacagcttcagcatgatgcccagcagcgcctgtgaggccatggtggggcacgagatggcctctgactcttcaaacacttcactgccattctcaaacatgggaaatccaatgaacaccacacagttagggaaatcactttttcagtggcaggtggagcaggaagaaagcaaattggcaaatatttcccaagaccagtttctttcaaaggatgcagatggtgacacgttccttcatattgctgttgcccaagggagaagggcactttcctatgttcttgcaagaaagatgaatgcacttcacatgctggatattaaagagcacaatggacagagtgcctttcaggtggcagtggctgccaatcagcatctcattgtgcaggatctggtgaacatcggggcacaggtgaacaccacagactgctggggaagaacacctctgcatgtgtgtgctgagaagggccactcccaggtgcttcaggcgattcagaagggagcagtgggaagtaatcagtttgtggatcttgaggcaactaactatgatggcctgactccccttcactgtgcagtcatagcccacaatgctgtggtccatgaactccagagaaatcaacagcctcattcacctgaagttcaggagcttttactgaagaataagagtctggttgataccattaagtgcctaattcaaatgggagcagcggtggaagcgaaggatcgcaaaagtggccgcacagccctgcatttggcagctgaagaagcaaatctggaactcattcgcctctttttggagctgcccagttgcctgtcttttgtgaatgcaaaggcttacaatggcaacactgccctccatgttgctgccagcttgcagtatcggttgacacaattagatgctgtccgcctgttgatgaggaagggagcagacccaagtactcggaacttggagaacgaacagccagtgcatttggttcccgatggccctgtgggagaacagatccgacgtatcctgaagggaaagtccattcagcagagagctccaccgtattagctccattagcttggagcctggctagcaacactcactgtcagttaggcagtcctgatgtatctgtacatagaccatttgccttatattggcaaatgtaagttgtttctatgaaacaaacatatttagttcactattatatagtgggttatattaaaagaaaagaagaaaaatatctaatttctcttggcagatttgcatatttcatacccaggtatctgggatctagacatctgaatttgatctcaatggtaacattgccttcaattaacagtagcttttgagtaggaaaggactttgatttgtggcacaaaacattattaatatagctattgacagtttcaaagcaggtaaattgtaaatgtttctttaagaaaaagcatgtgaaaggaaaaaggtaaatacagcattgaggcttcatttggccttagtccctgggagttactggcgttggacaggcttcagtcattggactagatgaaaggtgtccatggttagaatttgatctttgcaaactgtatataattgttatttttgtccttaaaaatattgtacatacttggttgttaacatggtcatatttgaaatgtataagtccataaaatagaaaagaacaagtgaattgttgctatttaaaaaaattttacaattcttactaaggagtttttattgtgtaatcactaagtctttgtagataaagcagatggggagttacggagttgttcctttactggctgaaagatatattcgaattgtaaagatgctttttctcatgcattgaaattatacattatttgtagggaattgcatgcttttttttttttttctcccgagacagggtcttgctctggcgcccaggctggagtacagtggcatgatcttggctcacttcagccttgacttgggctcaagtgatcctcctacctgagccttctgagtaactggaactacaggtgtgcactcctcgcctggctaattttttattttttgtacaggcaggatcttgccaccttgcccaggctggtcttgaactcctgagctcatgccatctgcctgccttagtctcccaaaatgctgggattacaggagtgagccaccatgcccggctggcagttgcatggaagagaacacctctttatggcttaccctctagaatttctaatttatgtgttctgttgaaatttttgtttttttacctttattgaaacaacaaaaagtcagtattgaaacatatcttcctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaaccttttttttttaaaaaaaaaaaaaaagaaaatctcattagtgaacttatctttgcagctgagtacttaaattctttttaaaaagataccctttggattgatcacattgtttgacccagtatgtcttgtagacacgttagttataatcaccttgtatctctaaatatggtgtgatatgaaccagtccattcacattggaaaaactgatggttttaaataaactaattcactaataaaaaaaaaaaaaaaa

SEQ ID NO: 12 - protein, 718 aa, Homo sapiens
MIVDKLLDDSRGGEGLRDAAGGCGLMTSPLNLSYFYGASPPAAAPGACDASCSVLGPSAPGSPGSDSSDFSSASSVSSCGAVESRSRGGARAERQPVEPHMGVGRQQRGPFQGVRVKNSVKELLLHIRSHKQKASGQAVDDFKTQGVNIEQFRELKNTVSYSGKRKGPDSLSDGPACKRPALLHSQFLTPPQTPTPGESMEDVHLNEPKQESSADLLQNIINIKNECSPVSLNTVQVSWLNPVVVPQSSPAEQCQDFHGGQVFSPPQKCQPFQVRGSQQMIDQASLYQYSPQNQHVEQQPHYTHKPTLEYSPFPIPPQSPAYEPNLFDGPESQFCPNQSLVSLLGDQRESENIANPMQTSSSVQQQNDAHLHSFSMMPSSACEAMVGHEMASDSSNTSLPFSNMGNPMNTTQLGKSLFQWQVEQEESKLANISQDQFLSKDADGDTFLHIAVAQGRRALSYVLARKMNALHMLDIKEHNGQSAFQVAVAANQHLIVQDLVNIGAQVNTTDCWGRTPLHVCAEKGHSQVLQAIQKGAVGSNQFVDLEATNYDGLTPLHCAVIAHNAVVHELQRNQQPHSPEVQELLLKNKSLVDTIKCLIQMGAAVEAKDRKSGRTALHLAAEEANLELIRLFLELPSCLSFVNAKAYNGNTALHVAASLQYRLTQLDAVRLLMRKGADPSTRNLENEQPVHLVPDGPVGEQIRRILKGKSIQQRAPPY

SEQ ID NO: 13 - DNA, 3782 bp, Homo sapiens
ggcgagttcttagagaaaaaggctgcttagctgctgcttatcatgtaacctcaaaaggaaactgatcgtctttctcatgctgtcacgtacttgggttattatcgctgattacagctggaaacaattgatttgctcttacgtatttgtgtgacttgactcttcaaacacaaaggttaacaggaagatctcgagggccctggctgaacttcaccttttggctttcttggcctgatgctgaactctcgaggttgagccccatatgggggttggcaggcagcagagaggcccctttcaaggtgttcgggtaaagaactcagtgaaggaactcctgttgcacatccgaagtcataaacagaaggcttctggccaagctgtggatgattttaagacacaaggtgtgaacatagaacagttcagagaattgaagaacacagtatcatacagtgggaaaaggaaagggcccgattcgttgtctgatggacctgcttgcaaaaggccagctctgttgcattcccaatttttgacaccacctcaaacaccaacgcccggggagagcatggaagatgttcatctcaatgaacccaaacaggagagcagtgctgatctgcttcagaacattatcaacattaagaatgaatgcagccccgtttccctgaacacagttcaagttagctggctgaaccccgtggtggtccctcagagctcccccgcagagcagtgtcaggacttccatggagggcaggtcttttctccacctcagaaatgccaaccattccaagtcaggggctcccaacaaatgatagaccaggcttccctgtaccagtattctccacagaaccagcatgtagagcagcagccacactacacccacaaaccaactctggaatacagtccttttcccatacctccccagtcccccgcttatgaaccaaacctctttgatggtccagaatcacagttttgcccaaaccaaagcttagtttcccttcttggtgatcaaagggaatctgagaatattgctaatcccatgcagacttcctccagtgttcagcagcaaaatgatgctcacttgcacagcttcagcatgatgcccagcagcgcctgtgaggccatggtggggcacgagatggcctctgactcttcaaacacttcactgccattctcaaacatgggaaatccaatgaacaccacacagttagggaaatcactttttcagtggcaggtggagcaggaagaaagcaaattggcaaatatttcccaagaccagtttctttcaaaggatgcagatggtgacacgttccttcatattgctgttgcccaagggagaagggcactttcctatgttcttgcaagaaagatgaatgcacttcacatgctggatattaaagagcacaatggacagagtgcctttcaggtggcagtggctgccaatcagcatctcattgtgcaggatctggtgaacatcggggcacaggtgaacaccacagactgctggggaagaacacctctgcatgtgtgtgctgagaagggccactcccaggtgcttcaggcgattcagaagggagcagtgggaagtaatcagtttgtggatcttgaggcaactaactatgatggcctgactccccttcactgtgcagtcatagcccacaatgctgtggtccatgaactccagagaaatcaacagcctcattcacctgaagttcaggagcttttactgaagaataagagtctggttgataccattaagtgcctaattcaaatgggagcagcggtggaagcgaaggatcgcaaaagtggccgcacagccctgcatttggcagctgaagaagcaaatctggaactcattcgcctctttttggagctgcccagttgcctgtcttttgtgaatgcaaaggcttacaatggcaacactgccctccatgttgctgccagcttgcagtatcggttgacacaattagatgctgtccgcctgttgatgaggaagggagcagacccaagtactcggaacttggagaacgaacagccagtgcatttggttcccgatggccctgtgggagaacagatccgacgtatcctgaagggaaagtccattcagcagagagctccaccgtattagctccattagcttggagcctggctagcaacactcactgtcagttaggcagtcctgatgtatctgtacatagaccatttgccttatattggcaaatgtaagttgtttctatgaaacaaacatatttagttcactattatatagtgggttatattaaaagaaaagaagaaaaatatctaatttctcttggcagatttgcatatttcatacccaggtatctgggatctagacatctgaatttgatctcaatggtaacattgccttcaattaacagtagcttttgagtaggaaaggactttgatttgtggcacaaaacattattaatatagctattgacagtttcaaagcaggtaaattgtaaatgtttctttaagaaaaagcatgtgaaaggaaaaaggtaaatacagcattgaggcttcatttggccttagtccctgggagttactggcgttggacaggcttcagtcattggactagatgaaaggtgtccatggttagaatttgatctttgcaaactgtatataattgttatttttgtccttaaaaatattgtacatacttggttgttaacatggtcatatttgaaatgtataagtccataaaatagaaaagaacaagtgaattgttgctatttaaaaaaattttacaattcttactaaggagtttttattgtgtaatcactaagtctttgtagataaagcagatggggagttacggagttgttcctttactggctgaaagatatattcgaattgtaaagatgctttttctcatgcattgaaattatacattatttgtagggaattgcatgcttttttttttttttctcccgagacagggtcttgctctggcgcccaggctggagtacagtggcatgatcttggctcacttcagccttgacttgggctcaagtgatcctcctacctgagccttctgagtaactggaactacaggtgtgcactcctcgcctggctaattttttattttttgtacaggcaggatcttgccaccttgcccaggctggtcttgaactcctgagctcatgccatctgcctgccttagtctcccaaaatgctgggattacaggagtgagccaccatgcccggctggcagttgcatggaagagaacacctctttatggcttaccctctagaatttctaatttatgtgttctgttgaaatttttgtttttttacctttattgaaacaacaaaaagtcagtattgaaacatatcttcctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaaccttttttttttaaaaaaaaaaaaaaagaaaatctcattagtgaacttatctttgcagctgagtacttaaattctttttaaaaagataccctttggattgatcacattgtttgacccagtatgtcttgtagacacgttagttataatcaccttgtatctctaaatatggtgtgatatgaaccagtccattcacattggaaaaactgatggttttaaataaactaattcactaataaaaaaaaaaaaaaaa

SEQ ID NO: 14 - protein, 618 aa, Homo sapiens
MGVGRQQRGPFQGVRVKNSVKELLLHIRSHKQKASGQAVDDFKTQGVNIEQFRELKNTVSYSGKRKGPDSLSDGPACKRPALLHSQFLTPPQTPTPGESMEDVHLNEPKQESSADLLQNIINIKNECSPVSLNTVQVSWLNPVVVPQSSPAEQCQDFHGGQVFSPPQKCQPFQVRGSQQMIDQASLYQYSPQNQHVEQQPHYTHKPTLEYSPFPIPPQSPAYEPNLFDGPESQFCPNQSLVSLLGDQRESENIANPMQTSSSVQQQNDAHLHSFSMMPSSACEAMVGHEMASDSSNTSLPFSNMGNPMNTTQLGKSLFQWQVEQEESKLANISQDQFLSKDADGDTFLHIAVAQGRRALSYVLARKMNALHMLDIKEHNGQSAFQVAVAANQHLIVQDLVNIGAQVNTTDCWGRTPLHVCAEKGHSQVLQAIQKGAVGSNQFVDLEATNYDGLTPLHCAVIAHNAVVHELQRNQQPHSPEVQELLLKNKSLVDTIKCLIQMGAAVEAKDRKSGRTALHLAAEEANLELIRLFLELPSCLSFVNAKAYNGNTALHVAASLQYRLTQLDAVRLLMRKGADPSTRNLENEQPVHLVPDGPVGEQIRRILKGKSIQQRAPPY

SEQ ID NO: 15 - DNA, 3582 bp, Homo sapiens
gttgggtgggagcacaggcaccgggcaccatggggaaggccgagaactatgagctctactcggtggagctgggtcctggccctggcggggacatggctgccaagatgagcaagaagaagaaggcgggtggcgggggtggcaagaggaaggagaagctggagaacatgaagaaggagatggagattaacgaccaccagctgtcagtggcggagctggaacagaaataccagaccagtgccaccaagggcctctctgcgagcctggctgctgagctgctgctgcgggatgggcccaacgcactgcggccaccacggggcaccccagagtacgtcaagttcgcgaggcagctggccgggggcctgcagtgcctcatgtgggttgccgccgccatctgcctcatcgcctttgccatccaggctagtgagggggacctcaccaccgacgacaatctgtacctggcaatcgctctcattgctgtggttgtcgtcaccggctgctttggctactaccaggaattcaagagcaccaacatcatcgccagctttaagaaccttgtgccacagcaagccactgtcatccgcgatggagacaaattccagatcaacgctgaccaactggtggtgggcgacctggtggagatgaaaggtggggacagagtgcccgccgacatccgcatcctggcggcccagggctgcaaggtggacaactcctcgctgacaggggagtctgagccacagacccgctcacccgagtgcacgcacgagagccctctggagacccgcaacatcgccttcttctccaccatgtgccttgagggcaccgtgcagggcctggtggtgaacacgggcgaccgcaccatcattgggcgcatcgcatcgctggcgtcgggggtggaaaacgagaagacacccatcgctatcgagatcgagcattttgtggacatcatcgcgggcctggccattctcttcggtgccacattttttattgtggccatgtgcattggctacaccttcctgcgggccatggtcttcttcatggccatcgtggtggcctatgtgcctgaggggctgctggccactgtcacagtctgcctgtccctgacagccaagcgcctggccagtaagaactgcgtggtcaagaacctggaggcggtggagacattgggctccacttcggtgatctgctcggacaagacagggactctcactcagaaccgcatgactgtgtcccatctgtggtttgacaaccacatccacacagctgacaccacggaagaccagtcagggcagacgtttgaccagtcctcggagacgtggcgggcgctgtgccgggtgctcaccctgtgcaaccgcgccgccttcaagtccggccaggatgcagtgcctgtgcccaagcgcatcgtgattggagacgcatcggagacggcgctgctcaagttctcggagctgacgctgggcaacgccatgggctaccgggaccgcttcccaaaagtctgcgagatacccttcaactccaccaacaagttccagctgtccatccatacgctggaggacccgcgggacccgcgacacttgctggtgatgaagggcgcccccgagcgcgtgctggagcgctgcagctccatccttatcaagggccaggagctgccgctggacgagcagtggcgcgaggccttccagaccgcctacctcagcctgggaggcctgggcgaacgcgtgctcggcttctgccagctctacctgaatgagaaggactacccgcctggctatgccttcgacgtagaggccatgaactttccatctagcggcctctgctttgcgggacttgtatccatgattgacccaccccgggccaccgtccctgatgctgtgctcaagtgtcgcaccgcaggcatccgggtgatcatggtaacgggtgaccaccccatcaccgccaaggccattgcagccagtgtgggcatcatctcggaaggcagcgagacagtggaggacatcgctgcccgcctccgtgtgcccgtagaccaggttaatcgcaaggatgcccgtgcctgtgtgatcaatggcatgcagctgaaggacatggacccatcggaactggtcgaggccctgcgcacccaccccgagatggtgtttgcgcgcaccagcccccagcagaagctggtgatcgtggagagctgccagcggctgggtgcgattgtggccgtcacgggggatggtgtgaatgactccccagctctgaagaaggcagacatcggagtagccatgggcatcgctggctcagatgctgccaaaaatgcagctgacatgatcctgctggatgacaactttgcctccattgtgacaggcgtggagcagggtcgactgatcttcgacaacctgaagaagtctattgcctacacattgaccaagaacatcccagagctgacaccctacctcatctacatcaccgtcagcgtgcccctgcccctcgggtgcatcaccatcctcttcatcgaactctgcactgacattttcccatctgtgtccctggcatatgaaaaggccgagagtgacatcatgcacctgcgtccacgcaacccaaagcgtgacagattggtcaacgagcccctggctgcctactcctacttccagattggtgccattcagtcctttgctggcttcactgactacttcacggcaatggcccaggagggctggttcccactgctgtgcgtggggctgcgggcgcagtgggaggaccaccacctacaagatctgcaggacagctacggccaggagtggacattcgggcagcgcctgtaccagcagtacacctgctacaccgtgttcttcatcagcattgaggtgtgccagatcgccgatgtcctcatccgcaagacgcgccgtctctctgccttccagcaaggcttcttcaggaataagatcctggtgatcgccatcgtgttccaggtctgcatcggctgcttcctgtgctactgccccggcatgcccaacatcttcaacttcatgcccattcggttccagtggtggctggtccccctgccctacggcatcctcatcttcgtctatgatgagatccggaagcttggagttcgctgttgcccagggagctggtgggaccaggaactctactattagagggacgactgccttcaagcatccctgcaactgccacagcaggtgggggcagggctcgtgggaccctctggacagccaccaagatatctgagcaaccaagagtcccagccccaccagtatctgcttctgtagcccacggcaccccaaacttggagggacctgcccactcccctcccccattcccaaggttcgcacctcctggagcagcagcgcctgggcagtcctctgggctggcctcgggaaagccgccacctgtggtggcggtggggctctgacagggagtacagctgaccgcttctggagggtgtttctgttcttaggactccagtccaggctggacggctgcctgagggcccttcgttaaagacacgcttgtgtcctgggcgatggtaataaaaccagctcatgctgactgtgctgtaaaaaaaaaaaaaaaaaaaaaaaa

SEQ ID NO: 16 - protein, 1035 aa, Homo sapiens
MGKAENYELYSVELGPGPGGDMAAKMSKKKKAGGGGGKRKEKLENMKKEMEINDHQLSVAELEQKYQTSATKGLSASLAAELLLRDGPNALRPPRGTPEYVKFARQLAGGLQCLMWVAAAICLIAFAIQASEGDLTTDDNLYLAIALIAVVVVTGCFGYYQEFKSTNIIASFKNLVPQQATVIRDGDKFQINADQLVVGDLVEMKGGDRVPADIRILAAQGCKVDNSSLTGESEPQTRSPECTHESPLETRNIAFFSTMCLEGTVQGLVVNTGDRTIIGRIASLASGVENEKTPIAIEIEHFVDIIAGLAILFGATFFIVAMCIGYTFLRAMVFFMAIVVAYVPEGLLATVTVCLSLTAKRLASKNCVVKNLEAVETLGSTSVICSDKTGTLTQNRMTVSHLWFDNHIHTADTTEDQSGQTFDQSSETWRALCRVLTLCNRAAFKSGQDAVPVPKRIVIGDASETALLKFSELTLGNAMGYRDRFPKVCEIPFNSTNKFQLSIHTLEDPRDPRHLLVMKGAPERVLERCSSILIKGQELPLDEQWREAFQTAYLSLGGLGERVLGFCQLYLNEKDYPPGYAFDVEAMNFPSSGLCFAGLVSMIDPPRATVPDAVLKCRTAGIRVIMVTGDHPITAKAIAASVGIISEGSETVEDIAARLRVPVDQVNRKDARACVINGMQLKDMDPSELVEALRTHPEMVFARTSPQQKLVIVESCQRLGAIVAVTGDGVNDSPALKKADIGVAMGIAGSDAAKNAADMILLDDNFASIVTGVEQGRLIFDNLKKSIAYTLTKNIPELTPYLIYITVSVPLPLGCITILFIELCTDIFPSVSLAYEKAESDIMHLRPRNPKRDRLVNEPLAAYSYFQIGAIQSFAGFTDYFTAMAQEGWFPLLCVGLRAQWEDHHLQDLQDSYGQEWTFGQRLYQQYTCYTVFFISIEVCQIADVLIRKTRRLSAFQQGFFRNKILVIAIVFQVCIGCFLCYCPGMPNIFNFMPIRFQWWLVPLPYGILIFVYDEIRKLGVRCCPGSWWDQELYY

SEQ ID NO: 17 - DNA, 2066 bp, Homo sapiens
agcatttaatcatgaggcactccaaagaatatgagatagaaggggaggggtaggaggagagggaggagggtaggaagacagtttgcattcttgcaacattaaaccaaagggacttggagtgcagatggcatccttcggttcttccagacaagctgcaagacgctgaccatggccaagatggagctctcgaaggccttctctggccagcggacactcctatctgccatcctcagcatgctatcactcagcttctccacaacatccctgctcagcaactactggtttgtgggcacacagaaggtgcccaagcccctgtgcgagaaaggtctggcagccaagtgctttgacatgccagtgtccctggatggagataccaacacatccacccaggaggtggtacaatacaactgggagactggggatgaccggttctccttccggagcttccggagtggcatgtggctatcctgtgaggaaactgtggaagaaccaggggagaggtgccgaagtttcattgaacttacaccaccagccaagagaggtgagaaaggactactggaatttgccacgttgcaaggcccatgtcaccccactctccgatttggagggaagcggttgatggagaaggcttccctcccctcccctcccttggggctttgtggcaaaaatcctatggttatccctgggaacgcagatcacctacatcggacttcaattcatcagcttcctcctgctactaacagacttgctactcactgggaaccctgcctgtgggctcaaactgagcgcctttgctgctgtttcctctgtcctgtcaggtctcctggggatggtggcccacatgatgtattcacaagtcttccaagcgactgtcaacttgggtccagaagactggagaccacatgtttggaattatggctgggccttctacatggcctggctctccttcacctgctgcatggcgtcggctgtcaccaccttcaacacgtacaccaggatggtgctggagttcaagtgcaagcatagtaagagcttcaaggaaaacccgaactgcctaccacatcaccatcagtgtttccctcggcggctgtcaagtgcagcccccaccgtgggtcctttgaccagctaccaccagtatcataatcagcccatccactctgtctctgagggagtcgacttctactccgagctgcggaacaagggatttcaaagaggggccagccaggagctgaaagaagcagttaggtcatctgtagaggaagagcagtgttaggagttaagcgggtttggggagtaggcttgagccctaccttacacgtctgctgattatcaacatgtgcttaagccaacatccgtctcttgagcatggtttttagaggctacgaataaggctatgaataagggttatctttaagtcctaagggattcctgggtgccactgctctcttttcctctacagctccatcttgtttcacccaccccacatctcacacatccagaattcccttctttactgatagtttctgtgccaggttctgggctaaaccatggagataaaaagaagagtaaaatacacttcccgaccttaaggatctgatacatctagttattggtctggaaattggagaaatggaggtgagcaaattaacggtagtgtgactgtggctcaactttccatggtacgcgcgaattgttctggaagtcacagatgggaagacagaagcgcccctgagtgctttgtcatatagcacagaagtcctccacactgcctgggacagtgtcctgcatccacacagtaggtacccagtaaaagtgttattaaatattgtcccaggagcccttaggagagtggtttaacatgagaaggcgccaacagcacacagggtcagggtcacttctagaaatagcccaccaaggctattcagattggactgtggttcccttctggacagtcacaccagggacttcccagtgaacaagggggcataggctcctctctggatcccaccttttcatcccttctgagaaatgtaccctattatttccttcataaagaatgccaccca

SEQ ID NO: 18 - protein, 285 aa, Homo sapiens
MAKMELSKAFSGQRTLLSAILSMLSLSFSTTSLLSNYWFVGTQKVPKPLCEKGLAAKCFDMPVSLDGDTNTSTQEVVQYNWETGDDRFSFRSFRSGMWLSCEETVEEPGERCRSFIELTPPAKRGEKGLLEFATLQGPCHPTLRFGGKRLMEKASLPSPPLGLCGKNPMVIPGNADHLHRTSIHQLPPATNRLATHWEPCLWAQTERLCCCFLCPVRSPGDGGPHDVFTSLPSDCQLGSRRLETTCLELWLGLLHGLALLHLLHGVGCHHLQHVHQDGAGVQVQA

SEQ ID NO: 19 - DNA, 1942 bp, Homo sapiens
agcatttaatcatgaggcactccaaagaatatgagatagaaggggaggggtaggaggagagggaggagggtaggaagacagtttgcattcttgcaacattaaaccaaagggacttggagtgcagatggcatccttcggttcttccagacaagctgcaagacgctgaccatggccaagatggagctctcgaaggccttctctggccagcggacactcctatctgccatcctcagcatgctatcactcagcttctccacaacatccctgctcagcaactactggtttgtgggcacacagaaggtgcccaagcccctgtgcgagaaaggtctggcagccaagtgctttgacatgccagtgtccctggatggagataccaacacatccacccaggaggtggtacaatacaactgggagactggggatgaccggttctccttccggagcttccggagtggcatgtggctatcctgtgaggaaactgtggaagaaccaggggagaggtgccgaagtttcattgaacttacaccaccagccaagagagaaatcctatggttatccctgggaacgcagatcacctacatcggacttcaattcatcagcttcctcctgctactaacagacttgctactcactgggaaccctgcctgtgggctcaaactgagcgcctttgctgctgtttcctctgtcctgtcaggtctcctggggatggtggcccacatgatgtattcacaagtcttccaagcgactgtcaacttgggtccagaagactggagaccacatgtttggaattatggctgggccttctacatggcctggctctccttcacctgctgcatggcgtcggctgtcaccaccttcaacacgtacaccaggatggtgctggagttcaagtgcaagcatagtaagagcttcaaggaaaacccgaactgcctaccacatcaccatcagtgtttccctcggcggctgtcaagtgcagcccccaccgtgggtcctttgaccagctaccaccagtatcataatcagcccatccactctgtctctgagggagtcgacttctactccgagctgcggaacaagggatttcaaagaggggccagccaggagctgaaagaagcagttaggtcatctgtagaggaagagcagtgttaggagttaagcgggtttggggagtaggcttgagccctaccttacacgtctgctgattatcaacatgtgcttaagccaacatccgtctcttgagcatggtttttagaggctacgaataaggctatgaataagggttatctttaagtcctaagggattcctgggtgccactgctctcttttcctctacagctccatcttgtttcacccaccccacatctcacacatccagaattcccttctttactgatagtttctgtgccaggttctgggctaaaccatggagataaaaagaagagtaaaatacacttcccgaccttaaggatctgatacatctagttattggtctggaaattggagaaatggaggtgagcaaattaacggtagtgtgactgtggctcaactttccatggtacgcgcgaattgttctggaagtcacagatgggaagacagaagcgcccctgagtgctttgtcatatagcacagaagtcctccacactgcctgggacagtgtcctgcatccacacagtaggtacccagtaaaagtgttattaaatattgtcccaggagcccttaggagagtggtttaacatgagaaggcgccaacagcacacagggtcagggtcacttctagaaatagcccaccaaggctattcagattggactgtggttcccttctggacagtcacaccagggacttcccagtgaacaagggggcataggctcctctctggatcccaccttttcatcccttctgagaaatgtaccctattatttccttcataaagaatgccaccca

SEQ ID NO: 20 - protein, 326 aa, Homo sapiens
MAKMELSKAFSGQRTLLSAILSMLSLSFSTTSLLSNYWFVGTQKVPKPLCEKGLAAKCFDMPVSLDGDTNTSTQEVVQYNWETGDDRFSFRSFRSGMWLSCEETVEEPGERCRSFIELTPPAKREILWLSLGTQITYIGLQFISFLLLLTDLLLTGNPACGLKLSAFAAVSSVLSGLLGMVAHMMYSQVFQATVNLGPEDWRPHVWNYGWAFYMAWLSFTCCMASAVTTFNTYTRMVLEFKCKHSKSFKENPNCLPHHHQCFPRRLSSAAPTVGPLTSYHQYHNQPIHSVSEGVDFYSELRNKGFQRGASQELKEAVRSSVEEEQC

SEQ ID NO: 21 - DNA, 2179 bp, Homo sapiens
ggctggtgtctggccctcaggacatcctctccaatccaccacacaccaccttacccctctgctggcaagaggggacctgattcatcctcacgctaaacactcattctacccaactgattgagacagaacagaagataaactgaaacttctctgccttcccgctgcaagagtgaatgagcgatccctctcaactgactcaaaatgtttgcctcacccaggagatggagctctcgaaggccttctctggccagcggacactcctatctgccatcctcagcatgctatcactcagcttctccacaacatccctgctcagcaactactggtttgtgggcacacagaaggtgcccaagcccctgtgcgagaaaggtctggcagccaagtgctttgacatgccagtgtccctggatggagataccaacacatccacccaggaggtggtacaatacaactgggagactggggatgaccggttctccttccggagcttccggagtggcatgtggctatcctgtgaggaaactgtggaagaaccagcactgctccatccccagtcctggaaacaatttagagcccttcggtccagtggtacagcggcagcaaaaggggagaggtgccgaagtttcattgaacttacaccaccagccaagagaggtgagaaaggactactggaatttgccacgttgcaaggcccatgtcaccccactctccgatttggagggaagcggttgatggagaaggcttccctcccctcccctcccttggggctttgtggcaaaaatcctatggttatccctgggaacgcagatcacctacatcggacttcaattcatcagcttcctcctgctactaacagacttgctactcactgggaaccctgcctgtgggctcaaactgagcgcctttgctgctgtttcctctgtcctgtcaggtctcctggggatggtggcccacatgatgtattcacaagtcttccaagcgactgtcaacttgggtccagaagactggagaccacatgtttggaattatggctgggccttctacatggcctggctctccttcacctgctgcatggcgtcggctgtcaccaccttcaacacgtacaccaggatggtgctggagttcaagtgcaagcatagtaagagcttcaaggaaaacccgaactgcctaccacatcaccatcagtgtttccctcggcggctgtcaagtgcagcccccaccgtgggtcctttgaccagctaccaccagtatcataatcagcccatccactctgtctctgagggagtcgacttctactccgagctgcggaacaagggatttcaaagaggggccagccaggagctgaaagaagcagttaggtcatctgtagaggaagagcagtgttaggagttaagcgggtttggggagtaggcttgagccctaccttacacgtctgctgattatcaacatgtgcttaagccaacatccgtctcttgagcatggtttttagaggctacgaataaggctatgaataagggttatctttaagtcctaagggattcctgggtgccactgctctcttttcctctacagctccatcttgtttcacccaccccacatctcacacatccagaattcccttctttactgatagtttctgtgccaggttctgggctaaaccatggagataaaaagaagagtaaaatacacttcccgaccttaaggatctgatacatctagttattggtctggaaattggagaaatggaggtgagcaaattaacggtagtgtgactgtggctcaactttccatggtacgcgcgaattgttctggaagtcacagatgggaagacagaagcgcccctgagtgctttgtcatatagcacagaagtcctccacactgcctgggacagtgtcctgcatccacacagtaggtacccagtaaaagtgttattaaatattgtcccaggagcccttaggagagtggtttaacatgagaaggcgccaacagcacacagggtcagggtcacttctagaaatagcccaccaaggctattcagattggactgtggttcccttctggacagtcacaccagggacttcccagtgaacaagggggcataggctcctctctggatcccaccttttcatcccttctgagaaatgtaccctattatttccttcataaagaatgccaccca

SEQ ID NO: 22 - protein, 321 aa, Homo sapiens
MSDPSQLTQNVCLTQEMELSKAFSGQRTLLSAILSMLSLSFSTTSLLSNYWFVGTQKVPKPLCEKGLAAKCFDMPVSLDGDTNTSTQEVVQYNWETGDDRFSFRSFRSGMWLSCEETVEEPALLHPQSWKQFRALRSSGTAAAKGERCRSFIELTPPAKRGEKGLLEFATLQGPCHPTLRFGGKRLMEKASLPSPPLGLCGKNPMVIPGNADHLHRTSIHQLPPATNRLATHWEPCLWAQTERLCCCFLCPVRSPGDGGPHDVFTSLPSDCQLGSRRLETTCLELWLGLLHGLALLHLLHGVGCHHLQHVHQDGAGVQVQA

SEQ ID NO: 23 - DNA, 2055 bp, Homo sapiens
ggctggtgtctggccctcaggacatcctctccaatccaccacacaccaccttacccctctgctggcaagaggggacctgattcatcctcacgctaaacactcattctacccaactgattgagacagaacagaagataaactgaaacttctctgccttcccgctgcaagagtgaatgagcgatccctctcaactgactcaaaatgtttgcctcacccaggagatggagctctcgaaggccttctctggccagcggacactcctatctgccatcctcagcatgctatcactcagcttctccacaacatccctgctcagcaactactggtttgtgggcacacagaaggtgcccaagcccctgtgcgagaaaggtctggcagccaagtgctttgacatgccagtgtccctggatggagataccaacacatccacccaggaggtggtacaatacaactgggagactggggatgaccggttctccttccggagcttccggagtggcatgtggctatcctgtgaggaaactgtggaagaaccagcactgctccatccccagtcctggaaacaatttagagcccttcggtccagtggtacagcggcagcaaaaggggagaggtgccgaagtttcattgaacttacaccaccagccaagagagaaatcctatggttatccctgggaacgcagatcacctacatcggacttcaattcatcagcttcctcctgctactaacagacttgctactcactgggaaccctgcctgtgggctcaaactgagcgcctttgctgctgtttcctctgtcctgtcaggtctcctggggatggtggcccacatgatgtattcacaagtcttccaagcgactgtcaacttgggtccagaagactggagaccacatgtttggaattatggctgggccttctacatggcctggctctccttcacctgctgcatggcgtcggctgtcaccaccttcaacacgtacaccaggatggtgctggagttcaagtgcaagcatagtaagagcttcaaggaaaacccgaactgcctaccacatcaccatcagtgtttccctcggcggctgtcaagtgcagcccccaccgtgggtcctttgaccagctaccaccagtatcataatcagcccatccactctgtctctgagggagtcgacttctactccgagctgcggaacaagggatttcaaagaggggccagccaggagctgaaagaagcagttaggtcatctgtagaggaagagcagtgttaggagttaagcgggtttggggagtaggcttgagccctaccttacacgtctgctgattatcaacatgtgcttaagccaacatccgtctcttgagcatggtttttagaggctacgaataaggctatgaataagggttatctttaagtcctaagggattcctgggtgccactgctctcttttcctctacagctccatcttgtttcacccaccccacatctcacacatccagaattcccttctttactgatagtttctgtgccaggttctgggctaaaccatggagataaaaagaagagtaaaatacacttcccgaccttaaggatctgatacatctagttattggtctggaaattggagaaatggaggtgagcaaattaacggtagtgtgactgtggctcaactttccatggtacgcgcgaattgttctggaagtcacagatgggaagacagaagcgcccctgagtgctttgtcatatagcacagaagtcctccacactgcctgggacagtgtcctgcatccacacagtaggtacccagtaaaagtgttattaaatattgtcccaggagcccttaggagagtggtttaacatgagaaggcgccaacagcacacagggtcagggtcacttctagaaatagcccaccaaggctattcagattggactgtggttcccttctggacagtcacaccagggacttcccagtgaacaagggggcataggctcctctctggatcccaccttttcatcccttctgagaaatgtaccctattatttccttcataaagaatgccaccca

SEQ ID NO: 24 - protein, 362 aa, Homo sapiens
MSDPSQLTQNVCLTQEMELSKAFSGQRTLLSAILSMLSLSFSTTSLLSNYWFVGTQKVPKPLCEKGLAAKCFDMPVSLDGDTNTSTQEVVQYNWETGDDRFSFRSFRSGMWLSCEETVEEPALLHPQSWKQFRALRSSGTAAAKGERCRSFIELTPPAKREILWLSLGTQITYIGLQFISFLLLLTDLLLTGNPACGLKLSAFAAVSSVLSGLLGMVAHMMYSQVFQATVNLGPEDWRPHVWNYGWAFYMAWLSFTCCMASAVTTFNTYTRMVLEFKCKHSKSFKENPNCLPHHHQCFPRRLSSAAPTVGPLTSYHQYHNQPIHSVSEGVDFYSELRNKGFQRGASQELKEAVRSSVEEEQC

SEQ ID NO: 25 - DNA, 3183 bp, Homo sapiens
cgaggctcctcctctgtggatggtcactgcccctccaccaggcttcctgctggaggagtttccttcccagccaggccggcccagaagccagatggtcccgggacaggcccagccccagagcccagagatgctgctgctgcccctgctgctgcccgtgctgggggcggggtccctgaacaaggatcccagttacagtcttcaagtgcagaggcaggtgccggtgccggagggcctgtgtgtcatcgtgtcttgcaacctctcctacccccgggatggctgggacgagtctactgctgcttatggctactggttcaaaggacggaccagcccaaagacgggtgctcctgtggccactaacaaccagagtcgagaggtggaaatgagcacccgggaccgattccagctcactggggatcccggcaaagggagctgctccttggtgatcagagacgcgcagagggaggatgaggcatggtacttctttcgggtggagagaggaagccgtgtgagacatagtttcctgagcaatgcgttctttctaaaagtaacagccctgactaagaagcctgatgtctacatccccgagaccctggagcccgggcagccggtgacggtcatctgtgtgtttaactgggctttcaagaaatgtccagccccttctttctcctggacgggggctgccctctcccctagaagaaccagaccaagcacctcccacttctcagtgctcagcttcacgcccagcccccaggaccacgacaccgacctcacctgccatgtggacttctccagaaagggtgtgagcgcacagaggaccgtccgactccgtgtggcctatgcccccaaagaccttattatcagcatttcacatgacaacacgtcagccctggaactccagggaaacgtcatatatctggaagttcagaaaggccagttcctgcggctcctctgtgctgctgacagccagccccctgccacgctgagctgggtcctgcaggacagagtcctctcctcgtcccacccctggggccccagaaccctggggctggagctgcgtggggtaagggccggggattcagggcgctacacctgccgagcggagaacaggcttggctcccagcagcaagccctggacctctctgtgcagtatcctccagagaacctgagagtgatggtttcccaagcaaacaggacagtcctggaaaacctcgggaacggcacatccctcccggtcctggagggccaaagcctgcgcctggtctgtgtcacccacagcagccccccagccaggctgagctggacccggtggggacagaccgtgggcccctcccagccctcagaccccggggtcctggagctgccacccattcaaatggagcacgaaggagagttcacctgccacgctcagcaccctctgggctcccagcacgtctctctcagcctctccgtgcactaccctccacagctgctgggcccctcctgctcctgggaggctgagggtctgcactgcagctgctcctcccaggccagcccggccccctctctgcgctggtggcttggggaggagctgctggaggggaacagcagtcagggctccttcgaggtcacccccagctcagccgggccctgggccaacagctccctgagcctccatggagggctcagctccggcctcaggctccgctgtaaggcctggaacgtccacggggcccagagtggctctgtcttccagctgctaccagggaagctggagcatgggggaggacttggcctgggggctgccctgggagctggcgtcgctgccctgctcgctttctgttcctgccttgtcgtcttcagggtgaagatctgcaggaaggaagctcgcaagagggcagcagctgagcaggacgtgccctccaccctgggacccatctcccagggtcaccagcatgaatgctcggcaggcagctcccaagaccacccgcccccaggtgcagccacctacaccccggggaagggggaagagcaggagctccactatgcctccctcagcttccagggcctgaggctctgggagcctgcggaccaggaggcccccagcaccaccgagtactcggagatcaagatccacacaggacagcccctgaggggcccaggctttgggcttcaattggagagggagatgtcagggatggttccaaagtgaagaggtctccatggcaacaggacaccagcaagtgtgtgggagtcgcactggtgtgacggccagaactggactcagatttcagccccatccccaatgaagagcttgagtttgaagattatactttttttgagacagggtctgactctgtcctccaggccagagtccagtggtgcaatctcagctcactgtagcctcaacctgccaggttgaagtgagcctcccatttcagcctcccaagtagctgggactacaattgtgagccaccatgccaggctcattgttatatttttagtagagacagggttttgccatgtttccctggctggtctcagactcctgggctcaagcaatctgcccgcctctgcctcccaaagtgctgggattacagacgtgagccaccacagctggctgaagattatactttcaattcagagcgagtttgaagatgacactttgaggcatcgtgtctatggttcattactacagaagcttctctggatgtgtaaagcacaggaaaccaggcagaggaggcacagggtgctctccagaacgagaagccagctcctggagttgtttgctgcaactgccattccccgttgatgaccatgctcttccttcagaagagggagagtgagaggaccaagtccaagtggttcccatttgaacatttaaaaaaaaaaaaaaggctgggcatggtggctcacgcctgtaatctcaacactttgggaggctgaagtgggtggatcacaagtcaggagttcaagaccagcctgggcaagatggtgaaaccccatctctactaaaaatacaaaaattagccgggcatggtggcgggcgcctaaaatcccagctactcgggagactaggcagagaattggttgaacccgggaggtggaggttgcagtgagccgagatcgtcccactgcactccagcctgggcaacagagtgagactctgtttctaaataaataaatgaaaaaaaaaaaaaaaaaaaa

SEQ ID NO: 26 - protein, 698 aa, Homo sapiens
MVPGQAQPQSPEMLLLPLLLPVLGAGSLNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGLGAALGAGVAALLAFCSCLVVFRVKICRKEARKRAAAEQDVPSTLGPISQGHQHECSAGSSQDHPPPGAATYTPGKGEEQELHYASLSFQGLRLWEPADQEAPSTTEYSEIKIHTGQPLRGPGFGLQLEREMSGMVPK

SEQ ID NO: 27 - DNA, 2895 bp, Homo sapiens
cgaggctcctcctctgtggatggtcactgcccctccaccaggcttcctgctggaggagtttccttcccagccaggccggcccagaagccagatggtcccgggacaggcccagccccagagcccagagatgctgctgctgcccctgctgctgcccgtgctgggggcggggtccctgaacaaggatcccagttacagtcttcaagtgcagaggcaggtgccggtgccggagggcctgtgtgtcatcgtgtcttgcaacctctcctacccccgggatggctgggacgagtctactgctgcttatggctactggttcaaaggacggaccagcccaaagacgggtgctcctgtggccactaacaaccagagtcgagaggtggaaatgagcacccgggaccgattccagctcactggggatcccggcaaagggagctgctccttggtgatcagagacgcgcagagggaggatgaggcatggtacttctttcgggtggagagaggaagccgtgtgagacatagtttcctgagcaatgcgttctttctaaaagtaacagccctgactaagaagcctgatgtctacatccccgagaccctggagcccgggcagccggtgacggtcatctgtgtgtttaactgggctttcaagaaatgtccagccccttctttctcctggacgggggctgccctctcccctagaagaaccagaccaagcacctcccacttctcagtgctcagcttcacgcccagcccccaggaccacgacaccgacctcacctgccatgtggacttctccagaaagggtgtgagcgcacagaggaccgtccgactccgtgtggcctatgcccccaaagaccttattatcagcatttcacatgacaacacgtcagccctggaactccagggaaacgtcatatatctggaagttcagaaaggccagttcctgcggctcctctgtgctgctgacagccagccccctgccacgctgagctgggtcctgcaggacagagtcctctcctcgtcccacccctggggccccagaaccctggggctggagctgcgtggggtaagggccggggattcagggcgctacacctgccgagcggagaacaggcttggctcccagcagcaagccctggacctctctgtgcagtatcctccagagaacctgagagtgatggtttcccaagcaaacaggacagtcctggaaaacctcgggaacggcacatccctcccggtcctggagggccaaagcctgcgcctggtctgtgtcacccacagcagccccccagccaggctgagctggacccggtggggacagaccgtgggcccctcccagccctcagaccccggggtcctggagctgccacccattcaaatggagcacgaaggagagttcacctgccacgctcagcaccctctgggctcccagcacgtctctctcagcctctccgtgcactggaagctggagcatgggggaggacttggcctgggggctgccctgggagctggcgtcgctgccctgctcgctttctgttcctgccttgtcgtcttcagggtgaagatctgcaggaaggaagctcgcaagagggcagcagctgagcaggacgtgccctccaccctgggacccatctcccagggtcaccagcatgaatgctcggcaggcagctcccaagaccacccgcccccaggtgcagccacctacaccccggggaagggggaagagcaggagctccactatgcctccctcagcttccagggcctgaggctctgggagcctgcggaccaggaggcccccagcaccaccgagtactcggagatcaagatccacacaggacagcccctgaggggcccaggctttgggcttcaattggagagggagatgtcagggatggttccaaagtgaagaggtctccatggcaacaggacaccagcaagtgtgtgggagtcgcactggtgtgacggccagaactggactcagatttcagccccatccccaatgaagagcttgagtttgaagattatactttttttgagacagggtctgactctgtcctccaggccagagtccagtggtgcaatctcagctcactgtagcctcaacctgccaggttgaagtgagcctcccatttcagcctcccaagtagctgggactacaattgtgagccaccatgccaggctcattgttatatttttagtagagacagggttttgccatgtttccctggctggtctcagactcctgggctcaagcaatctgcccgcctctgcctcccaaagtgctgggattacagacgtgagccaccacagctggctgaagattatactttcaattcagagcgagtttgaagatgacactttgaggcatcgtgtctatggttcattactacagaagcttctctggatgtgtaaagcacaggaaaccaggcagaggaggcacagggtgctctccagaacgagaagccagctcctggagttgtttgctgcaactgccattccccgttgatgaccatgctcttccttcagaagagggagagtgagaggaccaagtccaagtggttcccatttgaacatttaaaaaaaaaaaaaaggctgggcatggtggctcacgcctgtaatctcaacactttgggaggctgaagtgggtggatcacaagtcaggagttcaagaccagcctgggcaagatggtgaaaccccatctctactaaaaatacaaaaattagccgggcatggtggcgggcgcctaaaatcccagctactcgggagactaggcagagaattggttgaacccgggaggtggaggttgcagtgagccgagatcgtcccactgcactccagcctgggcaacagagtgagactctgtttctaaataaataaatgaaaaaaaaaaaaaaaaaaaa

SEQ ID NO: 28 - protein, 602 aa, Homo sapiens
MVPGQAQPQSPEMLLLPLLLPVLGAGSLNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHWKLEHGGGLGLGAALGAGVAALLAFCSCLVVFRVKICRKEARKRAAAEQDVPSTLGPISQGHQHECSAGSSQDHPPPGAATYTPGKGEEQELHYASLSFQGLRLWEPADQEAPSTTEYSEIKIHTGQPLRGPGFGLQLEREMSGMVPK

(19) United States
(12) Patent Application Publication
(10) Pub. No.: US 2012/0264639 A1
(43) Pub. Date: Oct. 18, 2012

(54) METHODS AND COMPOSITIONS FOR PREDICTING SURVIVAL IN SUBJECTS WITH CANCER

(76) Inventor: Jen Jen Yeh, Chapel Hill, NC (US)

(21) Appl. No.: 13/505,172

(22) PCT Filed: Nov. 4, 2010
(86) PCT No.: PCT/US10/55435

§ 371 Date: Jun. 22, 2012

Related U.S. Application Data
(60) Provisional application No. 61/280,470, filed on Nov. 4, 2009.

(51) Int. Cl. C40B 30/04; G01N 21/64; C40B 40/10; G01N 21/78; C40B 40/06; C12Q 1/68; G01N 33/574

(52) U.S. Cl. 506/9; 435/6.11; 435/6.12; 435/7.1; 435/7.9; 506/16; 506/18

(57) ABSTRACT

Methods for generating a prognostic signature for a subject with pancreatic ductal adenocarcinoma (PDAC) are disclosed. In some embodiments, the methods include determining expression levels for one or more genes selected from among Fos B, KLF6, NFKBIZ, ATP4A, GSG1, and SIGLEC11 in PDAC cells obtained from the subject, wherein the determining provides a prognostic signature for the subject. Also disclosed are methods for assessing risk of an adverse outcome of a subject with pancreatic ductal adenocarcinoma (PDAC), methods for predicting a clinical outcome of a treatment in a subject diagnosed with pancreatic ductal adenocarcinoma (PDAC), methods for predicting a positive or a negative clinical response of a subject with pancreatic ductal adenocarcinoma (PDAC) to a treatment, and arrays for use in the disclosed methods.

### CROSS REFERENCE TO RELATED APPLICATION

This application claims the benefit of U.S. Provisional Patent Application Ser. No. 61/280,470, filed Nov. 4, 2009; the disclosure of which is incorporated herein by reference in its entirety.

### GOVERNMENT INTEREST

This invention was made with United States government support under Grant No. CA106991 awarded by National Institutes of Health of the United States. The United States government has certain rights in the invention.

### TECHNICAL FIELD

The presently disclosed subject matter relates to a gene expression signature that can be employed for predicting outcome for subjects that have pancreatic cancer. In some embodiments, the presently disclosed subject matter relates to a six gene signature that is predictive of outcome for subjects that have pancreatic ductal adenocarcinoma, and methods for using the same.

### BACKGROUND

Pancreatic ductal adenocarcinoma (PDAC), comprising over 90% of all pancreatic cancers, remains a lethal disease with an estimated 232,000 new cases and an estimated 227,000 deaths per year worldwide in 2008 (Parkin et al., 2002; Boyle & Levin, 2008). Incremental improvements in the treatment of this cancer have been made in the last two decades, but the estimated five-year survival worldwide remains at less than 5% (Boyle & Levin, 2008).

Currently, the standard of care for the 20% of patients who are diagnosed with localized disease is surgery followed by chemotherapy with gemcitabine. Unfortunately, despite the use of adjuvant therapy, median survival remains at less than two years (Neuhaus et al., 2008), with only 12% of patients undergoing curative surgery surviving more than five years (Conlon et al., 1996; Ahmad etal., 2001; Cleary et al., 2004; Han et al., 2006; Winter at al., 2006; Ferrone et al., 2008; Schnelldorfer et al., 2008).

Interestingly, in large retrospective studies examining actual long-term (five- and ten-year) survivors (Conlon etal., 1996; Ahmad etal., 2001; Cleary et al., 2004; Han at al., 2006; Winter et al., 2006; Ferrone et al., 2008; Schnelldorfer et al., 2008), only two studies (Ahmad et al., 2001; Winter et al., 2006) have found that adjuvant therapy was associated with improved survival, suggesting that the benefits of adjuvant therapy are still controversial. One possible conclusion from these studies is that tumor biology dictates outcome and that current adjuvant therapies have minimal impact on modifying this biology.

As such, defining a prognostic gene signature for pancreatic cancer would be beneficial for identifying subsets of patients that would be most or least likely to benefit from undergoing chemotherapy, by allowing future therapies to be appropriately tailored, and by providing insight into the biology that underlies the disease of long-term survivor pancreatic cancer survivors. Additionally, a prognostic signature might also facilitate defining subsets of patients that would not benefit from extirpation of their primary tumor, thus saving them from unnecessary surgery with its attendant high morbidities.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides methods for generating prognostic signatures for subjects with pancreatic ductal adenocarcinoma (PDAC). In some embodiments, the methods comprise determining expression levels for one or more genes selected from the group consisting of Fos B, KLF6, NFKBIZ, ATP4A, GSG1, and SIGLEC11 in PDAC cells obtained from a subject, wherein the determining provides a prognostic signature for the subject. In some embodiments, the methods comprise determining expression levels for at least four, five, or all six of the genes in PDAC cells obtained from the subject. In some embodiments, the methods comprise determining expression levels for each of Fos B, KLF6, NFKB/Z, ATP4A, GSG1, and SIGLEC11 in PDAC cells obtained from the subject.

In some embodiments, the methods further comprise comparing the prognostic signature determined to a standard. In some embodiments, the standard comprises a gene expression profile of the one or more genes obtained from primary PDAC cells obtained from a plurality of subjects with primary PDAC, an expression profile of the one or more genes obtained from metastatic PDAC cells obtained from a plurality of subjects with metastatic PDAC, or both. In some embodiments, the comparing comprises employing a Single Sample Predictor (SSP). In some embodiments, the gene expression profile of the one or more genes obtained from primary PDAC cells in the standard comprises a mean expression level for the one or more genes in the primary PDAC cells, the expression profile of the one or more genes obtained from metastatic PDAC cells, or both, and further wherein if the standard comprises both gene expression profiles, the mean expression levels are determined separately for the one or more genes in the primary PDAC cells and the one or more genes in the metastatic PDAC cells. In some embodiments, the standard comprises both gene expression profiles and the method further comprises assigning with the SSP the prognostic signature to either the mean expression level for the one or more genes in the primary PDAC cells or the mean expression level for the one or more genes in the metastatic PDAC cells. In some embodiments, the assigning comprises employing a Spearman correlation. In some embodiments, the assigning step, the comparing step, or both are performed on a suitably-programmed computer. In some embodiments, the subject is a human.

The presently disclosed subject matter also provides in some embodiments methods for assessing risk of an adverse outcome of a subject with pancreatic ductal adenocarcinoma (PDAC). in some embodiments, the methods comprise (a) determining a mean expression level for one or more genes selected from the group consisting of Fos B, KLF6, NFKB/Z, ATP4A, GSG1, and SIGLEC11 in a biological sample comprising PDAC cells obtained from subject; and (b) comparing the expression levels determined to a standard. In some embodiments, the subject is a human. In some embodiments, evidence of the expression level is obtained by a method comprising gene expression profiling. In some embodiments, the gene expression profiling method is a PCR-based method, a microarray based method, or an antibody-based method. In some embodiments, the expression levels are normalized relative to the expression levels of one or more reference genes, optionally by employing Lowess normalization. In some embodiments, the methods comprise determining the expression levels of at least four, five, or all six of Fos B, KLF6, NFKBIZ, ATP4A, GSG1, and SIGLEC11.

The presently disclosed subject matter also provides in some embodiments methods for predicting a clinical outcome of a treatment in a subject diagnosed with pancreatic ductal adenocarcinoma (PDAC). In some embodiments, the methods comprise (a) determining the expression level of one or more genes selected from the group consisting of Fos B, KLF6, NFKBIZ, ATP4A, GSG1, and SIGLEC11 in a biological sample comprising PDAC cells obtained from the PDAC of the subject; and (b) comparing the expression levels determined to a standard, wherein the comparing is predictive of the clinical outcome of the treatment in the subject. In some embodiments, the clinical outcome is expressed in terms of Recurrence-Free Interval (RFI), Overall Survival (OS), Disease-Free Survival (DFS), or Distant Recurrence-Free Interval (DRFI). In some embodiments, the methods comprise determining the expression levels of at least four, five, or all six of the one or more genes. In some embodiments, the treatment is selected from among surgical resection of the PDAC, chemotherapy, molecular targeted therapy, immunotherapy, and combinations thereof. In some embodiments, the standard comprises a gene expression profile of the one or more genes obtained from primary PDAC cells obtained from a plurality of subjects with primary PDAC, an expression profile of the one or more genes obtained from metastatic PDAC cells obtained from a plurality of subjects with metastatic PDAC, or both. In some embodiments, the comparing comprises employing a Single Sample Predictor (SSP). In some embodiments, the gene expression profile of the one or more genes obtained from primary PDAC cells in the standard comprises a mean expression level for the one or more genes in the primary PDAC cells, the expression profile of the one or more genes obtained from metastatic PDAC cells, or both, and further wherein if the standard comprises both gene expression profiles, the mean expression levels are determined separately for the one or more genes in the primary PDAC cells and the one or more genes in the metastatic PDAC cells. In some embodiments, the standard comprises both gene expression profiles, and the method further comprises assigning with the SSP the prognostic signature to either the mean expression level for the one or more genes in the primary PDAC cells or the mean expression level for the one or more genes in the metastatic PDAC cells. In some embodiments, the assigning comprises employing a Spearman correlation. In some embodiments, the assigning step, the comparing step, or both are performed on a suitably-programmed computer. In some embodiments, the subject is a human.

The presently disclosed subject matter also provides methods for predicting a positive or a negative clinical response of a subject with pancreatic ductal adenocarcinoma (PDAC) to a treatment. In some embodiments, the methods comprise (a) determining the expression levels of at least five genes selected from the group consisting of Fos B, KLF6, NFKB/Z, ATP4A, GSG1, and SIGLEC11 in a biological sample comprising PDAC cells obtained from the PDAC of the subject; (b) comparing the expression levels determined to a first expression profile and a second expression profile, wherein (i) the first expression profile is generated by determining the expression levels of the same at least five genes in PDAC cells obtained from a plurality of subjects with primary PDAC; (ii) the second expression profile is generated by determining the expression levels of the same at least five genes in PDAC cells obtained from a plurality of subjects with metastatic PDAC; and (iii) assigning the expression levels determined for the at least five genes in the biological sample obtained from the subject to either the first expression profile or the second expression profile, and further wherein assigning the expression levels determined for the at least five genes in the biological sample obtained from the subject to the first expression profile is indicative of a positive clinical response and assigning the expression levels determined for the at least five genes in the biological sample obtained from the subject to the second expression profile is indicative of a negative clinical response. In some embodiments, the subject is a human. In some embodiments, the expression levels of at least five genes determined are normalized as are the expression levels that make up the first and second expression profiles. In some embodiments, at least one of the first and second expression profiles was generated with Distance Weighted Discrimination (DWD). In some embodiments, one or more of the determining step, the comparing step, and the assigning step are performed on a suitably-programmed computer. In some embodiments, the treatment comprises administering gemcitabine to the subject.

The presently disclosed subject matter also provides in some embodiments arrays comprising polynucleotides hybridizing to at least five genes selected from among Fos B, KLF6, NFKBIZ, ATP4A, GSG1, and SIGLEC11 or comprising specific peptide or polypeptide gene products of at least five of Fos B, KLF6, NFKBIZ, ATP4A, GSG1, and SIGLEC11. In some embodiments, each specific peptide or polypeptide gene product present on the array is present thereon in an amount relative to each other specific peptide or polypeptide gene product that is present on the array that is reflective of the expression level of its corresponding gene in pancreatic ductal adenocarcinoma (PDAC) cells obtained from a subject with PDAC. In some embodiments, the specific peptide or polypeptide gene products are present on the array such that the array can be interrogated with at least one antibody that specifically binds to one of the specific peptide or polypeptide gene products. In some embodiments, the array comprises at least one specific peptide or polypeptide gene product for each of Fos B, KLF6, NFKBIZ, ATP4A, GSG1, and SIGLEC11.

It is thus an object of the presently disclosed subject matter to provide methods for predicting outcomes of subjects with pancreatic cancer.

An object of the presently disclosed subject matter having been stated hereinabove, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A-1E are a series of heat maps and plots that relate to the identification, development, and application of a six-gene signature for pancreatic ductal adenocarcinoma (PDAC).

FIG. 1A is a gene expression heat map showing clustering of the six genes defined by Significance Analysis of Microarrays (SAM) evaluation of the metastatic compared to non-metastatic primary PDAC using a false discovery rate of 5%. FIG. 1B is a gene expression heat map of patient samples divided into high- and low-risk groups in a training set of 34 patients with localized and resected PDAC using the X-tile determined cut-point of a Pearson correlation coefficient of zero. FIG. 1C is a gene expression heat map of patient samples divided into high- and low-risk groups in an independent test set of 67 patients with localized and resected PDAC using the predetermined cut-point of zero.

FIG. 1D is a Kaplan-Meier survival curve of the training set classified into high- and low-risk groups according to the X-tile determined cut-point of a Pearson correlation coefficient of zero. FIG. 1E is a Kaplan-Meier survival curve of the independent test set classified into high- and low-risk groups according to the same predetermined cut-point.

FIGS. 2A-2C depict the results of experiments investigating the significances of KLF6 and Fos B expression in primary PDAC.

FIG. 2A is a bar graph showing that KLF6 expression is significantly higher in PDAC compared to normal adjacent pancreas in an independent dataset of a 50-patient tissue microarray (TMA; UNC2) as well as University of Nebraska Medical Center Rapid Autopsy Pancreatic Program (NEB) samples used for the original analysis. FIG. 2B is a Kaplan-Meier survival curve of 50 patients classified by high and low KLF6 scores, using a median cutoff score of 1.5 (see discussion in EXAMPLE 5). FIG. 2C is a series of photomicrographs depicting KLF6 immunostaining in the primary tumor of a patient who died of metastatic disease (Panel ii) and in a resected primary tumor (Panel iv). Minimal staining is seen in the matched normal adjacent tissue of both patients (Panels i and iii, respectively). KLF6 immunostaining in islet cells is indicated with a white arrowhead in Panel 2C(i). Arrows indicate normal ductal epithelium. Black arrowheads indicate tumor sites.

### BRIEF DESCRIPTION OF THE SEQUENCE LISTING

SEQ ID NOs: 1-28 as summarized in Table 1 are nucleotide and amino acid sequences of various human gene products the expression of which can be employed with respect to the presently disclosed methods and arrays.

TABLE 1
Listing of GENBANK ® Accession Numbers for Nucleic Acid
and Amino Acid Sequences of Exemplary Gene Products

| | GENBANK Accession No. | |
|---|---|---|
| Description | Nucleic Acid | Amino Acid |
| Human Fos B transcript | NM_006732; | NP_006723; |
| variant 1 | SEQ ID NO: 1 | SEQ ID NO: 2 |
| Human Fos B transcript | NM_001114171; | NP_001107643; |
| variant 2 | SEQ ID NO: 3 | SEQ ID NO: 4 |
| Human KLF6 transcript | NM_001300; | NP_001291; |
| variant A | SEQ ID NO: 5 | SEQ ID NO: 6 |
| Human KLF6 transcript | NM_001160124; | NP_001153596; |
| variant B | SEQ ID NO: 7 | SEQ ID NO: 8 |
| Human KLF6 transcript | NM_001160125; | NP_001153597.1; |
| variant C | SEQ ID NO: 9 | SEQ ID NO: 10 |
| Human NFKBIZ transcript | NM_031419; | NP_113607; |
| variant 1 | SEQ ID NO: 11 | SEQ ID NO: 12 |
| Human NFKBIZ transcript | NM_001005474; | NP_001005474; |
| variant 2 | SEQ ID NO: 13 | SEQ ID NO: 14 |
| Human ATP4A | NM_000704; | NP_000695; |
| | SEQ ID NO: 15 | SEQ ID NO: 16 |
| Human GSG1 transcript | NM_031289; | NP_112579; |
| variant 1 | SEQ ID NO: 17 | SEQ ID NO: 18 |
| Human GSG1 transcript | NM_153823; | NP_722545; |
| variant 2 | SEQ ID NO: 19 | SEQ ID NO: 20 |
| Human GSG1 transcript | NM_001080554; | NP_001074023; |
| variant 3 | SEQ ID NO: 21 | SEQ ID NO: 22 |
| Human GSG1 transcript | NM_001080555; | NP_001074024; |
| variant 4 | SEQ ID NO: 23 | SEQ ID NO: 24 |
| Human SIGLEC11 | NM_052884; | NP_443116; |
| transcript variant 1 | SEQ ID NO: 25 | SEQ ID NO: 26 |
| Human SIGLEC11 | NM_001135163; | NP_001128635; |
| transcript variant 2 | SEQ ID NO: 27 | SEQ ID NO: 28 |

All of the sequences listed in Table 1, including all annotations and references cited in the corresponding GENBANK® entries, are incorporated herein by reference in their entireties.

### DETAILED DESCRIPTION

The present subject matter will be now be described more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### I. General Considerations

To date, expression profiling of pancreatic cancers has led to occasional information regarding gene expression changes with respect to molecular diagnostic and prognostic markers (Grutzmann et al., 2004; Grutzmann et al., 2005; Goggins, 2007; Grote & Logsdon, 2007; Tonini et al., 2007; Kolbert et al., 2008). However, the search for genes that are of biological significance in these large datasets continues to present significant challenges.

Disclosed herein are comparisons of primary PDAC tumors at the extremes of disease, wherein molecular changes reflective of differences in biology within primary PDAC tumors have been identified. The data presented herein show that there are distinct molecular changes in patients with primary PDAC, and that these alterations can be exploited for the study of novel targets. The prognostic value of these gene expression differences has also been evaluated, and the presently disclosed subject matter shows that they retain their prognostic value in multiple independent datasets. The prognostic signature can therefore be used to define patients most likely to benefit from surgery or chemotherapy and/or to stratify patients in future clinical trials.

### II. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, the phrase “a cell” refers to one or more cells, unless the context clearly indicates otherwise.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including”, “containing”, and “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting or excludes any element, step, and/or ingredient not specifically recited. For example, when the phrase “consists of appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, the presently disclosed subject matter in some embodiments can “consist essentially of determining expression levels for one or more genes selected from among Fos B, KLF6, NFKBIZ, ATP4A, GSG1, and SIGLEC11 in PDAC cells obtained from a subject, which means that the recited gene(s) is/are the only genes for which an expression level or expression levels are determined. It is noted, however, that expression levels for various positive and/or negative control genes can also be determined, for example, to standardize and/or normalize the expression levels of Fos B, KLF6, NFKBIZ, ATP4A, GSG1, and SIGLEC11 in PDAC cells (if desired).

With respect to the terms “comprising”, “consisting essentially of”, and “consisting of”, where one of these three terms is used herein, the presently disclosed and claimed subject matter can include the use of either of the other two terms. For example, the presently disclosed subject matter relates in some embodiments to arrays comprising polynucleotides hybridizing to at least five genes selected from among Fos B, KLF6, NFKBIZ, ATP4A, GSG1, and SIGLEC11 and/or comprising specific peptide or polypeptide gene products of at least five of Fos B, KLF6, NFKBIZ, ATP4A, GSG1, and SIGLEC11. It is understood that the presently disclosed subject matter thus also encompasses arrays that in some embodiments consist essentially of polynucleotides hybridizing to at least five genes selected from among Fos B, KLF6, NFKBIZ, ATP4A, GSG1, and SIGLEC11 and/or consisting essentially of specific peptide or polypeptide gene products of at least five of Fos B, KLF6, NFKBIZ, ATP4A, GSG1, and SIGLEC11, as well as arrays that in some embodiments consist of polynucleotides hybridizing to at least five genes selected from among Fos B, KLF6, NFKBIZ, ATP4A, GSG1, and SIGLEC11 and/or consist of specific peptide or polypeptide gene products of at least five of Fos B, KLF6, NFKBIZ, ATP4A, GSG1, and SIGLEC11. Similarly, it is also understood that in some embodiments the methods of the presently disclosed subject matter comprise the steps that are disclosed herein and/or that are recited in the claims, in some embodiments the methods of the presently disclosed subject matter consist essentially of the steps that are disclosed herein and/or that are recited in the claims, and in some embodiments the methods of the presently disclosed subject matter consist of the steps that are disclosed herein and/or that are recited in the claim.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein. In some embodiments, the presently disclosed subject matter relates to human subjects.

Similarly, all genes, gene names, and gene products disclosed herein are intended to correspond to orthologs from any species for which the compositions and methods disclosed herein are applicable. Thus, the terms include, but are not limited to genes and gene products from humans. It is understood that when a gene or gene product from a particular species is disclosed, this disclosure is intended to be exemplary only, and is not to be interpreted as a limitation unless the context in which it appears clearly indicates. Thus, for example, the genes and/or gene products disclosed herein are also intended to encompass homologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals and birds. More particularly provided is the use of the methods and compositions of the presently disclosed subject matter on mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the application of the methods and compositions of the presently disclosed subject matter to livestock, including but not limited to domesticated swine (pigs and hogs), ruminants, horses, poultry, and the like.

The term “about”, as used herein when referring to a measurable value such as an amount of weight, time, dose, etc., is meant to encompass variations of in some embodiments ±20%, in some embodiments ±10%, in some embodiments ±5%, in some embodiments ±1%, and in some embodiments ±0.1% from the specified amount, as such variations are appropriate to perform the disclosed methods and/or to employ the presently disclosed arrays.

As used herein the term “gene” refers to a hereditary unit including a sequence of DNA that occupies a specific location on a chromosome and that contains the genetic instruction for a particular characteristic or trait in an organism. Similarly, the phrase “gene product” refers to biological molecules that are the transcription and/or translation products of genes. Exemplary gene products include, but are not limited to mRNAs and polypeptides that result from translation of mRNAs. Any of these naturally occurring gene products can also be manipulated in vivo or in vitro using well known techniques, and the manipulated derivatives can also be gene products. For example, a cDNA is an enzymatically produced derivative of an RNA molecule (e.g., an mRNA), and a cDNA is considered a gene product. Additionally, polypeptide translation products of mRNAs can be enzymatically fragmented using techniques well known to those of skill in the art, and these peptide fragments are also considered gene products.

As used herein, the term “Fos B” refers to the FBJ murine osteosarcoma viral oncogene homolog B (Fos B) gene. Exemplary, non-limiting Fos B gene products from humans are described in GENBANK® Accession Nos. NM_{—}006732, NM_{—}001114171, NP_{—}006723, and NP_{—}001107643.

As used herein, the term “KLF6” refers to the Kruppel-like factor 6 gene. Exemplary, non-limiting KLF6 gene products from humans are described in GENBANK® Accession Nos. NM_{—}001300, NM_{—}001160124, NM_{—}001160125, NP_{—}001291, NP_{—}001153596, and NP_{—}001153597.1.

As used herein, the term “NFKBIZ' refers to the nuclear factor of kappa light polypeptide gene enhancer in B-cells inhibitor, zeta gene. Exemplary, non-limiting NFKBIZ gene products are described in GENBANK® Accession Nos. NM_{—}031419, NM_{—}001005474, NP_{—}113607, and NP_{—}001005474.

As used herein, the term “ATP4A” refers to the ATPase, H+/K+ exchanging, alpha polypeptide gene. Exemplary, non-limiting ATP4A gene products are described in GENBANK® Accession Nos. NM_{—}000704 and NP_{—}000695.

As used herein, the term “GSG1” refers to the germ cell associated 1 gene. Exemplary, non-limiting GSG1 gene products are described in GENBANK® Accession Nos. NM_{—}031289, NM_{—}153823, NM_{—}001080554, NM_{—}001080555, NP_{—}112579, NP_{—}722545, NP_{—}001074023, and NP_{—}001074024.

As used herein, the term “SIGLEC11” refers to the sialic acid binding Ig-like lectin 11 gene. Exemplary, non-limiting SIGLEC11 gene products are described in GENBANK® Accession Nos. NM_{—}052884, NM_{—}001135163, NP_{—}443116, and NP_{—}001128635.

It is understood that while the nucleotide and amino acid sequences for the human orthologs of Fos B, KLF6, NFKBIZ, ATP4A, GSG1, and SIGLEC11 are disclosed herein, orthologs of these genes from other species are also included within the presently disclosed subject matter.

The term “isolated”, as used in the context of a nucleic acid or polypeptide (including, for example, a nucleotide sequence, a polypeptide, and/or a peptide), indicates that the nucleic acid or polypeptide exists apart from its native environment. An isolated nucleic acid or polypeptide can exist in a purified form or can exist in a non-native environment.

Further, as used for example in the context of a cell, nucleic acid, polypeptide, or peptide, the term “isolated” indicates that the cell, nucleic acid, polypeptide, or peptide exists apart from its native environment. In some embodiments, “isolated” refers to a physical isolation, meaning that the cell, nucleic acid, polypeptide, or peptide has been removed from its native environment (e.g., from a subject).

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, and “mRNA”. Nucleic acids can be synthesized, or can be derived from any biological source, including any organism.

As used herein, the terms “peptide” and “polypeptide” refer to polymers of at least two amino acids linked by peptide bonds. Typically, “peptides” are shorter than “polypeptides”, but unless the context specifically requires, these terms are used interchangeably herein.

As used herein, a cell, nucleic acid, or peptide exists in a “purified form” when it has been isolated away from some, most, or all components that are present in its native environment, but also when the proportion of that cell, nucleic acid, or peptide in a preparation is greater than would be found in its native environment. As such, “purified” can refer to cells, nucleic acids, and peptides that are free of all components with which they are naturally found in a subject, or are free from just a proportion thereof.

### III. Methods for Generating Prognostic Signatures

In some embodiments, the presently disclosed subject matter provides methods for generating prognostic signatures for a subject with cancer (e.g., pancreatic ductal adenocarcinoma (PDAC)). As used herein, the phrase “prognostic signature” refers to a gene expression profile comprising gene expression levels for one, two, three, four, five, or six of the genes disclosed herein (e.g., Fos B, KLF6, NFKBIZ, ATP4A, GSG1, and SIGLEC11) in PDAC cells obtained from the subject, wherein the determining provides a prognostic signature for the subject. In some embodiments, a gene expression profile of the presently disclosed subject matter can comprise gene expression levels for KLF6 in combination with any or all of Fos B, NFKBIZ, ATP4A, GSG1, and SIGLEC11, as well as all subcombinations thereof. By way of example and not limitation, the presently disclosed methods employ determinations of gene expression levels (e.g., absolute gene expression levels and/or relative gene expression levels, wherein the relative gene expression levels are calculated with respect to a standard) of any or all of the following combinations and subcombinations of genes: KLF6 alone; KLF6 and Fos B; KLF6 and NFKBIZ; KLF6 and ATP4A; KLF6 and GSG1; KLF6 and SIGLEC11; KLF6, Fos B, and NFKBIZ; KLF6, Fos B, and ATP4A; KLF6, Fos B, and GSG1, KLF6, Fos B, and SIGLEC11; KLF6, NFKBIZ, and ATP4A; KLF6, NFKBIZ, and GSG1; KLF6, NFKBIZ, and SIGLEC11; KLF6, ATP4A, and GSG1; KLF6, ATP4A, and SIGLEC11; KLF6, GSG1, and SIGLEC11; KLF6, Fos B, NFKBIZ, and ATP4A; KLF6, Fos B, NFKBIZ, and GSG1; KLF6, Fos B, NFKBIZ, and SIGLEC11; KLF6, NFKBIZ, ATP4A, and GSG1; KLF6, NFKBIZ, ATP4A, and SIGLEC11; KLF6, ATP4A, GSG1, and SIGLEC11; KLF6, Fos B, NFKBIZ, ATP4A, and GSG1; KLF6, Fos B, NFKBIZ, ATP4A, and SIGLEC11; and/or KLF6, NFKBIZ, ATP4A, GSG1, and SIGLEC11. In some embodiments, expression levels for each of Fos B, KLF6, NFKBIZ, ATP4A, GSG1, and SIGLEC11 are determined.

As disclosed herein, such gene expression profiles can be predictive of various clinical outcomes, for example, by comparing to appropriate standards.

In some embodiments, methods for generating prognostic signatures further comprise comparing the derived prognostic signatures to one or more standards. As used herein, the term “standard” refers to an entity to which another entity (e.g., a prognostic signature) can be compared such that the comparison provides information of interest. An exemplary standard that is described herein is a test set. Additional discussion of standards can be found herein below. Such a comparison can be carried out on an apparatus, such as a system comprising a suitably programmed computer.

Thus, a profile can be created once an expression level is determined for a gene. As used herein, the term “profile” (e.g., a “gene expression profile”) refers to a repository of the expression level data that can be used to compare the expression levels of one or more genes, such as but not limited to one or more different genes among various subjects. For example, for a given subject, the term “profile” can encompass the expression levels of one or more of the genes disclosed herein detected in whatever units are chosen.

The term “profile” is also intended to encompass manipulations of the expression level data derived from a subject. For example, once relative expression levels are determined for a given set of genes in a subject, the relative expression levels for that subject can be compared to a standard to determine if the expression levels in that subject are higher or lower than for the same genes in the standard. Standards can include any data deemed to be relevant for comparison. Such a comparison can be carried out on an apparatus, such as a system comprising a suitably programmed computer.

### IV. Methods for Assessing Risks of Adverse Outcomes

The presently disclosed subject matter also provides methods for assessing risk of an adverse outcome of a subject with pancreatic ductal adenocarcinoma (PDAC).

In some embodiments, the methods comprise determining an expression level for one or more genes selected from among Fos B, KLF6, NFKBIZ, ATP4A, GSG1, and SIGLEC11 in a biological sample comprising PDAC cells obtained from subject; and comparing the expression levels determined to a standard. By way of example and not limitation, the presently disclosed methods employ determinations of gene expression levels (e.g., absolute gene expression levels and/or relative gene expression levels, wherein the relative gene expression levels are calculated with respect to a standard) of any or all of the following combinations and subcombinations of genes: KLF6 alone; KLF6 and Fos B; KLF6 and NFKBIZ; KLF6 and ATP4A; KLF6 and GSG1; KLF6 and SIGLEC11; KLF6, Fos B, and NFKBIZ; KLF6, Fos B, and ATP4A; KLF6, Fos B, and GSG1; KLF6, Fos B, and SIGLEC11; KLF6, NFKBIZ, and ATP4A; KLF6, NFKBIZ, and GSG1; KLF6, NFKBIZ, and SIGLEC11; KLF6, ATP4A, and GSG1; KLF6, ATP4A, and SIGLEC11; KLF6, GSG1, and SIGLEC11; KLF6, Fos B, NFKBIZ, and ATP4A; KLF6, Fos B, NFKBIZ, and GSG1; KLF6, Fos B, NFKBIZ, and SIGLEC11; KLF6, NFKBIZ, ATP4A, and GSG1; KLF6, NFKBIZ, ATP4A, and SIGLEC11; KLF6, ATP4A, GSG1, and SIGLEC11; KLF6, Fos B, NFKBIZ, ATP4A, and GSG1; KLF6, Fos B, NFKBIZ, ATP4A, and SIGLEC11; and/or KLF6, NFKBIZ, ATP4A, GSG1, and SIGLEC11. In some embodiments, expression levels for each of Fos B, KLF6, NFKBIZ, ATP4A, GSG1, and SIGLEC11 are determined.

In some embodiments, the comparing step is indicative of an increased likelihood that an adverse outcome (including, but not limited to decreased Overall Survival (OS) and/or Disease-Free Survival (DFS)) would occur in a subject relative to other subjects with PDAC. Such a comparison can be carried out on an apparatus, such as a system comprising a suitably programmed computer.

V. Methods for Predicting Clinical Outcomes from Treatments

The presently disclosed subject matter also provides methods for predicting a clinical outcome of a treatment in a subject diagnosed with pancreatic ductal adenocarcinoma (PDAC). In some embodiments, the methods comprise (a) determining the expression level of one or more genes selected from among Fos B, KLF6, NFKBIZ, ATP4A, GSG1, and SIGLEC11 in a biological sample comprising PDAC cells obtained from the PDAC of the subject; and (b) comparing the expression levels determined to a standard, wherein the comparing is predictive of the clinical outcome of the treatment in the subject.

As used herein, the phrase “clinical outcome” refers to any measure by which a treatment designed to treat PDAC can be measured. Exemplary clinical outcomes include Recurrence-Free Interval (RFI), Overall Survival (OS), Disease-Free Survival (DFS), or Distant Recurrence-Free Interval (DRFI). In some embodiments, the comparison can be carried out on an apparatus, such as a system comprising a suitably programmed computer.

By way of example and not limitation, the presently disclosed methods employ determinations of gene expression levels (e.g., absolute gene expression levels and/or relative gene expression levels, wherein the relative gene expression levels are calculated with respect to a standard) of any or all of the following combinations and subcombinations of genes: KLF6 alone; KLF6 and Fos B; KLF6 and NFKBIZ; KLF6 and ATP4A; KLF6 and GSG1; KLF6 and SIGLEC11; KLF6, Fos B, and NFKBIZ; KLF6, Fos B, and ATP4A; KLF6, Fos B, and GSG1; KLF6, Fos B, and SIGLEC11; KLF6, NFKBIZ, and ATP4A; KLF6, NFKBIZ, and GSG1; KLF6, NFKBIZ, and SIGLEC11; KLF6, ATP4A, and GSG1; KLF6, ATP4A, and SIGLEC11; KLF6, GSG1, and SIGLEC11; KLF6, Fos B, NFKBIZ, and ATP4A; KLF6, Fos B, NFKBIZ, and GSG1; KLF6, Fos B, NFKBIZ, and SIGLEC11; KLF6, NFKBIZ, ATP4A, and GSG1; KLF6, NFKBIZ, ATP4A, and SIGLEC11; KLF6, ATP4A, GSG1, and SIGLEC11; KLF6, Fos B, NFKBIZ, ATP4A, and GSG1; KLF6, Fos B, NFKBIZ, ATP4A, and SIGLEC11; and/or KLF6, NFKBIZ, ATP4A, GSG1, and SIGLEC11. In some embodiments, expression levels for each of Fos B, KLF6, NFKBIZ, ATP4A, GSG1, and SIGLEC11 are determined.

### VI. Methods for Predicting a Positive or a Negative Clinical Response in a Subject

The presently disclosed subject matter also provides methods for predicting a positive or a negative clinical response of a subject with pancreatic ductal adenocarcinoma (PDAC) to a treatment such as, but not limited to treatment with gemcitabine. In some embodiments, the methods comprise (a) determining the expression levels of at least one, two, three, four, or five genes selected from among Fos B, KLF6, NFKBIZ, ATP4A, GSG1, and SIGLEC11 in a biological sample comprising PDAC cells obtained from the PDAC of the subject; and (b) comparing the expression, levels determined to a first expression profile and a second expression profile, wherein (i) the first expression profile is generated by determining the expression levels of the same at least one, two, three, four, or five genes in PDAC cells obtained from a plurality of subjects with primary PDAC; (ii) the second expression profile is generated by determining the expression levels of the same at least one, two, three, four, or five genes in PDAC cells obtained from a plurality of subjects with metastatic PDAC; and (iii) the comparing assigns the expression levels determined for the at least one, two, three, four, or five genes in the biological sample obtained from the subject to either the first expression profile or the second expression profile, and further wherein assigning the expression levels determined for the at least one, two, three, four, or five genes in the biological sample obtained from the subject to the first expression profile is indicative of a positive clinical response and assigning the expression levels determined for the at least one, two, three, four, or five genes in the biological sample obtained from the subject to the second expression profile is indicative of a negative clinical response. In some embodiments, the first, the second, or both the first and second expression levels are mean expression levels. In some embodiments, the comprising comprises employing a Single Sample Predictor (SSP).

By way of example and not limitation, the presently disclosed methods employ determinations of gene expression levels (e.g., absolute gene expression levels and/or relative gene expression levels, wherein the relative gene expression levels are calculated with respect to a standard) of any or all of the following combinations and subcombinations of genes: KLF6 alone; KLF6 and Fos B; KLF6 and NFKBIZ; KLF6 and ATP4A; KLF6 and GSG1;

KLF6 and SIGLEC11; KLF6, Fos B, and NFKBIZ; KLF6, Fos B, and ATP4A; KLF6, Fos B, and GSG1; KLF6, Fos B, and SIGLEC11; KLF6, NFKBIZ, and ATP4A; KLF6, NFKBIZ, and GSG1; KLF6, NFKBIZ, and SIGLEC11; KLF6, ATP4A, and GSG1; KLF6, ATP4A, and SIGLEC11; KLF6, GSG1, and SIGLEC11; KLF6, Fos B, NFKBIZ, and ATP4A; KLF6, Fos B, NFKBIZ, and GSG1; KLF6, Fos B, NFKBIZ, and SIGLEC11; KLF6, NFKBIZ, ATP4A, and GSG1; KLF6, NFKBIZ, ATP4A, and SIGLEC11; KLF6, ATP4A, GSG1, and SIGLEC11; KLF6, Fos B, NFKBIZ, ATP4A, and GSG1; KLF6, Fos B, NFKBIZ, ATP4A, and SIGLEC11; and/or KLF6, NFKBIZ, ATP4A, GSG1, and SIGLEC11. In some embodiments, expression levels for each of Fos B, KLF6, NFKBIZ, ATP4A, GSG1, and SIGLEC11 are determined. In some embodiments, the comparison can be carried out on an apparatus, such as a system comprising a suitably programmed computer.

### VII. Methods of Gene Expression Analysis

VII.A. Assay Formats

The genes identified as being differentially expressed in, for example, primary PDAC versus metastatic PDAC, can be used in a variety of nucleic acid detection assays to detect or quantitate the expression level of a gene or multiple genes in a given sample. For example, Northern blotting, nuclease protection, RT-PCR (e.g., quantitative RT-PCR; QRT-PCR), and/or differential display methods can be used for detecting gene expression levels. In some embodiments, methods and assays of the presently disclosed subject matter are employed with array or chip hybridization-based methods and systems for detecting the expression of a plurality of genes.

Any hybridization assay format can be used, including solution-based and solid support-based assay formats. Representative solid supports containing oligonucleotide probes for differentially expressed genes of the presently disclosed subject matter can be filters, polyvinyl chloride dishes, silicon, glass based chips, etc. Such wafers and hybridization methods are widely available and include, for example, those disclosed in PCT International Patent Application Publication WO 95/11755). Any solid surface to which oligonucleotides can be bound, either directly or indirectly, either covalently or non-covalently, can be used. An exemplary solid support is a high-density array or DNA chip. These contain a particular oligonucleotide probe in a predetermined location on the array. Each predetermined location can contain more than one molecule of the probe, but in some embodiments each molecule within the predetermined location has an identical sequence. Such predetermined locations are termed features. There can be any number of features on a single solid support including, for example, about 2, 10, 100, 1000, 10,000, 100,000, or 400,000 of such features on a single solid support. The solid support, or the area within which the probes are attached, can be of any convenient size (for example, on the order of a square centimeter).

Oligonucleotide probe arrays for differential gene expression monitoring can be made and employed according to any techniques known in the art (see e.g., Lockhart et al., 1996; McCall et al., 1996). Such probe arrays can contain at least two or more oligonucleotides that are complementary to or hybridize to two or more of the genes described herein. Such arrays can also contain oligonucleotides that are complementary or hybridize to at least about 2, 3, 4, 5, 6, 7, 8, 9, 10, 15, 20, 25, 30, 50, 70, 100, or more of the nucieic acid sequences disclosed herein.

The genes that are assayed according to the presently disclosed subject matter are typically in the form of RNA (e.g., total RNA or mRNA) and/or reverse transcribed RNA (i.e., cDNA), including subsequences thereof. The genes can be cloned or not, and the genes can be amplified or not. In some embodiments, poly A⁺ RNA is employed as a source.

Probes based on the sequences of the genes described herein can be prepared by any commonly available method. Oligonucleotide probes for assaying the tissue or cell sample are in some embodiments of sufficient length to specifically hybridize only to appropriate complementary genes or transcripts. Typically, the oligonucleotide probes are at least 10, 12, 14, 16, 18, 20, or 25 nucleotides in length. In some embodiments, longer probes of at least 30, 40, 50, or 60 nucleotides are employed.

As used herein, oligonucleotide sequences that are complementary to one or more of the genes described herein are oligonucleotides that are capable of hybridizing under stringent conditions to at least part of the nucleotide sequence of said genes. Such hybridizable oligonucleotides will typically exhibit in some embodiments at least about 75% sequence identity, in some embodiments about 80% sequence identity, in some embodiments about 85% sequence identity, in some embodiments about 90% sequence identity, in some embodiments about 91% sequence identity, in some embodiments about 92% sequence identity, in some embodiments about 93% sequence identity, in some embodiments about 94% sequence identity, in some embodiments about 95% sequence identity, and in some embodiments greater than 95% sequence identity (e.g., 96%, 97%, 98%, 99%, or 100% sequence identity) at the nucleotide level to the nucleic acid sequences disclosed herein.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals can also be produced by intrinsic fluorescence of the array components themselves. A single background signal can be calculated for the entire array, or a different background signal can be calculated for each target nucleic acid. In some embodiments, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack probes.

Assays, methods, and systems of the presently disclosed subject matter can utilize available formats to simultaneously screen in some embodiments at least about 10, in some embodiments at least about 50, in some embodiments at least about 100, in some embodiments at least about 1000, in some embodiments at least about 10,000, and in some embodiments at least about 40,000 or more different nucleic acid hybridizations.

As used herein, a “probe” is defined as a nucleic acid that is capable of binding to a target nucleic acid of complementary sequence through one or more types of chemical bonds, usually through complementary base pairing, usually through hydrogen bond formation. As used herein, a probe can include natural (i.e., A, G, U, C, or T) or modified bases (7-deazaguanosine, inosine, etc.). In addition, the bases in probes can be joined by a linkage other than a phosphodiester bond, so long as it does not interfere with hybridization. Thus, probes can be peptide nucleic acids in which the constituent bases are joined by peptide bonds rather than phosphodiester linkages.

The terms “mismatch control” and “mismatch probe” refer to a probe comprising a sequence that is deliberately selected not to be perfectly complementary to a particular target sequence. For each mismatch (MM) control in a high-density array there typically exists a corresponding perfect match (PM) probe that is perfectly complementary to the same particular target sequence. The mismatch can comprise one or more bases.

While the mismatch(s) can be located anywhere in the mismatch probe, terminal mismatches are less desirable as a terminal mismatch is less likely to prevent hybridization of the target sequence. In some embodiments, the mismatch is located at or near the center of the probe such that the mismatch is most likely to destabilize the duplex with the target sequence under the test hybridization conditions.

The phrase “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe, or the like. A perfect match control or perfect match probe is, however, distinguished from a “mismatch control” or “mismatch probe”.

VII.B. Probe Design

Upon review of the present disclosure, one of skill in the art will appreciate that an enormous number of array designs are suitable for the practice of the presently disclosed subject matter. The high-density array typically includes a number of probes that specifically hybridize to the sequences of interest. See PCT International Patent Application Publication WO 99/32660, incorporated herein by reference in its entirety, for methods of producing probes for a given gene or genes. In addition, in some embodiments, the array includes one or more control probes.

High-density array chips of the presently disclosed subject matter include in some embodiments “test probes”. Test probes can be oligonucleotides that in some embodiments range from about 5 to about 500 or about 5 to about 50 nucleotides, in some embodiments from about 10 to about 40 nucleotides, and in some embodiments from about 15 to about 40 nucleotides in length. In some embodiments, the probes are about 20 to 25 nucleotides in length. In some embodiments, test probes are double or single strand DNA sequences. DNA sequences are isolated or cloned from natural sources and/or amplified from natural sources using natural nucleic acid as templates. These probes have sequences complementary to particular subsequences of the genes the expression of which they are designed to detect. Thus, the test probes are capable of specifically hybridizing to the target nucleic acid they are to detect.

In addition to test probes that bind the target nucleic acid(s) of interest, the high-density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that can cause the signal of a perfect hybridization to vary between arrays. In some embodiments, signals (e.g., fluorescence intensity) read from some or all other probes in the array are divided by the signal (e.g., fluorescence intensity) from the control probes, thereby normalizing the measurements.

Virtually any probe can serve as a normalization control. However, it is recognized that hybridization efficiency varies with base composition and probe length. Exemplary normalization probes can be selected to reflect the average length of the other probes present in the array; however, they can be selected to cover a range of lengths. The normalization control(s) can also be selected to reflect the (average) base composition of the other probes in the array; however, in some embodiments, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to, the β-actin gene, the transferrin receptor gene, the GAPDH gene, and the like. Exemplary human housekeeping genes and the corresponding GENBANK® Accession Nos. therefor are disclosed in

Mismatch controls can also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). In some embodiments, mismatch probes contain one or more central mismatches. Thus, for example, where a probe is a 20-mer, a corresponding mismatch probe will have the identical sequence except for a single base mismatch (e.g., substituting a G, a C, or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a given hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (IBM)-I(MM)) provides a good measure of the concentration of the hybridized material.

VII.C. Nucleic Acid Samples

A biological sample that can be analyzed in accordance with the presently disclosed subject matter comprises in some embodiments a nucleic acid. The terms “nucleic acid”, “nucleic acids”, and “nucleic acid molecules” each refer in some embodiments to deoxyribonucleotides, ribonucleotides, and polymers and folded structures thereof in either single- or double-stranded form. Nucleic acids can be derived from any source, including any organism. Deoxyribonucleic acids can comprise genomic DNA, cDNA derived from ribonucleic acid, DNA from an organelle (e.g., mitochondrial DNA or chloroplast DNA), or combinations thereof. Ribonucleic acids can comprise genomic RNA (e.g., viral genomic RNA), messenger RNA (mRNA), ribosomal RNA (rRNA), transfer RNA (tRNA), or combinations thereof.

VII.C.1. Isolation of Nucleic Acid Samples

Nucleic acid samples used in the methods and assays of the presently disclosed subject matter can be prepared by any available method or process. Methods of isolating total mRNA are also known to those of skill in the art. For example, methods of isolation and purification of nucleic acids are described in detail in Chapter 3 of Tijssen, 1993. Such samples include RNA samples, but also include cDNA synthesized from an mRNA sample isolated from a cell or tissue of interest. Such samples also include DNA amplified from the cDNA, an RNA transcribed from the amplified DNA, and combinations thereof. One of skill in the art would appreciate that it can be desirable to inhibit or destroy RNase present in homogenates before homogenates are used as a source of RNA.

The presently disclosed subject matter encompasses use of a sufficiently large biological sample to enable a comprehensive survey of low abundance nucleic acids in the sample. Thus, the sample can optionally be concentrated prior to isolation of nucleic acids. Several protocols for concentration have been developed that alternatively use slide supports (Kohsaka & Carson, 1994; Millar et al., 1995), filtration columns (Bej et al., 1991), or immunomagnetic beads (Albert et al., 1992; Cousins et al., 1992). Such approaches can significantly increase the sensitivity of subsequent detection methods.

As one example, SEPHADEX® matrix (Sigma of St. Louis, Mo., United States of America) is a matrix of diatomaceous earth and glass suspended in a solution of chaotropic agents and has been used to bind nucleic acid material (Boom et al., 1990; Buffone et al., 1991). After the nucleic acid is bound to the solid support material, impurities and inhibitors are removed by washing and centrifugation, and the nucleic acid is then eluted into a standard buffer. Target capture also allows the target sample to be concentrated into a minimal volume, facilitating the automation and reproducibility of subsequent analyses (Lanciotti et al., 1992).

Methods for nucleic acid isolation can comprise simultaneous isolation of total nucleic acid, or separate and/or sequential isolation of individual nucleic acid types (e.g., genomic DNA, cDNA, organelle DNA, genomic RNA, mRNA, poly A⁺RNA, rRNA, tRNA) followed by optional combination of multiple nucleic acid types into a single sample.

When RNA (e.g., mRNA) is selected for analysis, the disclosed methods allow for an assessment of gene expression in the tissue or cell type from which the RNA was isolated. RNA isolation methods are known to one of skill in the art. See Albert et al., 1992; Busch et al., 1992; Hamel et al., 1995; Herrewegh et al., 1995; lzraeli et al., 1991; McCaustland et al., 1991; Natarajan et al., 1994; Rupp et al., 1988; Tanaka et al., 1994; and Van Kerckhoven et al., 1994.

Simple and semi-automated extraction methods can also be used for nucleic acid isolation, including for example, the SPLIT SECOND™ system (Boehringer Mannheim of Indianapolis, Ind., United States of America), the TRIZOL™ Reagent system (Life Technologies of Gaithersburg, Md., United States of America), and the FASTPREP™ system (Bio 101 of La Jolla, Calif., United States of America). See also Smith 1998; and Paladichuk 1999.

In some embodiments, nucleic acids that are used for subsequent amplification and labeling are analytically pure as determined by spectrophotometric measurements or by visual inspection following electrophoretic resolution. In some embodiments, the nucleic acid sample is free of contaminants such as polysaccharides, proteins, and inhibitors of enzyme reactions. When a biological sample comprises an RNA molecule that is intended for use in producing a probe, it is preferably free of DNase and RNase. Contaminants and inhibitors can be removed or substantially reduced using resins for DNA extraction (e.g., CHELEX™ 100 from Bio-Rad Laboratories of Hercules, Calif., United States of America) or by standard phenol extraction and ethanol precipitation.

VII.C.2. Amplification of Nucleic Acid Samples

In some embodiments, a nucleic acid isolated from a biological sample is amplified prior to being used in the methods disclosed herein. In some embodiments, the nucleic acid is an RNA molecule, which is converted to a complementary DNA (cDNA) prior to amplification. Techniques for the isolation of RNA molecules and the production of cDNA molecules from the RNA molecules are known (see generally, Silhavy et al., 1984; Sambrook & Russell, 2001; Ausubel et al., 2002; and Ausubel et al., 2003). In some embodiments, the amplification of RNA molecules isolated from a biological sample is a quantitative amplification (e.g., by quantitative RT-PCR).

The terms “template nucleic acid” and “target nucleic acid” as used herein each refer to nucleic acids isolated from a biological sample as described herein above. The terms “template nucleic acid pool”, “template pool”, “target nucleic acid pool”, and “target pool” each refer to an amplified sample of “template nucleic acid”. Thus, a target pool comprises amplicons generated by performing an amplification reaction using the template nucleic acid. In some embodiments, a target pool is amplified using a random amplification procedure as described herein.

The term “target-specific primer” refers to a primer that hybridizes selectively and predictably to a target sequence, for example a subsequence of one of the six genes disclosed herein, in a target nucleic acid sample. A target-specific primer can be selected or synthesized to be complementary to known nucleotide sequences of target nucleic acids.

The term “random primer” refers to a primer having an arbitrary sequence. The nucleotide sequence of a random primer can be known, although such sequence is considered arbitrary in that it is not specifically designed for complementarity to a nucleotide sequence of the presently disclosed subject matter. The term “random prime(encompasses selection of an arbitrary sequence having increased probability to be efficiently utilized in an amplification reaction. For example, the Random Oligonucleotide Construction Kit (ROCK) is a macro-based program that facilitates the generation and analysis of random oligonucleotide primers (Strain & Chmielewski, 2001). Representative primers include but are not limited to random hexamers and rapid amplification of polymorphic DNA (RAPD)-type primers as described by Williams et al., 1990.

A random primer can also be degenerate or partially degenerate as described by Telenius et al., 1992. Briefly, degeneracy can be introduced by selection of alternate oligonucleotide sequences that can encode a same amino acid sequence.

In some embodiments, random primers can be prepared by shearing or digesting a portion of the template nucleic acid, sample. Random primers so-constructed comprise a sample-specific set of random primers.

The term “heterologous primer” refers to a primer complementary to a sequence that has been introduced into the template nucleic acid pool. For example, a primer that is complementary to a linker or adaptor, as described below, is a heterologous primer. Representative heterologous primers can optionally include a poly(dT) primer, a poly(T) primer, or as appropriate, a poly(dA) or poly(A) primer.

The term “primer” as used herein refers to a contiguous sequence comprising in some embodiments about 6 or more nucleotides, in some embodiments about 10-20 nucleotides (e.g., 15-mer), and in some embodiments about 20-30 nucleotides (e.g., a 22-mer). Primers used to perform the methods of the presently disclosed subject matter encompass oligonucleotides of sufficient length and appropriate sequence so as to provide initiation of polymerization on a nucleic acid molecule.

U.S. Pat. No. 6,066,457 to Hampson et al. describes a method for substantially uniform amplification of a collection of single stranded nucleic acid molecules such as RNA. Briefly, the nucleic acid starting material is anchored and processed to produce a mixture of directional shorter random size DNA molecules suitable for amplification of the sample.

In accordance with the methods and systems of the presently disclosed subject matter, any PCR technique or related technique can be employed to perform the step of amplifying the nucleic acid sample. In addition, such methods can be optimized for amplification of a particular subset of nucleic acid (e.g., genomic DNA versus RNA), and representative optimization criteria and related guidance can be found in the art. See Cha & Thilly, 1993; Linz et al., 1990; Robertson & Walsh-Weller, 1998; Roux 1995; Williams 1989; and McPherson et al., 1995.

VII.C.3. Labeling of Nucleic Acid Samples

Optionally, a nucleic acid sample (e.g., a quantitatively amplified RNA sample) further comprises a detectable label. In some embodiments of the presently disclosed subject matter, the amplified nucleic acids can be labeled prior to hybridization to an array. Alternatively, randomly amplified nucleic acids are hybridized with a set of probes, without prior labeling of the amplified nucleic acids. For example, an unlabeled nucleic acid in the biological sample can be detected by hybridization to a labeled probe. In some embodiments, both the randomly amplified nucleic acids and the one or more probes include a label, wherein the proximity of the labels following hybridization enables detection. An exemplary procedure using nucleic acids labeled with chromophores and fluorophores to generate detectable photonic structures is described in U.S. Pat. No. 6,162,603 to Heller.

In accordance with the methods and systems of the presently disclosed subject matter, the amplified nucleic acids and/or probes/probe sets can be labeled using any detectable label. It will be understood to one of skill in the art that any suitable method for labeling can be used, and no particular detectable label or technique for labeling should be construed as a limitation of the disclosed methods.

Direct labeling techniques include incorporation of radioisotopic or fluorescent nucleotide analogues into nucleic acids by enzymatic synthesis in the presence of labeled nucleotides or labeled PCR primers. A radio-isotopic label can be detected using autoradiography or phosphorimaging. A fluorescent label can be detected directly using emission and absorbance spectra that are appropriate for the particular label used. Any detectable fluorescent dye can be used, including but not limited to FITC (fluorescein isothiocyanate), FLUOR X™, ALEXA FLUOR® 488, OREGON GREEN® 488, 6-JOE (6-carboxy-4′,5′-dichloro-2′,7′-dimethoxyfluorescein, succinimidyl ester), ALEXA FLUOR® 532, Cy3, ALEXA FLUOR® 546, TMR (tetramethylrhodamine), ALEXA FLUOR® 568, ROX (X-rhodamine), ALEXA FLUOR® 594, TEXAS RED®, BODIPY® 630/650, and Cy5 (available from Amersham Pharmacia Biotech of Piscataway, N.J., United States of America or from Molecular Probes Inc. of Eugene, Oreg., United States of America). Fluorescent tags also include sulfonated cyanine dyes (available from Li-Cor, Inc. of Lincoln, Nebr., United States of America) that can be detected using infrared imaging. Methods for direct labeling of a heterogeneous nucleic acid sample are known in the art and representative protocols can be found in, for example, DeRisi et al., 1996; Sapolsky & Lipshutz, 1996; Schena et al., 1995; Schena et al., 1996; Shalon et al., 1996; Shoemaker et al., 1996; and Wang et al., 1989.

In some embodiments, nucleic acid molecules isolated from different cell types (e.g., primary versus metastatic PDAC) are labeled with different detectable markers, allowing the nucleic acids to be analyzed simultaneously on an array. For example, a first RNA sample can be reverse transcribed into cDNAs labeled with cyanine 3 (a green dye fluorophore; Cy3) while a second RNA sample to which the first RNA sample is to be compared can be labeled with cyanine 5 (a red dye fluorophore; Cy5).

The quality of probe or nucleic acid sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients (Randolph & Waggoner, 1995). Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al., 2000. Thus, it will be understood to one of skill in the art that labeling methods can be optimized for performance in microarray hybridization assay, and that optimal labeling can be unique to each label type.

VII.D. Forming High-density Arrays

In some embodiments of the presently disclosed subject matter, probes or probe sets are immobilized on a solid support such that a position on the support identifies a particular probe or probe set. In the case of a probe set, constituent probes of the probe set can be combined prior to placement on the solid support or by serial placement of constituent probes at a same position on the solid support.

A microarray can be assembled using any suitable method known to one of skill in the art, and any one microarray configuration or method of construction is not considered to be a limitation of the presently disclosed subject matter. Representative microarray formats that can be used in accordance with the methods of the presently disclosed subject matter are described herein below and include, but are not limited to light-directed chemical coupling, and mechanically directed coupling (see U.S. Pat. No. 5,143,854 to Pirrung et al.; U.S. Pat. No. 5,800,992 to Fodor et al.; and U.S. Pat. NO. 5,837,832 to Chee et aL).

VII.D.1. Array Substrate and Configuration

The substrate for printing the array should be substantially rigid and amenable to DNA immobilization and detection methods (e.g., in the case of fluorescent detection, the substrate must have low background fluorescence in the region of the fluorescent dye excitation wavelengths). The substrate can be nonporous or porous as determined most suitable for a particular application. Representative substrates include but are not limited to a glass microscope slide, a glass coverslip, silicon, plastic, a polymer matrix, an agar gel, a polyacrylamide gel, and a membrane, such as a nylon, nitrocellulose or ANAPORE™ (Whatman of Maidstone, United Kingdom) membrane.

Porous substrates (membranes and polymer matrices) are preferred in that they permit immobilization of relatively large amount of probe molecules and provide a three-dimensional hydrophilic environment for biomolecular interactions to occur (Dubiley et al., 1997; Yershov et al., 1996). A BIOCHIP ARRAYER™ dispenser (Packard Instrument Company of Meriden, Conn., United States of America) can effectively dispense probes onto membranes such that the spot size is consistent among spots whether one, two, or four droplets were dispensed per spot (Englert, 2000).

A microarray substrate for use in accordance with the methods of the presently disclosed subject matter can have either a two-dimensional (planar) or a three-dimensional (non-planar) configuration. An exemplary three-dimensional microarray is the FLOW-THRU™ chip (Gene Logic, Inc. of Gaithersburg, Md., United States of America), which has implemented a gel pad to create a third dimension. Such a three-dimensional microarray can be constructed of any suitable substrate, including glass capillary, silicon, metal oxide filters, or porous polymers. See Yang et al., 1998.

Briefly, a FLOW-THRU™ chip (Gene Logic, Inc.) comprises a uniformly porous substrate having pores or microchannels connecting upper and lower faces of the chip. Probes are immobilized on the walls of the microchannels and a hybridization solution comprising sample nucleic acids can flow through the microchannels. This configuration increases the capacity for probe and target binding by providing additional surface relative to two-dimensional arrays. See U.S. Pat. No. 5,843,767 to Beattie.

VII.D.2. Surface Chemistry

The particular surface chemistry employed is inherent in the microarray substrate and substrate preparation. Probe immobilization of nucleic acids probes post-synthesis can be accomplished by various approaches, including adsorption, entrapment, and covalent attachment. Typically, the binding technique is designed to not disrupt the activity of the probe.

For substantially permanent immobilization, covalent attachment is generally performed. Since few organic functional groups react with an activated silica surface, an intermediate layer is advisable for substantially permanent probe immobilization. Functionalized organosilanes can be used as such an intermediate layer on glass and silicon substrates (Liu & Hlady, 1996; Shriver-Lake 1998). A hetero-bifunctional cross-linker requires that the probe have a different chemistry than the surface, and is preferred to avoid linking reactive groups of the same type. A representative hetero-bifunctionai cross-linker comprises gamma-maleimidobutyryloxy-succimide (GMBS) that can bind maleimide to a primary amine of a probe. Procedures for using such linkers are known to one of skill in the art and are summarized in Hermanson, 1990. A representative protocol for covalent attachment of DNA to silicon wafers is described by O'Donnell et al., 1997.

When using a glass substrate, the glass should be substantially free of debris and other deposits and have a substantially uniform coating.

Pretreatment of slides to remove organic compounds that can be deposited during their manufacture can be accomplished, for example, by washing in hot nitric acid. Cleaned slides can then be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner, 1997; Schena et al., 1995). See also Worley et al., 2000.

For attachment of probes greater than about 300 base pairs, noncovalent binding is suitable. A representative technique for noncovalent linkage involves use of sodium isothiocyanate (NaSCN) in the spotting solution. When using this method, amino-silanized slides are typically employed because this coating improves nucleic acid binding when compared to bare glass. This method works well for spotting applications that use about 100 ng/μl (Worley et al., 2000).

In the case of nitrocellulose or nylon membranes, the chemistry of nucleic acid binding chemistry to these membranes has been well characterized (Southern, 1975; Sambrook & Russell, 2001).

VII.D.3. Arraying Techniques

A microarray for the analysis of gene expression in a biological sample can be constructed using any one of several methods available in the art, including but not limited to photolithographic and microfluidic methods, further described herein below. In some embodiments, the method of construction is flexible, such that a microarray can be tailored for a particular purpose.

As is standard in the art, a technique for making a microarray should create consistent and reproducible spots. Each spot is preferably uniform, and appropriately spaced away from other spots within the configuration. A solid support for use in the presently disclosed subject matter comprises in some embodiments about 10 or more spots, in some embodiments about 100 or more spots, in some embodiments about 1,000 or more spots, and in some embodiments about 10,000 or more spots. In some embodiments, the volume deposited per spot is about 10 picoliters to about 10 nanoliters, and in some embodiments about 50 picoliters to about 500 picoliters. The diameter of a spot is in some embodiments about 50 μm to about 1000 μm, and in some embodiments about 100 μm to about 250 μm.

Light-directed synthesis. This technique was developed by Fodor et al. (Fodor et al., 1991; Fodor et al., 1993), and commercialized by Affymetrix of Santa Clara, Calif., United States of America. Briefly, the technique uses precision photolithographic masks to define the positions at which single, specific nucleotides are added to growing single-stranded nucleic acid chains. Through a stepwise series of defined nucleotide additions and light-directed chemical linking steps, high-density arrays of defined oligonucleotides are synthesized on a solid substrate. A variation of the method, called Digital Optical Chemistry, employs mirrors to direct light synthesis in place of photolithographic masks (PCT International Patent Application Publication No. WO 99/63385). This approach is generally limited to probes of about 25 nucleotides in length or less. See also Warrington et al., 2000.

Contact Printing. Several procedures and tools have been developed for printing microarrays using rigid pin tools. In surface contact printing, the pin tools are dipped into a sample solution, resulting in the transfer of a small volume of fluid onto the tip of the pins. Touching the pins or pin samples onto a microarray surface leaves a spot, the diameter of which is determined by the surface energies of the pin, fluid, and microarray surface. Typically, the transferred fluid comprises a volume in the nanoliter or picoliter range.

One common contact printing technique uses a solid pin replicator. A replicator pin is a tool for picking up a sample from one stationary location and transporting it to a defined location on a solid support. A typical configuration for a replicating head is an array of solid pins, generally in an 8×12 format, spaced at 9-mm centers that are compatible with 96- and 384-well plates. The pins are dipped into the wells, lifted, moved to a position over the microarray substrate, lowered to touch the solid support, whereby the sample is transferred. The process is repeated to complete transfer of all the samples. See Maier et al., 1994. A recent modification of solid pins involves the use of solid pin tips having concave bottoms, which print more efficiently than flat pins in some circumstances. See Rose, 2000.

Solid pins for microarray printing can be purchased, for example, from TeleChem International, Inc. of Sunnyvale, Calif. in a wide range of tip dimensions. The CHIPMAKER™ and STEALTH™ pins from TeleChem contain a stainless steel shaft with a fine point. A narrow gap is machined into the point to serve as a reservoir for sample loading and spotting. The pins have a loading volume of 0.2 μl to 0.6 μl to create spot sizes ranging from 75 μm to 360 μm in diameter.

To permit the printing of multiple arrays with a single sample loading, quill-based array tools, including printing capillaries, tweezers, and split pins have been developed. These printing tools hold larger sample volumes than solid pins and therefore allow the printing of multiple arrays following a single sample loading. Quill-based arrayers withdraw a small volume of fluid into a depositing device from a microwell plate by capillary action. See Schena et al., 1995. The diameter of the capillary typically ranges from about 10 μm to about 100 μm. A robot then moves the head with quills to the desired location for dispensing. The quill carries the sample to all spotting locations, where a fraction of the sample is deposited. The forces acting on the fluid held in the quill must be overcome for the fluid to be released. Accelerating and then decelerating by impacting the quill on a microarray substrate accomplishes fluid release. When the tip of the quill hits the solid support, the meniscus is extended beyond the tip and transferred onto the substrate. Carrying a large volume of sample fluid minimizes spotting variability between arrays. Because tapping on the surface is required for fluid transfer, a relatively rigid support, for example a glass slide, is appropriate for this method of sample delivery.

A variation of the pin printing process is the PIN-AND-RING™ technique developed by Genetic MicroSystems Inc. of Woburn, Mass., United States of America. This technique involves dipping a small ring into the sample well and removing it to capture liquid in the ring. A solid pin is then pushed through the sample in the ring, and the sample trapped on the flat end of the pin is deposited onto the surface. See Mace et al., 2000. The PIN-AND-RING™ technique is suitable for spotting onto rigid supports or soft substrates such as agar, gels, nitrocellulose, and nylon. A representative instrument that employs the PIN-AND-RING™ technique is the 417™ Arrayer available from Affymetrix of Santa Clara, Calif., United States of America.

Additional procedural considerations relevant to contact printing methods, including array layout options, print area, print head configurations, sample loading, preprinting, microarray surface properties, sample solution properties, pin velocity, pin washing, printing time, reproducibility, and printing throughput are known in the art, and are summarized by Rose, 2000.

Noncontact Ink-Jet Printing. A representative method for noncontact ink-jet printing uses a piezoelectric crystal closely apposed to the fluid reservoir. One configuration places the piezoelectric crystal in contact with a glass capillary that holds the sample fluid. The sample is drawn up into the reservoir and the crystal is biased with a voltage, which causes the crystal to deform, squeeze the capillary, and eject a small amount of fluid from the tip. Piezoelectric pumps offer the capability of controllable, fast jetting rates and consistent volume deposition. Most piezoelectric pumps are unidirectional pumps that need to be directly connected, for example by flexible capillary tubing, to a source of sample supply or wash solution. The capillary and jet orifices should be of sufficient inner diameter so that molecules are not sheared. The void volume of fluid contained in the capillary typically ranges from about 100 μl to about 500 μl and generally is not recoverable. See U.S. Pat. No. 5,965,352 to Stoughton & Friend.

Devices that provide thermal pressure, sonic pressure, or oscillatory pressure on a liquid stream or surface can also be used for ink-jet printing. See Theriault et al., 1999.

Syringe-Solenoid Printing. Syringe-solenoid technology combines a syringe pump with a microsolenoid valve to provide quantitative dispensing of nanoliter sample volumes. A high-resolution syringe pump is connected to both a high-speed microsolenoid valve and a reservoir through a switching valve. For printing microarrays, the system is filled with a system fluid, typically water, and the syringe is connected to the microsolenoid valve. Withdrawing the syringe causes the sample to move upward into the tip. The syringe then pressurizes the system such that opening the microsolenoid valve causes droplets to be ejected onto the surface. With this configuration, a minimum dispense volume is on the order of 4 nl to 8 nl. The positive displacement nature of the dispensing mechanism creates a substantially reliable system. See U.S. Pat. Nos. 5,743,960 and 5,916,524, both to Tisone.

Electronic Addressing. This method involves placing charged molecules at specific positions on a blank microarray substrate, for example a NANOCHIP™ substrate (Nanogen Inc. of San Diego, Calif., United States of America). A nucleic acid probe is introduced to the microchip, and the negatively-charged probe moves to the selected charged position, where it is concentrated and bound. Serial application of different probes can be performed to assemble an array of probes at distinct positions. See U.S. Pat. No. 6,225,059 to Ackley et al. and PCT International Patent Application Publication No. WO 01/23082.

Nanoelectrode Synthesis. An alternative array that can also be used in accordance with the methods of the presently disclosed subject matter provides ultra-small structures (nanostructures) of a single or a few atomic layers synthesized on a semiconductor surface such as silicon. The nanostructures can be designed to correspond precisely to the three-dimensional shape and electro-chemical properties of molecules, and thus can be used to recognize nucleic acids of a particular nucleotide sequence. See U.S. Pat. No. 6,123,819 to Peeters.

In brief, the light-directed combinatorial synthesis of oligonucleotide arrays on a glass surface proceeds using automated phosphoramidite chemistry and chip masking techniques. In some embodiments, a glass surface is derivatized with a silane reagent containing a functional group, e.g., a hydroxyl or amine group blocked by a photolabile protecting group. Photolysis through a photolithogaphic mask is used selectively to expose functional groups that are then ready to react with incoming 5′ photoprotected nucleoside phosphoramidites. The phosphoramidites react only with those sites that are illuminated (and thus exposed by removal of the photolabile blocking group). Thus, the phosphoramidites only add to those areas selectively exposed from the preceding step. These steps are repeated until the desired array of sequences has been synthesized on the solid surface. Combinatorial synthesis of different oligonucleotide analogues at different locations on the array is determined by the pattern of illumination during synthesis and the order of addition of coupling reagents.

In addition to the foregoing, other methods that can be used to generate an array of oligonucleotides on a single substrate are described in PCT International Patent Application Publication WO 93/09668. High-density nucleic acid arrays can also be fabricated by depositing pre-made and/or natural nucleic acids in predetermined positions. Synthesized or natural nucleic acids are deposited on specific locations of a substrate by light directed targeting and oligonucleotide directed targeting. A dispenser that moves from region to region to deposit nucleic acids in specific spots can also be employed.

VII.E. Hybridization

VII.E.1. General Considerations

The terms “specifically hybridizes” and “selectively hybridizes” each refer to binding, duplexing, or hybridizing of a molecule only to a particular nucleotide sequence under stringent conditions when that sequence is present in a complex nucleic acid mixture (e.g., total cellular DNA or RNA).

The phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen, 1993. In general, a signal to noise ratio of 2-fold (or higher) than that observed for a negative control probe in a same hybridization assay indicates detection of specific or substantial hybridization.

VII.E.2. Hybridization on a Solid Support

In some embodiments of the presently disclosed subject matter, an amplified and/or labeled nucleic acid sample is hybridized to specific probes or probe sets that are immobilized on a continuous solid support comprising a plurality of identifying positions. Representative formats of such solid supports are described herein.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5 M NaPO₄, 1 mm ethylene diamine tetraacetic acid (EDTA), 1% BSA at 50° C. followed by washing in 2×SSC, 0.1% SDS at 50° C.; in another example, a probe and target sequence hybridize in 7% SDS, 0.5 M NaPO₄, 1 mm EDTA, 1% BSA at 50° C. followed by washing in 1×SSC, 0.1% SDS at 50° C.; in another example, a probe and target sequence hybridize in 7% SDS, 0.5 M NaPO₄, 1 mm EDTA, 1% BSA at 50° C. followed by washing in 0.5×SSC, 0.1% SDS at 50° C.; in another example, a probe and target sequence hybridize in 7% SDS, 0.5 M NaPO₄, 1 mm EDTA, 1% BSA at 50° C. followed by washing in 0.1×SSC, 0.1% SDS at 50° C.; in yet another example, a probe and target sequence hybridize in 7% SDS, 0.5 M NaPO₄, 1 mm EDTA, 1% BSA at 50° C. followed by washing in 0.1×SSC, 0.1% SDS at 65° C. In some embodiments, hybridization conditions comprise hybridization in a roller tube for at least 12 hours at 42° C. in each of the above conditions, the sodium phosphate hybridization buffer can be replaced by a hybridization buffer comprising 6×SSC (or 6×SSPE), 5× Denhardt's reagent, 0.5% SDS, and 100 g/ml carrier DNA, including 0-50% formamide, with hybridization and wash temperatures chosen based upon the desired stringency. Other hybridization and wash conditions are known to those of skill in the art (see also Sambrook & Russell, 2001; Ausubel et al., 2002; and Ausubel et al., 2003; each of which is incorporated herein in its entirety). As is known in the art, the addition of formamide in the hybridization solution reduces the Tₘ by about 0.4° C. Thus, high stringency conditions include the use of any of the above solutions and 0% formamide at 65° C., or any of the above solutions plus 50% formamide at 42° C.

For some high-density glass-based microarray experiments, hybridization at 65° C. is too stringent for typical use, at least in part because the presence of fluorescent labels destabilizes the nucleic acid duplexes (Randolph & Waggoner, 1995). Alternatively, hybridization can be performed in a formamide-based hybridization buffer as described in Piétu et al., 1996.

A microarray format can be selected for use based on its suitability for electrochemical-enhanced hybridization. Provision of an electric current to the microarray, or to one or more discrete positions on the microarray facilitates localization of a target nucleic acid sample near probes immobilized on the microarray surface. Concentration of target nucleic acid near arrayed probe accelerates hybridization of a nucleic acid of the sample to a probe. Further, electronic stringency control allows the removal of unbound and nonspecifically bound DNA after hybridization. See U.S. Pat. Nos. 6,017,696 to Heller and U.S. Pat. No. 6,245,508 to Heller & Sosnowski.

II.E.3. Hybridization in Solution

In some embodiments of the presently disclosed subject matter, an amplified and/or labeled nucleic acid sample is hybridized to one or more probes in solution. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5 M NaCl at 65° C. An example of stringent wash conditions is 15 minutes in 0.2×SSC buffer at 65° C. (see Sambrook and Russell, 2001, for a description of SSC buffer). A high stringency wash can be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. Stringent conditions can also be achieved with the addition of destabilizing agents such as formamide.

For short probes (e.g., about 10 to 50 nucleotides), stringent conditions typically involve salt concentrations of less than about 1 M Na⁺ion, typically about 0.01 M to 1 M Na⁺ion concentration (or other salts) at pH 7.0-8.3, and the temperature is typically at least about 30° C.

Optionally, nucleic acid duplexes or hybrids can be captured from the solution for subsequent analysis, including detection assays. For example, in a simple assay, a single probe set is hybridized to an amplified and labeled RNA sample derived from a target nucleic acid sample. Following hybridization, an antibody that recognizes DNA:RNA hybrids is used to precipitate the hybrids for subsequent analysis. The presence of a hybrid is determined by detection of the label in the precipitate.

Alternate capture techniques can be used as will be understood to one of skill in the art, for example, purification by a metal affinity column when using probes comprising a histidine tag. As another example, the hybridized sample can be hydrolyzed by alkaline treatment wherein the double-stranded hybrids are protected while non-hybridizing single-stranded template and excess probe are hydrolyzed. The hybrids are then collected using any nucleic acid purification technique for further analysis.

To assess the expression of multiple genes and/or samples from multiple different sources simultaneously, probes or probe sets can be distinguished by differential labeling of probes or probe sets. Alternatively, probes or probe sets can be spatially separated in different hybridization vessels.

In some embodiments, a probe or probe set having a unique label is prepared for each gene or source to be detected. For example, a first probe or probe set can be labeled with a first fluorescent label, and a second probe or probe set can be labeled with a second fluorescent label. Multi-labeling experiments should consider label characteristics and detection techniques to optimize detection of each label. Representative first and second fluorescent labels are Cy3 and Cy5 (Amersham Pharmacia Biotech of Piscataway, N.J., United States of America), which can be analyzed with good contrast and minimal signal leakage.

A unique label for each probe or probe set can further comprise a labeled microsphere to which a probe or probe set is attached. A representative system is LabMAP (Luminex Corporation of Austin, Tex., United States of America). Briefly, LabMAP (Laboratory Multiple Analyte Profiling) technology involves performing molecular reactions, including hybridization reactions, on the surface of color-coded microscopic beads called microspheres. When used in accordance with the methods of the presently disclosed subject matter, an individual probe or probe set is attached to beads having a single color-code such that they can be identified throughout the assay. Successful hybridization is measured using a detectable label of the amplified nucleic acid sample, wherein the detectable label can be distinguished from each color-code used to identify individual microspheres. Following hybridization of the randomly amplified, labeled nucleic acid sample with a set of microspheres comprising probe sets, the hybridization mixture is analyzed to detect the signal of the color-code as well as the label of a sample nucleic acid bound to the microsphere. See Vignali 2000; Smith et al., 1998; and PCT International Patent Application Publication Nos. WO 01/13120; WO 01/14589; WO 99/19515; WO 99/32660; and WO 97/14028.

VII.F. Detection

Methods and systems for detecting hybridization are typically selected according to the label employed.

In the case of a radioactive label (e.g., ³²P-dNTP) detection can be accomplished by autoradiography or by using a phosphorimager as is known to one of skill in the art. In some embodiments, a detection method can be automated and is adapted for simultaneous detection of numerous samples.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al., 1996.

In some embodiments, a nucleic acid sample or probe is labeled with far infrared, near infrared, or infrared fluorescent dyes. Following hybridization, the mixture of nucleic acids and probes is scanned photoelectrically with a laser diode and a sensor, wherein the laser scans with scanning light at a wavelength within the absorbance spectrum of the fluorescent label, and light is sensed at the emission wavelength of the label. See U.S. Pat. No. 6,086,737 to Patonay et al.; U.S. Pat. No. 5,571,388 to Patonay et al.; U.S. Pat. No. 5,346,603 to Middendorf & Brumbaugh; U.S. Pat. No. 5,534,125 to Middendorf et al.; U.S. Pat. No. 5,360,523 to Middendorf et al.; U.S. Pat. No. 5,230,781 to Middendorf & Patonay; U.S. Pat. No. 5,207,880 to Middendorf & Brumbaugh; and U.S. Pat. No. 4,729,947 to Middendorf & Brumbaugh. An ODYSSEY™ infrared imaging system (Li-Cor, Inc. of Lincoln, Nebr., United States of America) can be used for data collection and analysis.

If an epitope label has been used, a protein or compound that binds the epitope can be used to detect the epitope. For example, an enzyme-linked protein can be subsequently detected by development of a colorimetric or luminescent reaction product that is measurable using a spectrophotometer or luminometer, respectively.

In some embodiments, INVADER® technology (Third Wave Technologies of Madison, Wis., United States of America) is used to detect target nucleic acid/probe complexes. Briefly, a nucleic acid cleavage site (such as that recognized by a variety of enzymes having 5′ nuclease activity) is created on a target sequence, and the target sequence is cleaved in a site-specific manner, thereby indicating the presence of specific nucleic acid sequences or specific variations thereof. See U.S. Pat. No. 5,846,717 to Brow et al.; U.S. Pat. No. 5,985,557 to Prudent et al.; U.S. Pat. No. 5,994,069 to Hall at al.; U.S. Pat. No. 6,001,567 to Brow et al.; and U.S. Pat. No. 6,090,543 to Prudent et al.

In some embodiments, target nucleic acid/probe complexes are detected using an amplifying molecule, for example a poly-dA oligonucleotide as described by Lisle et al., 2001. Briefly, a tethered probe is employed against a target nucleic acid having a complementary nucleotide sequence. A target nucleic acid having a poly-dT sequence, which can be added to any nucleic acid sequence using methods known to one of skill in the art, hybridizes with an amplifying molecule comprising a poly-dA oligonucleotide. Short oligo-dT₄₀ signaling moieties are labeled with any suitable label (e.g., fluorescent, chemiluminescent, radioisotopic labels). The short oligo-dT₄₀ signaling moieties are subsequently hybridized along the molecule, and the label is detected.

The presently disclosed subject matter also envisions use of electrochemical technology for detecting a nucleic acid hybrid according to the disclosed method. In this case, the detection method relies on the inherent properties of DNA, and thus a detectable label on the target sample or the probe/probe set is not required. In some embodiments, probe-coupled electrodes are multiplexed to simultaneously detect multiple genes using any suitable microarray or multiplexed liquid hybridization format. To enable detection, gene-specific and control probes are synthesized with substitution of the non-physiological nucleic acid base inosine for guanine, and subsequently coupled to an electrode. Following hybridization of a nucleic acid sample with probe-coupled electrodes, a soluble redox-active mediator (e.g., ruthenium 2,2′-bipyridine) is added, and a potential is applied to the sample. In the absence of guanine, each mediator is oxidized only once. However, when a guanine-containing nucleic acid is present, by virtue of hybridization of a sample nucleic acid molecule to the probe, a catalytic cycle is created that results in the oxidation of guanine and a measurable current enhancement. See U.S. Pat. No. 6,127,127 to Eckhardt et al.; U.S. Pat. No. 5,968,745 to Thorp et al.; and U.S. Pat. No. 5,871,918 to Thorp at al.

Surface plasmon resonance spectroscopy can also be used to detect hybridization. See e.g., Heaton et al., 2001; Nelson etal., 2001; and Guedon et al., 2000.

VII.G. Data Analysis

Databases and software designed for use with microarrays is discussed in U.S. Pat. No. 6,229,911 to Balaban & Aggarwal, a computer-implemented method for managing information, stored as indexed tables, collected from small or large numbers of microarrays, and U.S. Pat. No. 6,185,561 to Balaban & Khurqin, a computer-based method with data mining capability for collecting gene expression level data, adding additional attributes and reformatting the data to produce answers to various queries. U.S. Pat. No. 5,974,164 to Chee, disclose a software-based method for identifying mutations in a nucleic acid sequence based on differences in probe fluorescence intensities between wild type and mutant sequences that hybridize to reference sequences.

Analysis of microarray data can also be performed using the method disclosed in Tusher et al., 2001, which describes the Significance Analysis of Microarrays (SAM) method for determining significant differences in gene expression among two or more samples.

### VIII. Devices, Systems, and Compositions for Use in the Presently Disclosed Methods

The presently disclosed subject matter also provides devices, systems, and compositions that can be employed in the practice of the methods disclosed herein.

The methods and systems disclosed herein relate in some embodiments to generating gene expression profiles from biological samples that comprise PDAC cells obtained from a subject. The gene expression profiles are then in some embodiments compared to standards such as, but not limited to gene expression profiles of metastatic PDAC cells and/or primary (i.e., non-metastatic) PDAC cells. This comparison permits a physician to more accurately predict the degree to which a given subject is likely to benefit from particular treatment of the PDAC, which information can then assist the subject in making informed decisions as to the course of his or her treatment.

As such, the presently disclosed methods can employ various techniques to generate the gene expression profiles required for the comparisons. See e.g., PCT International Patent Application Publication Nos. WO 2004/046098; WO 2004/110244; WO 2006/089268; WO 2007/001324; WO 2007/056332; WO 2007/070252, each of which is incorporated herein by reference in its entirety.

Generally, a gene expression profile can be generated using the following basic steps:
(1) a biological sample such as, but not limited to a PDAC biopsy or resected PDAC cells are obtained; and
(2) the expression levels of one or more (e.g., two, three, four, five, or six) of the Fos B, KLF6, NFKBIZ, ATP4A, GSG1, and SIGLEC11 genes are determined.

As is known to one of ordinary skill in the art, gene expression levels can be assayed either at the level of RNA or at the level of protein. As such, in some embodiments RNA is extracted from the biological sample and analyzed by techniques that include, but are not limited to PCR analysis (in some embodiments, quantitative reverse transcription PCR) and/or array analysis. In each case, one of ordinary skill in the art would be aware of techniques that can be employed to determine the expression level of a gene product in the biological sample.

With respect to PCR analyses, the sequences of nucleic acids that correspond to exemplary Fos B, KLF6, NFKBIZ, ATP4A, GSG1, and SIGLEC11 gene products are present within the GENBANK® database (a subset of which are also provided in the Sequence Listing), and oligonucleotide primers can be designed for the purpose of determining expression levels.

Alternatively, arrays can be produced that include single-stranded nucleic acids that can hybridize to Fos B, KLF6, NFKBIZ, ATP4A, GSG1, and SIGLEC11 gene products. Exemplary, non-limiting methods that can be used to produce and screen arrays are described in Section VII hereinabove.

Therefore, in some embodiments the presently disclosed subject matter provides arrays comprising polynucleotides that are capable of hybridizing to at least five genes selected from among Fos B, KLF6, NFKBIZ, ATP4A, GSG1, and SIGLEC11 and/or comprising specific peptide or polypeptide gene products of at least five of Fos B, KLF6, NFKBIZ, ATP4A, GSG1, and SIGLEC11.

Alternatively or in addition, gene expression can be assayed by determining the levels at which polypeptides are present in PDAC tissue. This can also be done using arrays, and exemplary methods for producing peptide and/or polypeptide arrays attached to nitrocellulose-coated glass slides (Espejo etal., 2002), alkanethiol-coated gold surfaces (Houseman et al., 2002), poly-L-lysine-treated glass slides (Haab et al., 2001), aldehyde-treated glass slides (MacBeath & Schreiber, 2000; Salisbury et al., 2002), silane-modified glass slides (Fang eta!, 2002; Seong, 2002), and nickel-treated glass slides (Zhu at al., 2001), among others, have been reported.

In some embodiments, the presently disclosed subject matter provides arrays that comprise peptides or polypeptides that are correspond to gene products from one or more of Fos B, KLF6, NFKBIZ, ATP4A, GSG1, and SIGLEC11. In these embodiments, arrays are produced from proteins isolated from PDAC tissue, and these arrays are then probed with molecules that specifically bind to the various gene products of interest, if present. Exemplary molecules that specifically bind to Fos B, KLF6, NFKBIZ, ATP4A, GSG1, or SIGLEC11 gene products include antibodies (as well as fragments and derivatives thereof that include at least one Fab fragment). Antibodies to human Fos B and KLF6 are commercially available, and antibodies that specifically bind to NFKBIZ, ATP4A, GSG1, or SIGLEC11 gene products can be produced using routine techniques.

Peptide and/or polypeptide arrays can be designed quantitatively such that the amount of each individual peptide or polypeptide is reflective of the amount of that individual peptide or polypeptide in the PDAC tissue.

Further, the arrays can be designed such that specific peptide or polypeptide gene products that correspond to one or more of the Fos B, KLF6, NFKBIZ, ATP4A, GSG1, and SIGLEC11 genes can be localized (sometimes referred to as “spotted”) on the array such that the array can be interrogated with at least one antibody that specifically binds to one of the specific peptide or polypeptide gene products.

In some embodiments, gene expression at the level of protein is assayed without isolating the relevant peptides and/or polypeptides from the PDAC cells. For example, immunohistochemistry and/or immunocytochemistry can be employed, in which the expression levels of gene products that correspond to one or more of the Fos B, KLF6, NFKBIZ, ATP4A, GSG1, and SIGLEC11 genes can be determined by incubating appropriate binding molecules to PDAC cells and/or tissue. In some embodiments, the PDAC cells and/or tissue is mounted in paraffin blocks before the immunohistochemistry and/or immunocytochemistry is performed.

As would be understood by one of ordinary skill in the art upon consideration of the present disclosure, many of the manipulations disclosed herein can be automated, and it is intended that such automation is encompassed by the presently disclosed subject matter.

### EXAMPLES

The following Examples provide further illustrative embodiments. In light of the present disclosure and the general level of skill in the art, those of skill will appreciate that the following Example is intended to be exemplary only and that numerous changes, modifications, and alterations can be employed without departing from the scope of the presently disclosed subject matter.

### Materials and Methods Employed in the Examples

Patients. PDAC samples from 15 patients with resected primary PDAC from the University of North Carolina at Chapel Hill (UNC) and 15 patients with metastatic PDAC from the University of Nebraska Medical Center Rapid Autopsy Pancreatic Program (NEB) were used for the training set. For the NEB samples, human pancreatic tumors from decedents who had previously been diagnosed with PDAC were obtained from the NEB's Tissue Bank through the Rapid Autopsy Pancreatic Program in compliance with the institutional review board (IRB). To ensure minimal degradation of tissue, organs were harvested within three hours post mortem and the specimens flash frozen in liquid nitrogen or placed in formalin for immediate fixation.

The training set included 34 patients with resected PDAC from Johns Hopkins Medical Institutions (JHMI). The independent validation cohort included 78 patients from two institutions: 48 from Northwestern Memorial

Hospital (NW) and 19 from NorthShore University HealthSystem (NSU). All ples were collected between 1999 and 2007 and flash frozen in liquid nitrogen after approval by the Institutional Review Board (IRB) of each facility. The UNC IRB approved the use of all de-identified samples. All available samples were reviewed by a single pathologist in order to confirm the presence of PDAC in the samples. De-identified data including American Joint Committee on Cancer (AJCC) tumor, node and metastasis (TNM) staging, grade or differentiation, margin status, and survival were available for the majority of patients.

RNA isolation and microarray hybridization. All RNA isolation and hybridization on Agilent human whole genome 4×44K cDNA microarrays (Agilent Technologies, Inc., Santa Clara, Calif., United States of America) were performed at UNC. RNA was extracted from macrodissected snap-frozen tumor samples using Allprep Kits (Qiagen Inc., Valencia, Calif., United States of America) and quantified using NANODROP™ spectrophotometry (Thermo Fisher Scientific Inc., Wilmington, Del., United States of America). RNA quality was assessed with the use of the Bioanalyzer 2100 (Agilent Technologies). RNA was selected for hybridization using RNA integrity number and by inspection of the 18S and 28S ribosomal RNA. Similar RNA quality was selected across samples. One microgram of RNA was used as a template for cDNA preparations and hybridized to Agilent 4×44 K whole human genome arrays (Agilent Technologies). cDNA was labeled with Cy5-dUTP and a reference control (Stratagene) was labeled with Cy3-dUTP using the Agilent (Agilent Technologies) low RNA input linear amplification kit and hybridized overnight at 65° C. to Agilent 4×44 K whole human genome arrays (Agilent Technologies). Arrays were washed and scanned using an Agilent scanner (Agilent Technologies). The data are publicly available in Gene Expression Omnibus database (GEO datasets) available from the website of the national Center for Biotechnology Information (NCBI) maintained by the national Institutes of Health of the United States (Accession No. GSE21501).

Microarray and Statistical Analysis. Aii array data were normalized using LOWESS normalization. Data were excluded for genes with poor spot quality or genes that did not have mean intensity greater than 10 for one of the two channels (green and red) in at least 70% of the experiments. The log₂ ratio of the mean red intensity over mean green intensity was calculated for each gene and underwent LOWESS normalization (Yang et al., 2002). Missing data were imputed using the k-nearest neighbors imputation (KNN) with k=10 (Troyanskaya et al., 2001). A distance weighted discrimination (DWD) was used to detect the systematic biases between the different datasets and then global adjustments made to remove these biases (Benito et al., 2004). Genes that were significantly up- or down-regulated were identified using significance analysis of microarrays (SAM; Tusher et al., 2001). Two centroids were created using the mean gene expression profile of this significant gene list from the derivation set and used to develop a single sample predictor (SSP, nearest centroid algorithm; Hu et al., 2006) for an objective classifier. After DWD, the SSP was applied to a 34-patient training set where any new sample was compared to the resected centroid and assigned by the SSP distance function to the resected centroid using (1−Pearson correlation coefficient). The X-Tile software program, which assigns a two-population log-rank value to each sample and then determines the best cut-point, was used to determine the best threshold for classifying samples into high- and low-risk categories (Camp et al., 2004). X-Tile predicted that the (1−Pearson correlation coefficient) distance of 1 would be the appropriate cut-point to stratify patients into a high- and low-risk group (p=0.006). A second independent validation cohort was then used as a test set using this predetermined cut-point to evaluate outcome.

Survival analysis was performed using the statistical software programs R, the R-package “survival,” and SPSS (SPSS, Inc., a division of IBM Corp., Somers, N.Y., United States of America). Overall survival (OS) was analyzed using the Kaplan-Meier product-limit method and the significance of the related variables was measured by the log-rank test. The Fisher exact test was used to analyze associations between two variables, the Pearson Chi-square test was used to analyze association between more than two variables. Multivariable analysis and analysis of continuous and ordinal variables were performed using the Cox proportional hazards regression method.

Tissue microarrays (TMAs). TMAs were prepared from formalin-fixed paraffin embedded tissue sections using a 2 mm punch as described (Kononen et al., 1998). The arrays contained triplicate cores of matched normal and tumor tissue as well as chronic pancreatitis tissue (when available) from each patient. 5 μm sections were prepared from each TMA block. Hematoxylin and eosin (H&E) stained slides from each TMA block were reviewed by a pathologist to ensure that normal and tumor tissues were cored accurately.

Immunohistochemistry. Slides with 5 μM sections from the paraffin embedded specimens were deparaffinized and rehydrated. The slides were then subjected to alkaline heat antigen-retrieval using 1% Tris EDTA for 20 minutes in a steamer. All slides were incubated with 3% H₂O₂ for five minutes and washed with Tris-buffered saline (TBS). The slides were further treated with protein block solution (bovine serum albumin) for 20 minutes. The sections were incubated with one of the following primary antibodies for 60 minutes at room temperature: KLF6 (Catalogue No. sc-7158) 1:150 or Fos B (102) (Catalogue No. sc-48), both from Santa Cruz Biotechnology, Inc., Santa Cruz, Calif., United States of America). Following a wash with TBS, the slides were incubated with secondary labeled Polymer-HRP anti-rabbit antibody (Dako K4002; DAKO, Carpinteria, Calif., United States of America) for 30 minutes. This was followed by a five minute incubation with the substrate-chromogen 3,3′-diaminobenzidine (DAB; Catalogue No. SK-4100 from Vector Laboratories, Inc., Burlingame, Calif., United States of America). The sections were counterstained with Harris Hematoxylin. Positive staining was defined when more than 5% of cells expressed the marker and graded from 0 (no staining) to 4 (strong staining). The results of each protein marker were then expressed as intensity (I) and proportion (P) of positive epithelial cells and the score as the product of I and P (Hoos & Cordon-Cardo, 2001; Yeh et al., 2009). All stained slides were reviewed in a blinded fashion.

### Example 1

### Patient and Tumor Characteristics

In order to study the extremes of PDAC tumor biology, a diverse set of resected PDAC specimens from patients with and without metastases was collected. As the tumor microenvironment is increasingly recognized to play a critical role in tumorigenesis (Allinen et al., 2004; Mueller & Fusenig, 2004; Comoglio & Trusolino, 2005; Troester et al., 2009), tissues were macrodissected in order to preserve the normal adjacent tissue and stroma of the tumors. The characteristics of the dataset used to derive the signature (derivation set) comprised 15 primary resected PDAC tumors (UNC1) and 15 primary tumors from patients with metastatic PDAC (NEB). The training set comprised 34 patients with primary PDAC and the independent validation test set comprised 67 patients with primary PDAC (see Tables 2 and 3). There were no differences in RNA quality between the decedent and resected PDAC samples. Available treatment data of the patients in the training and test sets are also shown. One of 15 (7%) UNC1 patients received preoperative or neoadjuvant chemotherapy and 11/15 (73%) NEB patients received chemotherapy less than 6 months prior to death. No patient in the 34-patient training set received neoadjuvant chemotherapy. Only 3% ( 2/67) of patients in the test set received neoadjuvant chemotherapy and 45% ( 30/67) of patients received postoperative or adjuvant chemotherapy.

TABLE 2
Patient. Tumor, and Treatment Characteristics in the Derivation Set

| | | |
|---|---|---|
| Demographics | NEB (n = 15) | UNC1 (n = 15) |

| Median follow up (months) | N/A | 6 | (1-35) |
|---|---|---|---|
| T Stage | | | |
| 1 | N/A | 0 | |
| 2 | N/A | 2 | (13%) |
| 3 | N/A | 12 | (80%) |
| 4 | N/A | 1 | (7%) |
| N Stage | | | |
| 0 | N/A | 7 | (47%) |
| 1 | N/A | 8 | (15%) |
| M Stage | | | |
| 0 | 0 | 15 | (100%) |
| 1 | 15 | 0 | |
| Grade | | | |
| 1 | N/A | 2 | (14%) |
| 2 | N/A | 8 | (57%) |
| 3 | N/A | 4 | (29%) |
| Margin Negative | N/A | 12 | (80%) |
| Margin Positive | N/A | 3 | (20%) |
| Neoadjuvant Therapy | | | |
| No | N/A | 14 | (93%) |
| Yes | N/A | 1 | (7%) |
| Adjuvant Therapy | | | |
| No | N/A | 11 | (73%) |
| Yes | N/A | 4 | (27%) |

Chemotherapy

| | | | |
|---|---|---|---|
| No | 3 | (20%) | N/A |
| Yes | 12 | (80%) | N/A |

| | | | |
|---|---|---|---|
| Median Survival (months) | N/A | 9 | (1-35) |

N/A: not available

TABLE 3
Patient, Tumor, and Treatment Characteristics
in the Training and Testing Sets

| | JHMI | | NW/NSU | | UNC2 | |
|---|---|---|---|---|---|---|
| | (Training Set) | | (Testing Set) | | (TMA) | |
| Demographics | (n = 15) | | (n = 67) | | (n = 50) | |
| Median follow up (mo*) | 14 | (2-54) | 17 | (2-59) | 11 | (0-51) |
| T Stage | | | | | | |
| 1 | — | | 2 | (3%) | 5 | (10%) |
| 2 | 6 | (18%) | 10 | (16%) | 8 | (16%) |
| 3 | 27 | (79%) | 51 | (81%) | 32 | (66%) |
| 4 | 1 | (3%) | — | | 4 | (8%) |
| N Stage | | | | | | |
| 0 | 2 | (6%) | 25 | (38%) | 15 | (31%) |
| 1 | 32 | (94%) | 41 | (62%) | 34 | (69%) |
| M Stage | | | | | | |
| 0 | 34 | (100%) | 67 | (100%) | 47 | (96%) |
| 1 | 0 | | 0 | | 2 | (6%) |
| Grade | | | | | | |
| 1 | 1 | (3%) | 2 | (3%) | 2 | (4%) |
| 2 | 13 | (38%) | 34 | (54%) | 26 | (54%) |
| 3 | 20 | (59%) | 27 | (43%) | 20 | (42%) |
| Margin Negative | N/A | | 51 | (80%) | 7 | (78%) |
| Margin Positive | N/A | | 13 | (20%) | 2 | (12%) |
| Neoadjuvant Therapy | | | | | | |
| No | 34 | (100%) | 65 | (97%) | 7 | (88%) |
| Yes | 0 | | 2 | (3%) | 1 | (12%) |
| Adjuvant Therapy | | | | | | |
| No | N/A | | 30 | (45%) | N/A | |
| Yes | N/A | | 37 | (55%) | N/A | |
| Median Survival (mo) | 13 | (2-54) | 21 | (3-59) | 12 | (0-51) |

N/A: not available;
*months

### Example 2

### Gene Expression Differences in Non-metastatic and Metastatic Primary Tumors

It was hypothesized that it would be possible to enrich for molecular differences in primary PDAC, which might be clinically and biologically relevant, through examining primary tumors representing opposite spectrums of PDAC: early (localized) and late (metastatic) stage. To accomplish this, non-metastatic (UNC1) and metastatic (NEB) primary PDAC tumors were compared. As the methods of procurement for these tumors differed, DWD was used to identify systematic biases between the two datasets (Benito et al., 2004). This method has been used previously to successfully combine three breast cancer datasets across three microarray platforms (Hu et al., 2006), across species (Herschkowitz et al., 2007), and across multiple datasets (Lu et al., 2006; Oh et aL, 2006). DWD was thus used to adjust for the systematic biases between the UNC1 and NEB datasets by taking advantage of the fact that each dataset also had 15 normal pancreas samples assayed. In short, DWD was used to adjust these 15 tumor-normal pairs from both datasets to have similar distributions in principal component (PC) 1×PC 2 space.

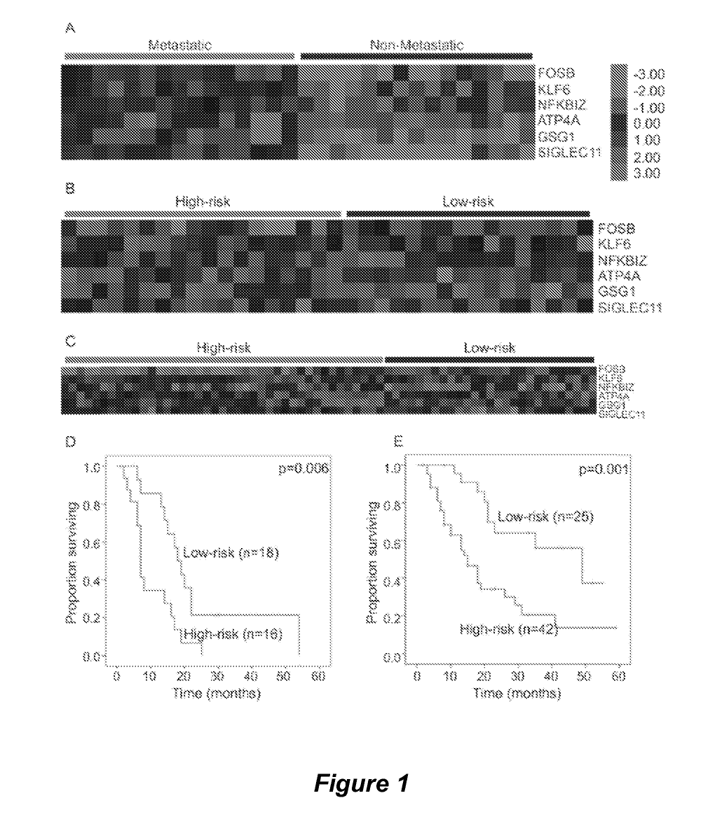

After the DWD adjustment, SAM was used to identify differentially expressed genes (Tusher et al., 2001; Yang et al., 2002). Using a false discovery rate of 5%, six genes were identified that were differentially overexpressed between non-metastatic and metastatic primary tumors: FBJ murine osteosarcoma viral oncogene homolog B (Fos B), Kruppel-like factor 6 (KLF6), nuclear factor of kappa light polypeptide gene enhancer in B-cells inhibitor, zeta (NFKBIZ, IKBZ, MAIL), ATPase H+/K+ exchanging, alpha polypeptide (ATP4A), germ cell associated 1 (GSG1), and sialic acid binding Ig-like lectin 11 (SIGLEC11; see FIG. 1A and Table 4).

TABLE 4
SAM of Metastatic Compared to Localized Primary Tumors

| Gene Name | | | | | |
|---|---|---|---|---|---|
| (GENBANK ® | Score | Numerator | Denominator | Fold | |
| Accession No.) | (d) | (r) | (s + s0) | Change | Q-value |
| Fos B | 4.34 | 1.82 | 0.42 | 2.81 | 0 |
| (NM_006732)□ | | | | | |
| GSG1 | 3.76 | 2.06 | 0.55 | 3.77 | 0 |
| (NM_031289)□ | | | | | |
| KLF6 | 3.73 | 0.99 | 0.26 | 2.10 | 0 |
| (NM_001008490) | | | | | |
| NFKBIZ | 3.38 | 0.80 | 0.24 | 1.75 | 4.74 |
| (NM_031419)□ | | | | | |
| ATP4A | 3.37 | 1.62 | 0.48 | 4.72 | 4.74 |
| (NM_000704) | | | | | |
| SIGLEC11 | 3.36 | 1.37 | 0.41 | 1.65 | 4.74 |
| (NM_052884) | | | | | |

### Example 3

### Development of a Classifier Using the Six-Gene Signature

The relationship of the presently disclosed six-gene signature to outcome was examined using a training set of 34 patients with localized and resected PDAC. After identifying and adjusting for systematic bias using DWD, a resected centroid-based predictor (Hu et al., 2006) was created using the 30 samples in the derivation dataset. The centroid was then applied to the DWD-adjusted training set of primary PDAC patients to determine the performance of the six-gene signature. X-tile (Camp et al., 2004) was used to determine the optimal distance function to the centroid cut-point for classifying this training set of patients into high-risk and low-risk groups on the basis of survival (see FIGS. 1B and 1 D). The optimal cut-point occurred at a Pearson correlation coefficient of zero (p=0.006) with patients with Pearson correlation coefficients greater than zero in the low-risk and less than zero in the high-risk groups.

### Example 4

### Application of the Six-gene Signature to an Independent Validation Cohort of 67 Patients

In order to evaluate the performance of the cut-point determined by X-tile (Camp et al., 2004), the cut-point was applied to an independent validation test set of 67 patients with primary PDAC. The predetermined Pearson correlation coefficient cut-point of zero distance to the centroid successfully stratified patients into high (n=42) and low risk groups (n=25) with a median overall survival (OS) of 15 versus 49 months (p=0.001; see FIGS. 1C and 1E). Patients in the high-risk group had 1-, 2-, and 3-year estimated survival rates of 55%, 34%, and 21%, compared to 91%, 64%, and 56% in the low-risk group.

Previous studies in PDAC have found that nodal status is the most predictive of outcome for patients with localized PDAC (Sohn et al., 2000). The prognostic signature disclosed herein was compared to current clinical prognostic benchmarks. It was determined that tumors that were node positive (p=0.091) and grade 2 or 3 trended towards a shorter survival (p=0.080). Neither T stage (p=0.977) nor margin status (p=0.223) were prognostic in this cohort. Treatment with adjuvant chemotherapy (p=0.699) or with neoadjuvant chemotherapy (p=0.409) was also not prognostic, although only two patients received neoadjuvant chemotherapy. No gene expression changes between the tumors of the two patients who received neoadjuvant chemotherapy and the tumors of patients who received no treatment prior to surgery were found.

A desirable feature of any prognostic signature is that it should be independent or additive to currently used clinicopathologic prognostic criteria. The prognostic importance of the molecular signature disclosed herein was thus compared with respect to grade (p=0.417), nodal status (p=0.381), T stage (p=0.675), and margin status (p=0.295). The six-gene signature disclosed herein was the only independent predictor of survival in the 57 patients with complete data, with a hazard ratio of 4.1 (95% confidence interval 1.7-10.0; see Table 5).

TABLE 5
Cox Proportional Hazards Regression
Analysis of the Six-Gene Signature

| Variable | Hazard Ratio | CI | p-Value |
|---|---|---|---|
| Six-gene Signature | 4.1 | 1.7-10.0 | 0.002 |
| T stage | — | — | 0.675 |
| N stage | — | — | 0.381 |
| Grade | — | — | 0.417 |
| Margin status | — | — | 0.295 |

Whether the presently disclosed six-gene signature was confounded by available clinicopathological variables was also investigated. It was determined that no association between the molecular signature and tumor size, grade, margin status, nodal status, and/or neoadjuvant or adjuvant chemotherapy was present in the independent test set (see Table 6).

TABLE 6
Relationship Between the Six-Gene Signature
And Clinicopathological Variables

| | Six-Gene Signature | | | | | |
|---|---|---|---|---|---|---|
| Variable | High Risk | | Low Risk | | p-Value | |
| T Stage | | | | | | |
| 1 | 1 | (50%) | 1 | (50%) | 0.886 | |
| 2 | 6 | (60%) | 4 | (40%) | — | |
| 3 | 33 | (65%) | 18 | (35%) | — | |
| N Stage | | | | | | |
| 0 | 13 | (52%) | 12 | (48%) | 0.203 | |
| 1 | 28 | (68%) | 13 | (32%) | — | |
| Grade | | | | | | |
| 1 | 1 | (50%) | 1 | (50%) | 0.788 | |
| 2 | 22 | (65%) | 12 | (35%) | — | |
| 3 | 19 | (70%) | 8 | (30%) | — | |
| Margin Negative | 31 | (59%) | 22 | (41%) | 0.344 | |
| Margin Positive | 9 | (75%) | 3 | (25%) | — | |
| Neoadjuvant Therapy | | | | | | |
| No | 42 | (65%) | 23 | (35%) | 0.136 | |
| Yes | 0 | | 2 | (100%) | — | |
| Adjuvant Therapy | | | | | | |
| No | 24 | (65%) | 13 | (35%) | 0.801 | |
| Yes | 18 | (60%) | 12 | (40%) | — | |
| Median Survival | 13 | (2-54) | 21 | (3-59) | 12 | (0-51) |
| (months) | | | | | | |

### Example 5

### KLF6 Expression in Primary PDAC

In order to further validate the six-gene signature, immunohistochemical analyses for KLF6 was performed, which showed a wide range of expression values between non-metastatic versus metastatic samples (see FIG. 1A). To evaluate KLF6 protein expression, another independent dataset of 50 patients represented on a TMA with matched normal, chronic pancreatitis, and PDAC was obtained (UNC2; see Table 3).

A relative scale was developed to quantify expression levels, which ranged from 0 (undetectable) to 3 (high level expression). Each sample was given a score (or a series of scores) that was calculated as the product of the intensity value attributed to a sample x the proportion of cells in the sample that were positive. For instance, a score of 1.5 would be given to a sample that included 50% positive cells with an intensity level of 3. The maximum overall score was thus 3 (100% of cells with an intensity level of 3) and the minimum score was 0.

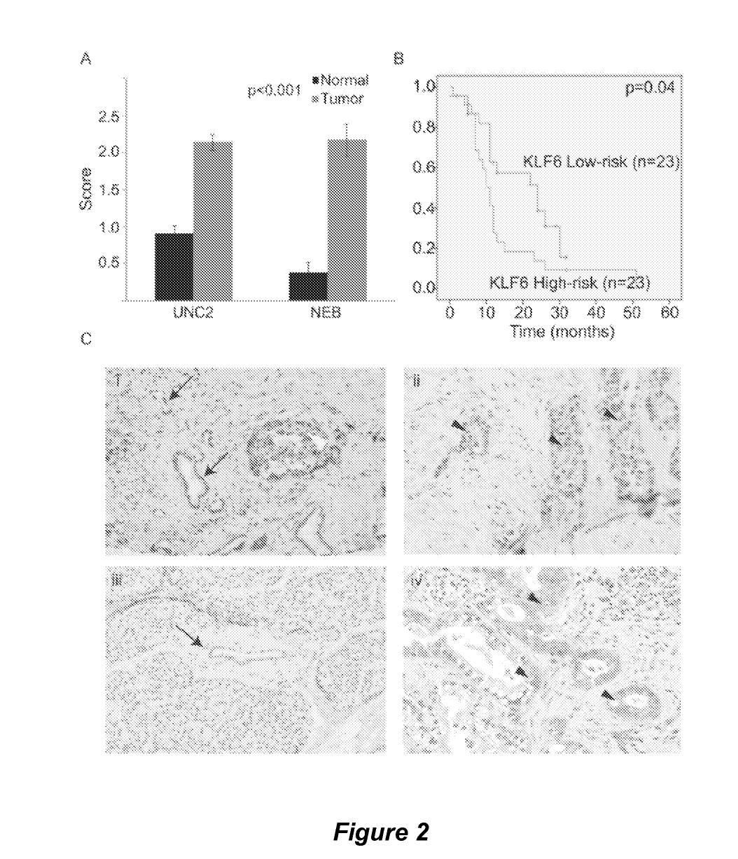

Using a median score of 1.5 as an arbitrary cutoff between “high” (i.e., a score of greater than 1.5) and “low” (i.e., a score of less than 1.5), it was determined that KLF6 expression was much higher in tumors compared to normal pancreas (p <0.001; see FIGS. 2A and 2C). KLF6 expression was strong in normal islet cells (FIG. 2C(i); white arrowhead).

Second, it was determined that KLF6 expression with a score greater than 1.5 (high) was associated with a shorter median survival of 11 months compared to 24 months for patients with KLF6 expression scores less than 1.5 (low) (p=0.04; see FIG. 2B).

Discussion of the Examples

Disclosed herein are experiments in which non-metastatic and metastatic primary PDAC tumors were profiled and compared, and an exemplary six-gene signature was identified. Although this signature was not derived on the basis of outcomes, it was demonstrated that it was prognostic in a true test set of resectable PDAC patients. The six-gene signature disclosed herein was independently predictive of survival, stratifying patients with median survival of 15 compared to 49 months, which outperforms current pathological staging criteria and indicating that the disclosed signature is likely to be a powerful prognostic tool for patients with localized PDAC.

PDAC continues to be a devastating disease with few long-term survivors. Surgery remains the standard therapy for patients diagnosed with resectable PDAC (Yeo et al., 1997). Yet, with a median survival only of less than 2 years after surgery, the attendant postoperative mortality rate of 2%-6% (Eppsteiner et al., 2009; Yermilov et al., 2009), and postoperative complication and hospital readmission rates of 59% (DeOliveira etal., 2006), the decision for surgery should be made cautiously.

Therefore, improved patient selection for therapy is needed. For the majority of patients who cannot undergo surgery, gemcitabine chemotherapy remains the best option. However, only 5%-10% of patients respond to the treatment (Abou-Alfa et al., 2006; Van Cutsem et al., 2009). Given the current therapeutic limitations, additional prognostic tools are needed to help a patient decide whether to have surgery and/or neoadjuvant chemotherapy, or when to consider participation in a clinical trial.

Disclosed herein is the discovery that a surprisingly small number of genes with differential expression between early compared to late stage primary PDAC (see Table 7) can be employed to predict treatment course and outcomes. This finding suggested that primary PDAC might be largely homogenous from a global gene expression standpoint. Nonetheless, the differences identified herein appeared to be clinically and therefore biologically important. The findings of molecular differences in resected primary PDAC tumors disclosed herein suggested that there were subtle biological variations in these tumors that influenced outcome. This presently disclosed subject matter is believed to represent the first analysis of molecular differences between non-metastatic versus metastatic primary tumors that can be employed to identify and validate prognostic signatures for PDAC.

TABLE 7
Comparison of Individual Genes in High and Low Risk Groups

| Gene | Group 1 (High Risk) | | Group 2 (Low Risk) | | |
|---|---|---|---|---|---|
| Name | Average | Stdev | Average | Stdev | p-Value* |

Johns Hopkins Medical Institutions

| | | | | | |
|---|---|---|---|---|---|
| SIGLEC11 | 0.0604 | 0.8489 | −0.3163 | 0.7000 | 0.2098 |
| KLF6 | 0.3502 | 0.8063 | 0.1471 | 0.6985 | 0.2571 |
| Fos B | −0.5505 | 0.9013 | 0.4487 | 0.9527 | 0.0033 |
| ATP4A | −0.1818 | 0.9529 | −0.1984 | 0.8164 | 0.9360 |
| NFKBIZ | 0.5668 | 0.7767 | −0.1826 | 0.7753 | 0.0010 |
| GSG1 | −0.2451 | 0.8350 | 0.1019 | 1.3196 | 0.1912 |

Northwestern Memorial Hospital/
NorthShore University Health System

| | | | | | |
|---|---|---|---|---|---|
| SIGLEC11 | 0.1145 | 0.5742 | −0.2116 | 0.7660 | 0.0492 |
| KLF6 | 0.5304 | 0.6399 | −0.2474 | 0.5880 | 0.0000 |
| Fos B | −0.9593 | 0.8241 | 0.9310 | 0.7750 | 0.0000 |
| ATP4A | 0.0885 | 0.7960 | 0.1234 | 0.8217 | 0.8619 |
| NFKBIZ | 0.1985 | 0.9682 | −0.7227 | 0.8165 | 0.0001 |
| GSG1 | 0.0270 | 0.9123 | 0.1259 | 0.8499 | 0.6548 |

*2 Sided T-Test Type 2

Of the six genes identified herein, most have not been reported to have a clear role in carcinogenesis. Three of the six genes demonstrated significantly higher expression in the poor prognostic groups (SIGLEC11, KLF6, NFKBIZ; see Table 7). ATP4A, GSG1, and SIGLEC11 do not appear to have been studied in cancer. SIGLEC11 is presently thought to be expressed by tissue macrophages and also the brain microglia (Angata et al., 2002). Interestingly, a missense mutation of SIGLEC11 (S465A) was identified in the mutation discovery screen of the recent genome-wide sequencing of PDAC (Jones et al., 2008). NFKBIZ, also called IkappaB zeta, binds to the p50 subunit of nuclear factor (NF)-kappaB and plays a role in interleukin-6 (IL-6) induction, and might be induced by IL-1 receptor and Toll-like receptors (Angata et al., 2002). Given the prevalence of chronic pancreatitis and high degree of stromal fibrosis, it is possible that NFKBIZ plays a role in PDAC and inflammation.

KLF6 is a transcription factor and its full length transcript is thought to be a tumor suppressor gene involved in prostate, lung, and ovarian carcinogenesis (DiFeo et al., 2009). However, a splice variant, KLF6-SV1, has been shown to have oncogenic properties. The oligonucleotide probes used in the Agilent whole human genome array employed herein and the antibody against KLF6 did not differentiate between the full-length and splice variant. It was found that KLF6 protein expression was higher in tumors than normal pancreas. In addition, it was determined that higher KLF6 expression was associated with poorer survival.

Only one patient in the UNCI cohort was treated with neoadjuvant chemotherapy compared to 80% of NEB patients who were treated with palliative chemotherapy. Although there is a possibility that the six-gene signature disclosed herein might be reflective of gemcitabine treatment or perhaps resistance, as NEB patients died of metastatic disease despite gemcitabine treatment, the successful application of the presently disclosed six-gene signature on an independent test set of patients where only 3% of patients with localized PDAC were treated with neoadjuvant therapy suggested that it is a rigorous predictor of prognosis in previously untreated patients. No association between the presently disclosed six-gene signature and whether a patient received adjuvant chemotherapy was identified. In addition, chemotherapy treatment in this cohort, either pre- or postoperative, did not demonstrate a survival advantage.

The exemplary six-gene signature disclosed herein was also applied to an independent dataset of 67 patients, which validated its prognostic value. In addition, the protein expression of KLF6 was validated in a 50-patient TMA. Although not nearly as powerful a predictor of prognosis as the presently disclosed six-gene signature, it was found that KLF6 expression was prognostic in the 50-patient TMA disclosed herein.

Studies of patients with resectable PDAC have demonstrated median survivals of up to 22 months, equivalent to the median survival of patients in the presently disclosed training and testing cohorts. The finding that the presently disclosed six-gene signature was able to stratify patients, with startling differences in survival, suggested that it can be used to select patients for particular therapies. For example, for patients who are at high operative risk, knowledge of a median survival of 49 compared to 15 months can be helpful in the operative decision-making process. Similarly, patients who have a poor prognosis based on the six-gene signature can be considered for neoadjuvant therapy. Currently, a minority of centers use neoadjuvant therapy as a standard of care, with most instead reserving this treatment for patients with locally advanced unresectable or borderline resectable tumors.

Therefore, the current decision-making process is based on anatomical considerations. The prognostic signature disclosed herein can refine this paradigm such that neoadjuvant therapy is offered to patients on the basis of biological considerations, regardless of resectability, and could allow for the further study and maximization of the benefits of neoadjuvant treatment. In addition, as new therapies are developed, the prognostic signature disclosed herein can help to determine whether patients might require more or less aggressive treatment.

### REFERENCES

The references listed below as well as all references cited in the specification including, but not limited to patents, patent application publications, journal articles, and database entries (e.g., GENBANK® database entries including all annotations and references cited therein) are incorporated herein by reference to the extent that they supplement, explain, provide a background for, or teach methodology, techniques, and/or compositions employed herein.
Abou-Alfa et al. (2006) Randomized phase III study of exatecan and gemcitabine compared with gemcitabine alone in untreated advanced pancreatic cancer. J Clin Oncol 24:4441-4447.
Ahmad et al. (2001) Long term survival after pancreatic resection for pancreatic adenocarcinoma. Am J Gastroenterol 96:2609-2615.
Albert et al. (1992) J Virol 66:5627-5630.
Alexay et al. (1996) The International Society of Optical Engineering 2705/63.
Allinen et al. (2004) Molecular characterization of the tumor microenvironment in breast cancer. Cancer Cell 6:17-32.
Angata et al. (2002) Cloning and characterization of human SIGLEC11. A recently evolved signaling that can interact with SHP-1 and SHP-2 and is expressed by tissue macrophages, including brain microglia. J Biol Chem 277:24466-24474.
Ausubel et al. (2002) Short Protocols in Molecular Biology, Fifth ed. Wiley, New York, N.Y., United States of America.
Ausubel et al. (2003) Current Protocols in Molecular Biology, John Wylie & Sons, Inc., New York, N.Y., United States of America.
Bej et al. (1991) Polymerase chain reaction-gene probe detection of microorganisms by using filter-concentrated samples. Appl Environ Microbiol 57:3529-3534.
Benito et al. (2004) Adjustment of systematic microarray data biases. Bioinformatics 20:105-114.
Boom et al. (1990) Rapid and simple method for purification of nucleic acids. J Clin Microbiol 28:495-503.
Boyle & Levin (2008) World Cancer Report 2008. Lyon, International Agency for Research on Cancer.
Buffone et al. (1991) Improved amplification of cytomegalovirus DNA from urine after purification of DNA with glass beads. Clin Chem 37:1945-1949.
Busch et al. (1992) Impact of specimen handling and storage on detection of hepatitis C virus RNA. Transfusion 32:420-425.
Camp et al. (2004) X-tile: a new bio-informatics tool for biomarker assessment and outcome-based cut-point optimization. Clin Cancer Res 10:7252-7259.
Cha & Thilly (1993) Specificity, efficiency, and fidelity of PCR. PCR Methods Appl 3:S18-S29.
Cousins et al. (1992) Use of polymerase chain reaction for rapid diagnosis of tuberculosis. J Clin Microbiol 30:255-258.
Cleary et al. (2004) Prognostic factors in resected pancreatic adenocarcinoma: analysis of actual 5-year survivors. J Am Coll Surg 198:722-731.
Comoglio & Trusolino (2005) Cancer: the matrix is' now in control. Nat Med 11:1156-1159.
Conlon et al. (1996) Long-term survival after curative resection for pancreatic ductal adenocarcinoma. Clinicopathologic analysis of 5-year survivors. Ann Surg 223:273-279.
DeOliveira etal. (2006) Assessment of complications after pancreatic surgery: a novel grading system applied to 633 patients undergoing pancreaticoduodenectomy. Ann Surg 244:931-937; discussion 937-939.
DeRisi et al. (1996) Use of a cDNA microarray to analyse gene expression patterns in human cancer. Nat Genet 14:457-460.
DiFeo et al. (2009) The role of KLF6 and its splice variants in cancer therapy. Drug Resist Updates 12:1-7.
Dubiley et al. (1997) Fractionation, phosphorylation and ligation on oligonucleotide microchips to enhance sequencing by hybridization. Nucl Acids Res 25:2259-2265.
Eisenberg & Levanon (2003) Human housekeeping genes are compact. Trends Genet 19:362-365.
Englert (2000) in Schena, ed., Microarray Biochip Technology, pp. 231-246, Eaton Publishing, Natick, Mass., United States of America.
Eppsteiner et al. (2009) Surgeon volume impacts hospital mortality for pancreatic resection. Ann Surg 249:635-640.
Espejo et al. (2002) A protein-domain microarray identifies novel protein-protein interactions. Biochem J 367:697-702.
Fang et al. (2002) G-protein-coupled receptor microarrays. Chembiochem 3:987-991.
Ferrone et al. (2008) Pancreatic adenocarcinoma: the actual 5-year survivors. J Gastrointest Surg 12:701-706.
Fodor et al. (1991) Light-directed, spatially addressable parallel chemical synthesis. Science 251:767-773.
Fodor et al. (1993) Multiplexed biochemical assays with biological chips. Nature 364:555-556.
GENBANK® Accession Nos. NM_{—}000704; NM_{—}001005474; NM_{—}001080554; NM_{—}001080555; NM_{—}001114171; NM_{—}001135163; NM_{—}001160124; NM_{—}001160125; NM_{—}001300; NM_{—}006732; NM_{—}031289; NM_{—}031419; NM_{—}052884; NM_{—}153823; NP_{—}000695; NP_{—}001005474; NP_{—}001074023; NP_{—}001074024; NP_{—}001107643; NP_{—}001128635; NP_{—}001153596; NP_{—}001153597.1; NP_{—}001291; NP_{—}006723; NP_{—}112579; NP_{—}113607; NP_{—}443116; NP_{—}722545.
Goggins (2007) Identifying molecular markers for the early detection of pancreatic neoplasia. Semin Oncol 34:303-310.
Grote & Logsdon (2007) Progress on molecular markers of pancreatic cancer. Curr Opin Gastroenterol 23:508-514.
Grutzmann et al. (2004) Microarray-based gene expression profiling in pancreatic ductal carcinoma: status quo and perspectives. Int J Colorectal Dis 19:401-413.
Grutzmann et al. (2005) Meta-analysis of microarray data on pancreatic cancer defines a set of commonly dysregulated genes. Oncogene 24:5079-5088.
Guedon et al. (2000) Characterization and optimization of a real-time, parallel, label-free, polypyrrole-based DNA sensor by surface plasmon resonance imaging. Anal Chem 72(24):6003-6009.
Haab et al. (2001) Protein microarrays for highly parallel detection and quantitation of specific proteins and antibodies in complex solutions. Genome Biol 2:RESEARCH0004.
Hamel et al. (1995) Rapid detection of bovine viral diarrhea virus by using RNA extracted directly from assorted specimens and a one-tube reverse transcription PCR assay. J Clin Microbiol 33:287-291.
Han et al. (2006) Analysis of long-term survivors after surgical resection for pancreatic cancer. Pancreas 32:271-275.
Hartel et al. (2008) Increased alternative splicing of the KLF6 tumour suppressor gene correlates with prognosis and tumour grade in patients with pancreatic cancer. Eur J Cancer 44:1895-1903.
Heaton et al. (2001) Electrostatic surface plasmon resonance: direct electric field-induced hybridization and denaturation in monolayer nucleic acid films and label-free discrimination of base mismatches. Proc Natl Acad Sci USA 98(7):3701-3704.
Hermanson (1990) Bioconjugate Techniques, Academic Press, San Diego, Calif., United States of America.
Herrewegh et al. (1995) Detection of feline coronavirus RNA in feces, tissues, and body fluids of naturally infected cats by reverse transcriptase PCR. J Clin Microbiol 33:684-689.
Herschkowitz et al. (2007) Identification of conserved gene expression features between murine mammary carcinoma models and human breast tumors. Genome Biol 8:R76.
Hoos & Cordon-Cardo (2001) Tissue microarray profiling of cancer specimens and cell lines: opportunities and limitations. Lab Invest 81:1331-1338.
Houseman et al. (2002) Peptide chips for the quantitative evaluation of protein kinase activity. Nat Biotechnol 20:270-274.
Hu et (2006) The molecular portraits of breast tumors are conserved across microarray platforms. BMC Genomics 7:96.
Izraeli et al. (1991) Detection of gene expression by PCR amplification of RNA derived from frozen heparinized whole blood. Nucl Acids Res 19:6051.
Jones at al. (2008) Core signaling pathways in human pancreatic cancers revealed by global genomic analyses. Science 321: 1801-1806.
Kohsaka & Carson (1994) Solid-Phase polymerase chain reaction. J Clin Lab Anal 8:452-455.
Kolbert at al. (2008) Microarray technologies for gene transcript analysis in pancreatic cancer. Technol Cancer Res Treat 7:55-59.
Kononen et al. (1998) Tissue microarrays for high-throughput molecular profiling of tumor specimens. Nat Med 4:844-847.
Lanciotti at al. (1992) Rapid detection and typing of dengue viruses from clinical samples by using reverse transcriptase-polymerase chain reaction. J Clin Microbiol 30:545-551.
Linz et al. (1990) Systematic studies on parameters influencing the performance of the polymerase chain reaction. J Clin Chem Clin Biochem 28:5-13.
Lisle et al. (2001) Novel signal amplification technology with applications in DNA and protein detection systems. Bio Techniques 30:1268-1272.
Liu & Hlady (1996) Chemical pattern on silica surface prepared by UV irradiation of 3-mercaptopropyltriethoxy silane layer: surface characterization and fibrinogen adsorption, Colloids Surfaces B Biointerfaces 8:25-37.
Lockhart et al. (1996) Expression monitoring by hybridization to high-density oligonucleotide arrays. Nat Biotechnol 14:1675-1680.
Lu at al. (2006) A gene expression signature predicts survival of patients with stage I non-small cell lung cancer. PLoS Med 3:e467.
MacBeath & Schreiber (2000) Printing proteins as microarrays for high-throughput function determination. Science 289:1760-1763.
Mace at al. (2000) in Schena, ed., Microarray Biochip Technology, pp. 39-64, Eaton Publishing, Natick, Mass., United States of America.
Maier at al. (1994) Application of robotic technology to automated sequence fingerprint analysis by oligonucleotide hybridisation. J Biotechnol 35:191-203.
McCaustland et al. (1991) Application of two RNA extraction methods prior to amplification of hepatitis E virus nucleic acid by the polymerase chain reaction. J Virol Methods 35:331-342.
McGall et al. (1996) Light-directed synthesis of high-density oligonucleotide arrays using semiconductor photoresists. Proc Nat Acad Sci USA 93:13555-13460.
McPherson et al. (1995) PCR 2: A Practical Approach, IRL Press, New York, N.Y., United States of America.
Milde-Langosch et al. (2003) FosB is highly expressed in normal mammary epithelia, but down-regulated in poorly differentiated breast carcinomas. Breast Cancer Res Treat 77:265-275.
Millar et al. (1995) Solid-phase hybridization capture of low-abundance target DNA sequences: application to the polymerase chain reaction detection of Mycobacterium paratuberculosis and Mycobacterium avium subsp. silvaticum. Anal Biochem 226:325-330.
Mueller & Fusenig (2004) Friends or foes—bipolar effects of the tumour stroma in cancer. Nat Rev Cancer 4:839-849.
Natarajan et al. (1994) An internally controlled virion PCR for the measurement of HIV-1 RNA in plasma. PCR Methods Appl 3:346-350.
Nelson et al. (2001) Surface plasmon resonance imaging measurements of DNA and RNA hybridization adsorption onto DNA microarrays. Anal Chem 73(1):1-7.
Neuhaus et al. (2008) CONKO-001: Final results of the randomized, prospective, multicenter phase III trial of adjuvant chemotherapy with gemcitabine versus observation in patients with resected pancreatic cancer (PC). Journal of Clinical Oncology, 2008 ASCO Annual Meeting Proceedings 26: LBA4504.
O'Donnell et al. (1997) High-density, covalent attachment of DNA to silicon wafers for analysis by MALDI-TOF mass spectrometry. Anal Chem 69:2438-2443.
Oh et a,. (2006) Estrogen-regulated genes predict survival in hormone receptor-positive breast cancers. J Clin Oncol 24:1656-1664.
Ohtsuka et al. (1985) An alternative approach to deoxyoligonucleotides as hybridization probes by insertion of deoxyinosine at ambiguous codon positions. J Biol Chem 260:2605-2608.
Paladichuk (1999) Isolating RNA: Pure and Simple. The Scientist 13(16):20-23.
Parkin et al. (2005) Global cancer statistics, 2002. CA Cancer J Clin 55:74-108.
PCT International Patent Application Publication Nos. WO 1993/09668; WO 1995/11755; WO 1997/14028; WO 1999/19515; WO 1999/32660; WO 1999/63385; WO 2001/13120; WO 2001/14589; WO 2001/23082; WO 2004/046098; WO 2004/110244; WO 2006/089268; WO 2007/001324; WO 2007/056332; WO 2007/070252.
Piétu et al. (1996) Novel gene transcripts preferentially expressed in human muscles revealed by quantitative hybridization of a high density cDNA array. Genome Res 6:492-503.
Randolph & Waggoner (1995) Stability, specificity and fluorescence brightness of multiply-labeled DNA probes. Nucl Acids Res 25:2923-2929.
Ratner & Castner (1997) in Vickerman, ed., Surface Analysis: The Principal Techniques, John Wiley & Sons, New York, United States of America.
Robertson & Walsh-Weller (1998) An introduction to PCR primer design and optimization of amplification reactions. Methods Mol Biol 98:121-154.
Rose (2000) in Schena, ed., Microarray Biochip Technology, pp. 19-38, Eaton Publishing, Natick, Mass., United States of America.
Roux (1995) Optimization and troubleshooting in PCR. PCR Methods Appl 4:S185-S194.
Rupp et al. (1988) Purification and analysis of RNA from paraffin-embedded tissues. Bio Techniques 6:56-60.
Salisbury et al. (2002) Peptide microarrays for the determination of protease substrate specificity. J Am Chem Soc 124:14868-14870.
Sambrook & Russell (2001) Molecular Cloning: A Laboratory Manual, Cold Spring Harbor Press, Cold Spring Harbor, N.Y.
Sapolsky & Lipshutz (1996) Mapping genomic library clones using oligonucleotide arrays. Genomics 33:445-456.
Schena et al. (1995) Quantitative monitoring of gene expression patterns with a complementary DNA microarray. Science 270:467-470.
Schena et al. (1996) Parallel human genome analysis: microarray-based expression monitoring of 1000 genes. Proc Natl Acad Sci USA 93:10614-10619.
Schnelldorfer et al. (2008) Long-term survival after pancreatoduodenectomy for pancreatic adenocarcinoma: is cure possible? Ann Surg 247:456-462.
Seong (2002) Microimmunoassay using a protein chip: optimizing conditions for protein immobilization. Clin Diagn Lab Immunol 9:927-930.
Shalon et al. (1996) A DNA microarray system for analyzing complex DNA samples using two-color fluorescent probe hybridization. Genome Res 6:639-645.
Shoemaker et al. (1996) Quantitative phenotypic analysis of yeast deletion mutants using a highly parallel molecular bar-coding strategy. Nat Genet 14:450-456.
Shriver-Lake (1998) in Cass & Ligler, eds., Immobilized Biomolecules in Analysis, pp. 1-14, Oxford Press, Oxford, United Kingdom.
Silhavy et al. (1984) Experiments with Gene Fusions, Cold Spring Harbor Laboratory, Cold Spring Harbor, N.Y., United States of America.
Smith (1998) The Quest For Pure DNA: Genomic DNA: The Root Of All Molecular Genetic Research. The Scientist 12(14):21-24.
Smith et al. (1998) A rapid, sensitive, multiplexed assay for detection of viral nucleic acids using the FlowMetrix system. Clin Chem 44(9):2054-2056.
Sohn et al. (2000) Resected adenocarcinoma of the pancreas-616 patients: results, outcomes, and prognostic indicators. J Gastrointest Surg 4:567-579.
Southern (1975) Detection of specific sequences among DNA fragments separated by gel electrophoresis. J Mol Biol 98:503-517.
Strain & Chmielewski (2001) ROCK: a spreadsheet-based program for the generation and analysis of random oligonucleotide primers used in PCR. Bio Techniques 30(6):1286-1291.
Tanaka et al. (1994) Analysis by RNA-PCR of latency and reactivation of herpes simplex virus in multiple neuronal tissues. J Gen Viral 75:2691-2698.
Telenius et al. (1992) Degenerate oligonucleotide-primed PCR: general amplification of target DNA by a single degenerate primer. Genomics 13:718-725.
Theriault et al. (1999) in Schena, ed., DNA Microarrays: A Practical Approach, pp. 101-120, Oxford University Press Inc., New York, N.Y., United States of America.
Tijssen (ed.) (1993) Laboratory Techniques in Biochemistry and Molecular Biology: Hybridization With Nucleic Acid Probes, Part I Theory and Nucleic Acid Preparation, Elsevier Press, New York, N.Y., United States of America.
Tonini et al. (2007) Molecular prognostic factors in patients with pancreatic cancer. Expert Opin Ther Targets 11:1553-1569.
Trinh et al. (2008) The nuclear I kappaB protein I kappaB zeta specifically binds NF-kappaB p50 homodimers and forms a ternary complex on kappaB DNA. J Mol Biol 379:122-135.
Troester et al. (2009) Activation of host wound responses in breast cancer microenvironment. Clin Cancer Res 15:7020-7028.
Troyanskaya et al. (2001) Missing value estimation methods for DNA microarrays. Bioinformatics 17:520-525.
Tusher et al. (2001) Significance analysis of microarrays applied to the ionizing radiation response. Proc Natl Acad Sci USA 98:5116-5121.
U.S. Pat. Nos. 4,729,947; 5,143,854; 5,207,880; 5,230,781; 5,346,603; 5,360,523; 5,534,125; 5,571,388; 5,743,960; 5,800,992; 5,837,832; 5,843,767; 5,846,717; 5,871,918; 5,916,524; 5,965,352; 5,968,745; 5,974,164; 5,985,557; 5,994,069; 6,001,567; 6,017,696; 6,066,457; 6,086,737; 6,090,543; 6,123,819; 6,127,127; 6,162,603; 6,185,561; 6,225,059; 6,229,911; 6,245,508.
Van Cutsem et al. (2009) Phase Ill trial of bevacizumab in combination with gemcitabine and erlotinib in patients with metastatic pancreatic cancer. J Clin Oncol 27:2231-2237.
Van Kerckhoven et al. (1994) Quantification of human immunodeficiency virus in plasma by RNA PCR, viral culture, and p24 antigen detection. J Clin Microbiol 32:1669-1673.
Vignali (2000) Multiplexed particle-based flow cytometric assays. J Immunol Methods 243(1-2):243-255.
Wang et al. (1989) Quantitation of mRNA by the polymerase chain reaction. Proc Natl Acad Sci USA 86:9717-9721.
Warrington et al. (2000) in Schena, ed., Microarray Biochip Technology, pp. 119-148, Eaton Publishing, Natick, Mass., United States of America.
Williams (1989) Optimization strategies for the polymerase chain reaction. Bio Techniques 7:762-769.
Williams et al. (1990) DNA polymorphisms amplified by arbitrary primers are useful as genetic markers. Nucl Acids Res 18(22):6531-6535.
Winter et al. (2006) 1423 pancreaticoduodenectomies for pancreatic cancer: A single-institution experience. J Gastrointest Surg 10:1199-1210; discussion 1210-1211.
Worley et al. (2000) in Schena, ed., Microarray Biochip Technology, pp. 65-86, Eaton Publishing, Natick, Mass., United States of America.
Yang et al. (1998) Hierarchically ordered oxides Science 282:2244-2246.
Yang et al. (2002) Normalization for cDNA microarray data: a robust composite method addressing single and multiple slide systematic variation. Nucleic Acids Res 30:e15.
Yeh et al. (2009) KRAS/BRAF mutation status and ERK1/2 activation as biomarkers for MEK1/2 inhibitor therapy in colorectal cancer. Mol Cancer Ther 8:834-843.
Yeo et al. (1997) Six hundred fifty consecutive pancreaticoduodenectomies in the 1990s: pathology, complications, and outcomes. Ann Surg 226:248-257; discussion 257-260.
Yermilov et al. (2009) Readmissions following pancreaticoduodenectomy for pancreas cancer: a population-based appraisal. Ann Surg Oncol 16:554-561.
Yershov et al. (1996) DNA analysis and diagnostics on oligonucleotide microchips. Proc Natl Acad Sci USA 93:4913-4918.
Zhu et al. (2001) Global analysis of protein activities using proteome chips. Science 293:2101-2105.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for generating a prognostic signature for a subject with pancreatic ductal adenocarcinoma (PDAC), the method comprising determining expression levels for one or more genes selected from the group consisting of Fos B, KLF6, NFKBIZ, ATP4A, GSG1, and SIGLEC11 in PDAC cells obtained from the subject, wherein the determining provides a prognostic signature for the subject.

2. The method of claim 1, comprising determining expression levels for at least four, five, or all six of the genes in PDAC cells obtained from the subject.

3. The method of claim 1, comprising determining expression levels for each of Fos B, KLF6, NFKBIZ, ATP4A, GSG1, and SIGLEC11 in PDAC cells obtained from the subject.

4. The method of claim 1, further comprising comparing the prognostic signature determined to a standard.

5. The method of claim 4, wherein the standard comprises a gene expression profile of the one or more genes obtained from primary PDAC cells obtained from a plurality of subjects with primary PDAC, an expression profile of the one or more genes obtained from metastatic PDAC cells obtained from a plurality of subjects with metastatic PDAC, or both.

6. The method of claim 4, wherein the comparing comprises employing a Single Sample Predictor (SSP).

7. The method of claim 6, wherein the gene expression profile of the one or more genes obtained from primary PDAC cells in the standard comprises a mean expression level for the one or more genes in the primary PDAC cells, the expression profile of the one or more genes obtained from metastatic PDAC cells, or both, and further wherein if the standard comprises both gene expression profiles, the mean expression levels are determined separately for the one or more genes in the primary PDAC cells and the one or more genes in the metastatic PDAC cells.

8. The method of claim 7, wherein the standard comprises both gene expression profiles and the method further comprises assigning with the SSP the prognostic signature to either the mean expression level for the one or more genes in the primary PDAC cells or the mean expression level for the one or more genes in the metastatic PDAC cells.

9. The method of claim 8, wherein the assigning comprises employing a Spearman correlation.

10. The method of claim 9, wherein the assigning step, the comparing step, or both are performed on a suitably-programmed computer.

11. The method of claim 1, wherein the subject is a human.

12. A method for assessing risk of an adverse outcome of a subject with pancreatic ductal adenocarcinoma (PDAC), the method comprising:
(a) determining a mean expression level for one or more genes selected from the group consisting of Fos B, KLF6, NFKBIZ, ATP4A, GSG1, and SIGLEC11 in a biological sample comprising PDAC cells obtained from subject; and
(b) comparing the expression levels determined to .a standard.

13. The method of claim 12, wherein the subject is a human.

14. The method of claim 12, wherein evidence of the expression level is obtained by a method comprising gene expression profiling.

15. The method of claim 14, wherein the gene expression profiling method is a PCR-based method, a microarray based method, or an antibody-based method.

16. The method of claim 14, wherein the expression levels are normalized relative to the expression levels of one or more reference genes, optionally by employing Lowess normalization.

17. The method of claim 12, comprising determining the expression levels of at least four of Fos B, KLF6, NFKBIZ, ATP4A, GSG1, and SIGLEC11.

18. The method of claim 17, comprising determining the expression levels of at least five of Fos B, KLF6, NFKBIZ, ATP4A, GSG1, and SIGLEC11.

19. The method of claim 18, comprising determining the expression levels of all of Fos B, KLF6, NFKBIZ, ATP4A, GSG1, and SIGLEC11.

20. A method for predicting a clinical outcome of a treatment in a subject diagnosed with pancreatic ductal adenocarcinoma (PDAC), the method comprising:
(a) determining the expression level of one or more genes selected from the group consisting of Fos B, KLF6, NFKBIZ, ATP4A, GSG1, and SIGLEC11 in a biological sample comprising PDAC cells obtained from the PDAC of the subject; and
(b) comparing the expression levels determined to a standard, wherein the comparing is predictive of the clinical outcome of the treatment in the subject.

21. The method of claim 20, wherein the clinical outcome is expressed in terms of Recurrence-Free Interval (RFI), Overall Survival (OS), Disease-Free Survival (DFS), or Distant Recurrence-Free Interval (DRFI).

22. The method of claim 20, comprising determining the expression levels of at least four of the one or more genes.

23. The method of claim 22, comprising determining the expression levels of at least five of the one or more genes.

24. The method of claim 23, comprising determining the expression levels of each of the one or more genes.

25. The method of claim 20, where the treatment is selected from among surgical resection of the PDAC, chemotherapy, molecular targeted therapy, immunotherapy, and combinations thereof.

26. The method of claim 20, wherein the standard comprises a gene expression profile of the one or more genes obtained from primary PDAC cells obtained from a plurality of subjects with primary PDAC, an expression profile of the one or more genes obtained from metastatic PDAC cells obtained from a plurality of subjects with metastatic PDAC, or both.

27. The method of claim 20, wherein the comparing comprises employing a Single Sample Predictor (SSP).

28. The method of claim 27, wherein the gene expression profile of the one or more genes obtained from primary PDAC cells in the standard comprises a mean expression level for the one or more genes in the primary PDAC cells, the expression profile of the one or more genes obtained from metastatic PDAC cells, or both, and further wherein if the standard comprises both gene expression profiles, the mean expression levels are determined separately for the one or more genes in the primary PDAC cells and the one or more genes in the metastatic PDAC cells.

29. The method of claim 28, wherein the standard comprises both gene expression profiles, and the method further comprises assigning with the SSP the prognostic signature to either the mean expression level for the one or more genes in the primary PDAC cells or the mean expression level for the one or more genes in the metastatic PDAC cells.

30. The method of claim 29, wherein the assigning comprises employing a Spearman correlation.

31. The method of claim 30, wherein the assigning step, the comparing step, or both are performed on a suitably-programmed computer.

32. The method of claim 20, wherein the subject is a human.

33. A method for predicting a positive or a negative clinical response of a subject with pancreatic ductal adenocarcinoma (PDAC) to a treatment, the method comprising:
(a) determining the expression levels of at least five genes selected from the group consisting of Fos B, KLF6, NFKBIZ, ATP4A, GSG1, and SIGLEC11 in a biological sample comprising PDAC cells obtained from the PDAC of the subject;
(b) comparing the expression levels determined to a first expression profile and a second expression profile, wherein:
(i) the first expression profile is generated by determining the expression levels of the same at least five genes in PDAC cells obtained from a plurality of subjects with primary PDAC;
(ii) the second expression profile is generated by determining the expression levels of the same at least five genes in PDAC cells obtained from a plurality of subjects with metastatic PDAC; and
(iii) assigning the expression levels determined for the at least five genes in the biological sample obtained from the subject to either the first expression profile or the second expression profile,
and further wherein assigning the expression levels determined for the at least five genes in the biological sample obtained from the subject to the first expression profile is indicative of a positive clinical response and assigning the expression levels determined for the at least five genes in the biological sample obtained from the subject to the second expression profile is indicative of a negative clinical response.

34. The method of claim 33, wherein the subject is a human.

35. The method of claim 33, wherein the expression levels of at least five genes determined are normalized as are the expression levels that make up the first and second expression profiles.

36. The method of claim 33, wherein at least one of the first and second expression profiles were generated with Distance Weighted Discrimination (DWD).

37. The method of claim 36, wherein one or more of the determining step, the comparing step, and the assigning step are performed on a suitably-programmed computer.

38. The method of claim 33, wherein the treatment comprises administering gemcitabine to the subject.

39. An array comprising polynucleotides hybridizing to at least five genes selected from among Fos B, KLF6, NFKBIZ, ATP4A, GSG1, and SIGLEC11 or comprising specific peptide or polypeptide gene products of at least five of Fos B, KLF6, NFKBIZ, ATP4A, GSG1, and SIGLEC11.

40. The array of claim 39, wherein each specific peptide or polypeptide gene product present on the array is present thereon in an amount relative to each other specific peptide or polypeptide gene product that is present on the array that is reflective of the expression level of its corresponding gene in pancreatic ductal adenocarcinoma (PDAC) cells obtained from a subject with PDAC.

41. The array of claim 39, wherein the specific peptide or polypeptide gene products are present on the array such that the array is interrogatable with at least one antibody that specifically binds to one of the specific peptide or polypeptide gene products.

42. The array of claim 41, wherein the array comprises at least one specific peptide or polypeptide gene product for each of Fos B, KLF6, NFKBIZ, ATP4A, GSG1, and SIGLEC11.
